(19) United States
(12) Patent Application Publication
(10) Pub. No.: US 2024/0060110 A1
(43) Pub. Date: Feb. 22, 2024

(54) BACILLUS CELL WITH REDUCED LIPASE AND/OR ESTERASE SIDE ACTIVITIES

(71) Applicant: BASF SE, Ludwigshafen (DE)

(72) Inventors: Stefan Jenewein, Ludwigshafen (DE); Max Fabian Felle, Ludwigshafen (DE); Christopher Sauer, Ludwigshafen (DE); Paul Igor Costea, Ludwigshafen (DE)

(21) Appl. No.: 18/030,700

(22) PCT Filed: Oct. 6, 2021
(86) PCT No.: PCT/EP2021/077561

§ 371 Date: Apr. 6, 2023

(30) Foreign Application Priority Data
Jul. 7, 2021 (EP) 21184275.2
Oct. 7, 2021 (EP) 20200558.3

(51) Int. Cl. C12P 21/02; C12N 9/20; C12N 9/16

(52) U.S. Cl. CPC C12P 21/02; C12Y 301/01001; C12N 9/20; C12Y 301/02002; C12N 9/16

(57) ABSTRACT

The present invention provides a modified Bacillus cell for the production of a compound of interest, wherein the Bacillus cell comprises a genetic modification that leads to decreased production of enzymes with lipase and/or esterase activity. The use of compounds of interest produced with said improved Bacillus host cells reduces the formation of malodour and/or off-flavour when used for applications such as washing or cleaning or as feed or food additives.

### FIELD OF THE INVENTION

The present invention provides a modified Bacillus cell for the production of a compound of interest, wherein the Bacillus cell comprises a genetic modification that lead to decreased production of enzymes with lipase and/or esterase activity. The use of compounds of interest produced with said improved Bacillus host cells reduces the formation of malodour and/or off-flavour when used for applications such as washing or cleaning or as feed or food additives.

### BACKGROUND OF THE INVENTION

Microorganisms of the Bacillus genus are widely applied as industrial workhorses for the production of valuable compounds, such as chemicals, polymers and proteins, in particular proteins like washing- and/or cleaning-active enzymes or enzymes used for feed and food applications. The biotechnological production of these useful substances is conducted via fermentation of such Bacillus species and subsequent purification of the product.

Often for the final use of these fermentation-derived substances, in particular enzymes, laborious removal of side products from the fermentation process is necessary. Depending on the processes complete removal of side products is difficult and economically not feasible. These side product of the fermentation process can be toxic and are often the cause for malodour formation or off-flavour (i.e., bad flavour) formation in the product formulation and its application. For some applications complete removal of side products is necessary, for instance, due to regulatory constraints.

WO2006000343 describes the construction of genetically modified Bacillus cells with decreased capability of production of malodorous or toxic compounds during fermentation of such Bacillus cells by targeting genes for inactivation that encode enzymes belonging to five metabolic pathways for the formation of odorous substances like isolvalerian acid, 2-methylbutyric acid, propyl acid, cadaverine and/or putrescine.

Also, lipases can be the cause of odour and flavour formation, a property that is used for the generation of an intense odour in cheese products (cf., e.g., U.S. Pat. No. 4,595,594). However, if these intense flavours or odours are not desired the addition of lipase might be prohibitive as well as in most alternative applications, like washing or cleaning, these intense odours are unwanted and need to be avoided.

WO9807817 describes an approach to reduce lipase-induced malodour in detergents by the addition of an antibody against a specific lipase to detergent compositions.

However, the addition of antibodies against lipases to the final product is very cost-intensive and not very effective. The interaction between antibody and lipase needs specific conditions especially with respect to salt concentration and pH, which are often not fulfilled in the applications to which the antibodies are added. Moreover, the generation of antibodies is laborious and specific to the epitope of the individual lipase used for antibody generation. Furthermore, the relevant lipase side activities produced by Bacillus cells are largely unknown. Various different lipases can be present as side products of Bacillus fermentation, which contribute to malodour and/or off-flavour formation. Not all of these lipases are captured by the specific antibody used. Hence, there is still a need for an improved way of reducing unwanted lipase side activities in product preparations obtained by fermentation of Bacillus cells.

US20030158070 mentions the YitV protein of Bacillus subtilis and classifies YitV as putative serine protease. However, the present inventors revealed that YitV comprises lipase/esterase activity.

### BRIEF SUMMARY OF THE INVENTION

The present inventors identified the YitV and/or YpmR protein to be the major contributors to Bacillus derived lipase side activities and provided a recombinant Bacillus cell comprising a non-native reduction of the YitV and/or YpmR protein expression as a solution for reducing malodour and off-flavour formation in Bacillus derived fermentation products.

Thus, the present invention is directed to a recombinant Bacillus cell comprising a non-native reduction of the YitV and/or YpmR protein expression.

The present invention is also directed to a method for producing a compound of interest, preferably a protein of interest, comprising the fermentation of a Bacillus cell according to the present invention capable of producing the compound of interest, optionally comprising the step of purifying the compound of interest from the fermentation broth.

Moreover, the present invention is directed to a composition comprising the purified or partially purified compound of interest, preferably a protein of interest, obtained by the methods of the present invention and thereby comprising a reduced amount of lipase and/or esterase activity.

Furthermore, the present invention is directed to a method for reducing the formation of malodour or off-flavour in a composition comprising a compound of interest obtained by fermentation of a Bacillus cell comprising the fermentation of a Bacillus cell according to present invention capable of producing a compound of interest, purifying the compound of interest from the fermentation broth and providing a composition comprising the purified compound of interest.

### DETAILED DESCRIPTION OF THE INVENTION

The present invention may be understood more readily by reference to the following detailed description of the embodiments of the invention and the examples included herein.

### Definitions

Unless otherwise noted, the terms used herein are to be understood according to conventional usage by those of ordinary skill in the relevant art.

Before describing in detail exemplary embodiments of the present invention, definitions important for understanding the present invention are given. Unless stated otherwise or apparent from the nature of the definition, the definitions apply to all methods and uses described herein.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

Throughout this application, various publications are referenced. The disclosures of all of these publications and those references cited within those publications in their entireties are hereby incorporated by reference into this application in order to more fully describe the state of the art to which this invention pertains.

It is to be understood that the term “comprising” is not limiting. For the purposes of the present invention the term “consisting of” is considered to be a preferred embodiment of the term “comprising”. If hereinafter a group is defined to comprise at least a certain number of embodiments, this is meant to also encompass a group which preferably consists of these embodiments only.

“Parent” sequence (e.g., “parent enzyme” or “parent protein”) is the starting sequences for introduction of changes (e.g. by introducing one or more amino acid substitutions) of the sequence resulting in “variants” of the parent sequences. Thus, the term “enzyme variant” or “sequence variant” or “protein variant” are used in reference to parent enzymes that are the origin for the respective variant enzymes. Therefore, parent enzymes include wild type enzymes and variants of wild-type enzymes which are used for development of further variants. Variant enzymes differ from parent enzymes in their amino acid sequence to a certain extent; however, variants at least maintain the enzyme properties of the respective parent enzyme. In one embodiment, enzyme properties are improved in variant enzymes when compared to the respective parent enzyme. In one embodiment, variant enzymes have at least the same enzymatic activity when compared to the respective parent enzyme or variant enzymes have increased enzymatic activity when compared to the respective parent enzyme.

In describing the protein variants, the abbreviations for single amino acids used according to the accepted IUPAC single letter or three letter amino acid abbreviation is used.

“Substitutions” are described by providing the original amino acid followed by the number of the position within the amino acid sequence, followed by the substituted amino acid. For example, the substitution of histidine at position 120 with alanine is designated as “His120Ala” or “H120A”.

“Deletions” are described by providing the original amino acid followed by the number of the position within the amino acid sequence, followed by *. Accordingly, the deletion of glycine at position 150 is designated as “Gly150*” or G150*”. Alternatively, deletions are indicated by e.g. “deletion of D183 and G184”.

“Insertions” are described by providing the original amino acid followed by the number of the position within the amino acid sequence, followed by the original amino acid and the additional amino acid. For example, an insertion at position 180 of lysine next to glycine is designated as “Gly180GlyLys” or “G180GK”. When more than one amino acid residue is inserted, such as e.g. a Lys and Ala after Gly180 this may be indicated as: Gly180GlyLysAla or G195GKA.

In cases where a substitution and an insertion occur at the same position, this may be indicated as S99SD+S99A or in short S99AD. In cases where an amino acid residue identical to the existing amino acid residue is inserted, it is clear that degeneracy in the nomenclature arises. If for example a glycine is inserted after the glycine in the above example this would be indicated by G180GG. Variants comprising multiple alterations are separated by “+”, e.g., “Arg170Tyr+Gly195Glu” or “R170Y+G195E” representing a substitution of arginine and glycine at positions 170 and 195 with tyrosine and glutamic acid, respectively. Alternatively, multiple alterations may be separated by space or a comma e.g. R170Y G195E or R170Y, G195E respectively. Where different alterations can be introduced at a position, the different alterations are separated by a comma, e.g. “Arg170Tyr, Glu” and R170T, E, respectively, represents a substitution of arginine at position 170 with tyrosine or glutamic acid. Alternatively, different alterations or optional substitutions may be indicated in brackets, e.g., Arg170[Tyr, Gly] or Arg170{Tyr, Gly} or in short R170 [Y, G] or R170 {Y, G}.

Variants of the parent enzyme molecules may have an amino acid sequence which is at least n percent identical to the amino acid sequence of the respective parent enzyme having enzymatic activity with n being an integer between 50 and 100, preferably 50, 55, 60, 65, 70, 75, 80, 85, 90, 91, 92, 93, 94, 95, 96, 97, 98 or 99 compared to the full length polypeptide sequence. Variant enzymes described herein which are n percent identical when compared to a parent enzyme, have enzymatic activity.

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

After aligning the two sequences, in a second step, an identity value shall be determined from the alignment. Therefore, according to the present invention the following calculation of percent-identity applies:

%−identity=(identical residues/length of the alignment region which is showing the respective sequence of this invention over its complete length)*100. Thus sequence identity in relation to comparison of two amino acid sequences according to this embodiment is calculated by dividing the number of identical residues by the length of the alignment region which is showing the respective sequence of this invention over its complete length. This value is multiplied with 100 to give “%−identity”.

For calculating the percent identity of two DNA sequences the same applies as for the calculation of percent identity of two amino acid sequences with some specifications. For DNA sequences encoding for a protein the pairwise alignment shall be made over the complete length of the coding region from start to stop codon excluding introns. For non-protein-coding DNA sequences the pairwise alignment shall be made over the complete length of the sequence of this invention, so the complete sequence of this invention is compared to another sequence, or regions out of another sequence. Moreover, the preferred alignment program implementing the Needleman and Wunsch algorithm (J. Mol. Biol. (1979) 48, p. 443-453) is “NEEDLE” (The European Molecular Biology Open Software Suite (EMBOSS)) with the programs default parameters (gapopen=10.0, gapextend=0.5 and matrix=EDNAFULL).

Enzyme variants may be defined by their sequence similarity when compared to a parent enzyme. Sequence similarity usually is provided as “% sequence similarity” or “%-similarity”. For calculating sequence similarity in a first step a sequence alignment has to be generated as described above. In a second step, the percent-similarity has to be calculated, whereas percent sequence similarity takes into account that defined sets of amino acids share similar properties, e.g., by their size, by their hydrophobicity, by their charge, or by other characteristics. Herein, the exchange of one amino acid with a similar amino acid is referred to as “conservative mutation”. Enzyme variants comprising conservative mutations appear to have a minimal effect on protein folding resulting in certain enzyme properties being substantially maintained when compared to the enzyme properties of the parent enzyme.

For determination of %-similarity according to this invention the following applies, which is also in accordance with the BLOSUM62 matrix, which is one of the most used amino acids similarity matrix for database searching and sequence alignments
Amino acid A is similar to amino acids S
Amino acid D is similar to amino acids E; N
Amino acid E is similar to amino acids D; K; Q
Amino acid F is similar to amino acids W; Y
Amino acid H is similar to amino acids N; Y
Amino acid I is similar to amino acids L; M; V
Amino acid K is similar to amino acids E; Q; R
Amino acid L is similar to amino acids I; M; V
Amino acid M is similar to amino acids I; L; V
Amino acid N is similar to amino acids D; H; S
Amino acid Q is similar to amino acids E; K; R
Amino acid R is similar to amino acids K; Q
Amino acid S is similar to amino acids A; N; T
Amino acid T is similar to amino acids S
Amino acid V is similar to amino acids I; L; M
Amino acid W is similar to amino acids F; Y
Amino acid Y is similar to amino acids F; H; W.

Conservative amino acid substitutions may occur over the full length of the sequence of a polypeptide sequence of a functional protein such as an enzyme. In one embodiment, such mutations are not pertaining the functional domains of an enzyme. In another embodiment conservative mutations are not pertaining the catalytic centers of an enzyme.

Therefore, according to the present invention the following calculation of percent-similarity applies:

%-similarity=[(identical residues+similar residues)/length of the alignment region which is showing the respective sequence of this invention over its complete length]*100. Thus sequence similarity in relation to comparison of two amino acid sequences herein is calculated by dividing the number of identical residues plus the number of similar residues by the length of the alignment region which is showing the respective sequence of this invention over its complete length. This value is multiplied with 100 to give “%-similarity”.

Especially, variant enzymes comprising conservative mutations which are at least m percent similar to the respective parent sequences with m being an integer between 50 and 100, preferably 50, 55, 60, 65, 70, 75, 80, 85, 90, 91, 92, 93, 94, 95, 96, 97, 98 or 99 compared to the full length polypeptide sequence, are expected to have essentially unchanged enzyme properties. Variant enzymes described herein with m percent-similarity when compared to a parent enzyme, have enzymatic activity.

The term “hybridisation” as defined herein is a process wherein substantially complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, sepharose beads or any other resin. The hybridisation process can furthermore occur with one of the complementary nucleic acids immobilised to a solid support such as a nitro-cellulose or nylon membrane or immobilised by e.g. photolithography to a carrier, including, but not limited to a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

This formation or melting of hybrids is dependent on various parameters, including but not limited thereto the temperature. An increase in temperature favours melting, while a decrease in temperature favours hybridisation. However, this hybrid forming process is not following an applied change in temperature in a linear fashion: the hybridisation process is dynamic, and already formed nucleotide pairs are supporting the pairing of adjacent nucleotides as well. So, with good approximation, hybridisation is a yes-or-no process, and there is a temperature, which basically defines the border between hybridisation and no hybridisation. This temperature is the melting temperature (Tm). Tm is the temperature in degrees Celsius, at which 50% of all molecules of a given nucleotide sequence are hybridised into a double strand, and 50% are present as single strands.

The melting temperature (Tm) is dependent from the physical properties of the analysed nucleic acid sequence and hence can indicate the relationship between two distinct sequences. However, the melting temperature (Tm) is also influenced by various other parameters, which are not directly related with the sequences, and the applied conditions of the hybridization experiment must be taken into account. For example, an increase of salts (e.g. monovalent cations) is resulting in a higher Tm.

Tm for a given hybridisation condition can be determined by doing a physical hybridisation experiment, but Tm can also be estimated in silico for a given pair of DNA sequences. In this embodiment, the equation of Meinkoth and Wahl (Anal. Biochem., 138:267-284, 1984) is used for stretches having a length of 50 or more bases: Tm=81.5° C.+16.6 (log M)+0.41 (% GC)−0.61 (% form)−500/L.

M is the molarity of monovalent cations, % GC is the percentage of guanosine and cytosine nucleotides in the DNA stretch, % form is the percentage of formamide in the hybridisation solution, and L is the length of the hybrid in base pairs. The equation is for salt ranges of 0.01 to 0.4 M and % GC in ranges of 30% to 75%.

While above Tm is the temperature for a perfectly matched probe, Tm is reduced by about 1° C. for each 1% of mismatching (Bonner et al., J. Mol. Biol. 81: 123-135, 1973): Tm=[81.5° C.+16.6(log M)+0.41 (% GC)−0.61 (% formamide)−500/L]−% non-identity.

This equation is useful for probes having 35 or more nucleotides and is widely referenced in scientific method literature (e.g. in: “Recombinant DNA Principles and Methodologies”, James Greene, Chapter “Biochemistry of Nucleic acids”, Paul S. Miller, page 55; 1998, CRC Press), in many patent applications (e.g. in: U.S. Pat. No. 7,026,149), and also in data sheets of commercial companies (e.g. “Equations for Calculating Tm” from www.genomics.agilent.com).

Other formulas for Tm calculations, which are less preferred in this embodiment, might be only used for the indicated cases: For DNA-RNA hybrids (Casey, J. and Davidson, N. (1977) Nucleic Acids Res., 4:1539): Tm=79.8° C.+18.5 (log M)+0.58 (% GC)+11.8 (% GC*% GC)−0.5 (% form)−820/L.

For RNA-RNA hybrids (Bodkin, D. K. and Knudson, D. L. (1985) J. Virol. Methods, 10: 45): Tm=79.8° C.+18.5 (log M)+0.58 (% GC)+11.8 (% GC*% GC)−0.35 (% form)−820/L.

For oligonucleotide probes of less than 20 bases (Wallace, R. B., et al. (1979) Nucleic Acid Res. 6: 3535): Tm=2×n(A+T)+4×n(G+C), with n being the number of respective bases in the probe forming a hybrid.

For oligonucleotide probes of 20-35 nucleotides, a modified Wallace calculation could be applied: Tm=22+1.46 n(A+T)+2.92 n(G+C), with n being the number of respective bases in the probe forming a hybrid.

For other oligonucleotides, the nearest-neighbour model for melting temperature calculation should be used, together with appropriate thermodynamic data:

Tm=(Z(AHd)+AHi)/((ASd)+ASi+ASself+R×In(cT/b))+16.6 log[Na+]−273.15 (Breslauer, K. J., Frank, R., Blöcker, H., Marky, L. A. 1986 Predicting DNA duplex stability from the base sequence. Proc. Natl Acad. Sci. USA 833746-3750; Alejandro Panjkovich, Francisco Melo, 2005. Comparison of different melting temperature calculation methods for short DNA sequences. Bioinformatics, 21 (6): 711-722)
where:
Tm is the melting temperature in degrees Celsius;
ΣZ(ΔHd) and Σ(ΔSd) are sums of enthalpy and entropy (correspondingly), calculated over all internal nearest-neighbor doublets;
ΔSself is the entropic penalty for self-complementary sequences;
ΔHi and ΔSi are the sums of initiation enthalpies and entropies, respectively;
R is the gas constant (fixed at 1.987 cal/K-mol);
cT is the total strand concentration in molar units;
constant b adopts the value of 4 for non-self-complementary sequences or equal to 1 for duplexes of self-complementary strands or for duplexes when one of the strands is in significant excess.

The thermodynamic calculations assume that the annealing occurs in a buffered solution at pH near 7.0 and that a two-state transition occurs.

Thermodynamic values for the calculation can be obtained from Table 1 in (Alejandro Panjkovich, Francisco Melo, 2005. Comparison of different melting temperature calculation methods for short DNA sequences. Bioinformatics, 21 (6): 711-722), or from the original research papers (Breslauer, K. J., Frank, R., B16cker, H., Marky, L. A. 1986 Predicting DNA duplex stability from the base sequence. Proc. Natl Acad. Sci. USA 833746-3750; SantaLucia, J., Jr, Allawi, H. T., Seneviratne, P. A. 1996 Improved nearest-neighbor parameters for predicting DNA duplex stability. Biochemistry 353555-3562; Sugimoto, N., Nakano, S., Yoneyama, M., Honda, K. 1996 Improved thermodynamic parameters and helix initiation factor to predict stability of DNA duplexes. Nucleic Acids Res. 244501-4505).

For an in silico estimation of Tm according to this embodiment, first a set of bioinformatic sequence alignments between the two sequences are generated. Such alignments can be generated by various tools known to a person skilled in the art, like programs “Blast” (NCBI), “Water” (EMBOSS) or “Matcher” (EMBOSS), which are producing local alignments, or “Needle” (EMBOSS), which is producing global alignments. Those tools should be applied with their default parameter setting, but also with some parameter variations. For example, program “MATCHER” can be applied with various parameter for gapopen/gapextend (like 14/4; 14/2; 14/5; 14/8; 14/10; 20/2; 20/5; 20/8; 20/10; 30/2; 30/5; 30/8; 30/10; 40/2; 40/5; 40/8; 40/10; 10/2; 10/5; 10/8; 10/10; 8/2; 8/5; 8/8; 8/10; 6/2; 6/5; 6/8; 6/10) and program “WATER” can be applied with various parameter for gapopen/gapextend (like 10/0.5; 10/1; 10/2; 10/3; 10/4; 10/6; 15/1; 15/2; 15/3; 15/4; 15/6; 20/1; 20/2; 20/3; 20/4; 20/6; 30/1; 30/2; 30/3; 30/4; 30/6; 45/1; 45/2; 45/3; 45/4; 45/6; 60/1; 60/2; 60/3; 60/4; 60/6), and also these programs shall be applied by using both nucleotide sequences as given, but also with one of the sequences in its reverse complement form. For example, BlastN (NCBI) can be applied with an increased e-value cut-off (e.g. e+1 or even e+10) to also identify very short alignments, especially in data bases of small sizes.

Important is that local alignments are considered, since hybridisation may not necessarily occur over the complete length of the two sequences, but may be best at distinct regions, which then are determining the actual melting temperature. Therefore, from all created alignments, the alignment length, the alignment % GC content (in a more accurate manner, the % GC content of the bases which are matching within the alignment), and the alignment identity has to be determined. Then the predicted melting temperature (Tm) for each alignment has to be calculated. The highest calculated Tm is used to predict the actual melting temperature.

The term “hybridisation over the complete sequence of the invention” as defined herein means that for sequences longer than 300 bases when the sequence of the invention is fragmented into pieces of about 300 to 500 bases length, every fragment must hybridise. For example, a DNA can be fragmented into pieces by using one or a combination of restriction enzymes. A bioinformatic in silico calculation of Tm is then performed by the same procedure as described above, just done for every fragment. The physical hybridisation of individual fragments can be analysed by standard Southern analysis, or comparable methods, which are known to a person skilled in the art.

The term “stringency” as defined herein is describing the ease by which hybrid formation between two nucleotide sequences can take place. Conditions of a “higher stringency” require more bases of one sequence to be paired with the other sequence (the melting temperature Tm is lowered in conditions of “higher stringency”), conditions of “lower stringency” allow some more bases to be unpaired. Hence the degree of relationship between two sequences can be estimated by the actual stringency conditions at which they are still able to form hybrids. An increase in stringency can be achieved by keeping the experimental hybridisation temperature constant and lowering the salts concentrations, or by keeping the salts constant and increasing the experimental hybridisation temperature, or a combination of these parameter. Also an increase of formamide will increase the stringency. The skilled artisan is aware of additional parameters which may be altered during hybridisation and which will either maintain or change the stringency conditions (Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

A typical hybridisation experiment is done by an initial hybridisation step, which is followed by one to several washing steps. The solutions used for these steps may contain additional components, which are preventing the degradation of the analyzed sequences and/or prevent unspecific background binding of the probe, like EDTA, SDS, fragmented sperm DNA or similar reagents, which are known to a person skilled in the art (Sambrook et al. (2001) Molecular Cloning: a laboratory manual, 3^{rd} Edition, Cold Spring Harbor Laboratory Press, CSH, New York or to Current Protocols in Molecular Biology, John Wiley & Sons, N.Y. (1989 and yearly updates).

A typical probe for a hybridisation experiment is generated by the random-primed-labelling method, which was initially developed by Feinberg and Vogelstein (Anal. Biochem., 132 (1), 6-13 (1983); Anal. Biochem., 137 (1), 266-7 (1984) and is based on the hybridisation of a mixture of all possible hexanucleotides to the DNA to be labelled. The labelled probe product will actually be a collection of fragments of variable length, typically ranging in sizes of 100-1000 nucleotides in length, with the highest fragment concentration typically around 200 to 400 bp. The actual size range of the probe fragments, which are finally used as probes for the hybridisation experiment, can also be influenced by the used labelling method parameter, subsequent purification of the generated probe (e.g. agarose gel), and the size of the used template DNA which is used for labelling (large templates can e.g. be restriction digested using a 4 bp cutter, e.g. Haelll, prior labeling).

For the present invention, the sequence described herein is analysed by a hybridisation experiment, in which the probe is generated from the other sequence, and this probe is generated by a standard random-primed-labelling method. For the present invention, the probe is consisting of a set of labelled oligonucleotides having sizes of about 200-400 nucleotides. A hybridisation between the sequence of this invention and the other sequence means, that hybridisation of the probe occurs over the complete sequence of this invention, as defined above. The hybridisation experiment is done by achieving the highest stringency by the stringency of the final wash step. The final wash step has stringency conditions comparable to the stringency conditions of at least Wash condition 1: 1.06×SSC, 0.1% SDS, 0% formamide at 50° C., in another embodiment of at least Wash condition 2: 1.06×SSC, 0.1% SDS, 0% formamide at 55° C., in another embodiment of at least Wash condition 3: 1.06×SSC, 0.1% SDS, 0% formamide at 60° C., in another embodiment of at least Wash condition 4: 1.06×SSC, 0.1% SDS, 0% formamide at 65° C., in another embodiment of at least Wash condition 5: 0.52×SSC, 0.1% SDS, 0% formamide at 65° C., in another embodiment of at least Wash condition 6: 0.25×SSC, 0.1% SDS, 0% formamide at 65° C., in another embodiment of at least Wash condition 7: 0.12×SSC, 0.1% SDS, 0% formamide at 65° C., in another embodiment of at least Wash condition 8: 0.07×SSC, 0.1% SDS, 0% formamide at 65° C.

A “low stringent wash” has stringency conditions comparable to the stringency conditions of at least Wash condition 1, but not more stringent than Wash condition 3, wherein the wash conditions are as described above.

A “high stringent wash” has stringency conditions comparable to the stringency conditions of at least Wash condition 4, in another embodiment of at least Wash condition 5, in another embodiment of at least Wash condition 6, in another embodiment of at least Wash condition 7, in another embodiment of at least Wash condition 8, wherein the wash conditions are as described above.

The term “heterologous” (or exogenous or foreign or recombinant or non-native) polypeptide is defined herein as a polypeptide that is not native to the host cell, a polypeptide native to the host cell in which structural modifications, e.g., deletions, substitutions, and/or insertions, have been made by recombinant DNA techniques to alter the native polypeptide, or a polypeptide native to the host cell whose expression is quantitatively altered or whose expression is directed from a genomic location different from the native host cell as a result of manipulation of the DNA of the host cell by recombinant DNA techniques, e.g., a stronger promoter. Similarly, the term “heterologous” (or exogenous or foreign or recombinant or non-native) polynucleotide refers to a polynucleotide that is not native to the host cell, a polynucleotide native to the host cell in which structural modifications, e.g., deletions, substitutions, and/or insertions, have been made by recombinant DNA techniques to alter the native polynucleotide, or a polynucleotide native to the host cell whose expression is quantitatively altered as a result of manipulation of the regulatory elements of the polynucleotide by recombinant DNA techniques, e.g., a stronger promoter, or a polynucleotide native to the host cell, but integrated not within its natural genetic environment as a result of genetic manipulation by recombinant DNA techniques.

With respect to two or more polynucleotide sequences or two or more amino acid sequences, the term “heterologous” is used to characterized that the two or more polynucleotide sequences or two or more amino acid sequences are naturally not occurring in the specific combination with each other.

For the purposes of the invention, “recombinant” (or transgenic) with regard to a cell or an organism means that the cell or organism contains a heterologous polynucleotide which is introduced by man by gene technology and with regard to a polynucleotide includes all those constructions brought about by man by gene technology/recombinant DNA techniques in which either
(a) the sequence of the polynucleotide or a part thereof, or
(b) one or more genetic control sequences which are operably linked with the polynucleotide, including but not limited thereto a promoter, or
(c) both a) and b)
are not located in their wildtype genetic environment or have been modified.

The term “native” (or wildtype or endogenous) cell or organism and “native” (or wildtype or endogenous) polynucleotide or polypeptide refers to the cell or organism as found in nature and to the polynucleotide or polypeptide in question as found in a cell in its natural form and genetic environment, respectively (i.e., without there being any human intervention).

As used herein the term “coding region” (or ORF—open reading frame) when used in reference to a structural gene refers to the nucleotide sequences which encode the amino acids found in the nascent polypeptide as a result of translation of a mRNA molecule. The coding region is bounded on the 5-side by the nucleotide triplet “ATG” which encodes the initiator methionine and on the 3′-side by one of the three triplets which specify stop codons (i.e., TAA, TAG, TGA). Alternatively, the nucleotide triplet can be “GTG” or “TTG” and is recognized as the start nucleotide triplet as 5′ to said nucleotide triplet the ribosome binding site (Shine Dalgarno) is located in a distance of 4 nucleotides to 12 nucleotides. Genomic forms of a gene may also include sequences located on both the 5′- and 3′-end of the sequences which are present on the RNA transcript, i.e. these sequences are located 5′ or 3′ to the non-translated sequences present on the mRNA transcript. Furthermore the 5′ and 3′ sequences also referred to as ‘flanking’ sequences or regions may further contain regulatory sequences such as within the 5′ sequence promoters and enhancers which control or influence the transcription of the gene and the ribosome binding site (Shine Dalgarno) which controls or influences translation of the mRNA and within the 3′ sequence contain sequences which direct the termination of transcription and posttranscriptional cleavage.

Herein, “complementary” or “complementarity” refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT-3′ is complementary to the sequence 5′-ACT-3′. Complementarity can be “partial” or “total.” “Partial” complementarity is where one or more nucleic acid bases are not matched according to the base pairing rules. “Total” or “complete” complementarity between nucleic acid molecules is where each and every nucleic acid base is matched with another base under the base pairing rules. The degree of complementarity between nucleic acid molecule strands has significant effects on the efficiency and strength of hybridization between nucleic acid molecule strands. A “complement” of a nucleic acid sequence as used herein refers to a nucleotide sequence whose nucleic acid molecules show total complementarity to the nucleic acid molecules of the nucleic acid sequence.

The term “gene expression” means the transcription of a specific gene or specific genes or specific nucleic acid construct. The term “gene expression” in particular means the transcription of a gene or genes or genetic construct into structural RNA (e.g., rRNA, tRNA) or mRNA with or without subsequent translation of the latter into a protein. The process includes transcription of DNA and processing of the resulting mRNA product. The term “expression of a protein” specifically means the transcription of a gene or genes or genetic construct into mRNA with subsequent translation of the latter into a protein.

The term “reduction of expression of a protein” shall mean a reduced amount of the protein being produced by the cell, for instance, by a reduced transcription or reduced translation.

The term “non-native reduction of expression of a protein” shall mean a reduced amount of the protein being produced by the cell achieved by any kind genetic modification by human intervention, in particular recombinant DNA techniques, applied to the cell compared to the cell not subject to the genetic modification, i.e., the respective parent host cell.

The term “non-native complete or partial deletion of a gene” shall mean any kind genetic modification by human intervention, in particular recombinant DNA techniques, applied to the cell compared to the cell not subject to the genetic modification resulting in a complete for partial removal of the gene, i.e., the respective parent host cell.

The term “inactivation” (also called herein “functional inactivation”) in connection with a gene means that the functionality of said gene has been reduced compared to the functionality of the gene in the parent host cell, so that the gene produces no active protein or a reduced amount of active protein, preferably the inactivated gene produces no active protein. A parent host cell is a host cell, which differs from the host cell of the present invention only in that the gene has not been inactivated, i.e., a corresponding host cell with a functional gene. The functionality of the gene can be inactivated by various means known to the person skilled in the art. In a preferred embodiment, the yitV gene of the parent host cell is inactivated so that the host cell does not produce active YitV protein. In a preferred embodiment, the ypmR gene of the parent host cell is inactivated so that the host cell does not produce active YpmR protein. In a particularly preferred embodiment, the yitV and the ypmR gene of the parent host cell are inactivated so that the host cell does not produce active YitV protein and no active YpmR protein.

The terms “genome” or “genomic DNA” is referring to the heritable genetic information of a host organism. In eukaryotes said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus. In prokaryotes said genomic DNA comprises the chromosomal DNA within the bacterial cell.

As used herein, a “flanking” sequence or region refers to any sequence that is either 5′ also referred to as “upstream’ or 3′ also referred to as “downstream” of the sequence being discussed (e.g. for sequences A-B-C, sequence B is flanked by the A and C sequences)

The terms “polynucleotide”, “nucleic acid sequence”, “nucleotide sequence”, “nucleic acid”, “nucleic acid molecule” are used interchangeably herein and refer to nucleotides, either ribonucleotides or deoxyribonucleotides or a combination of both, in a polymeric unbranched form of any length. “Polynucleotides” are composed of monomers, which are “nucleotides” made of three components: a pentose sugar, a phosphate group, and a nitrogenous base.

The term “selectable marker” (or marker) comprises genes encoding for polypeptides when being expressed in a bacterial host cell under conditions that effectively express said marker polypeptide allow growth or increased growth compared to a control host cell. Such positive selection by, e.g. antibiotic resistance markers such as ampicillin, kanamycin, erythromycin, chloramphenicol or tetracycline and auxotrophic resistance markers have been used to e.g. retain plasmids within a cell, or to select on host cells with successful gene integration.

Likewise, “counterselection markers” are understood as genes encoding for polynucleotides when being expressed in a bacterial host cells under conditions that effectively express said counterselection polypeptide, optionally in the presence of an effective amount of counterselection agent for the counterselection polypeptide (if required) are toxic to the host cell and/or result in decreased growth compared to a control host cell. Counterselection markers are known in the art and have been used for plasmid removal, selection marker removal or removal of integrated plasmid from the host cell chromosome.

The term “foreign” (or exogenous, or non-native) refers to any nucleic acid molecule (e.g., gene sequence) which is introduced into the genome of a cell by experimental manipulations and may include sequences found in that cell so long as the introduced sequence contains some modification (e.g., a point mutation, the presence of a selectable marker gene, etc.) and is therefore distinct relative to the naturally-occurring sequence.

The term “nucleic acid construct” as used herein refers to a nucleic acid molecule, either single- or double-stranded, which is isolated from a naturally occurring gene or is modified to contain segments of nucleic acids in a manner that would not otherwise exist in nature or is synthetic. The term “nucleic acid construct” is synonymous with the term “expression cassette” when the nucleic acid construct contains the control sequences required for expression of a polynucleotide.

The term “control sequence” is defined herein to include all sequences affecting the expression of a polynucleotide, including but not limited thereto, the expression of a polynucleotide encoding a polypeptide. Each control sequence may be native or foreign to the polynucleotide or native or foreign to each other. Such control sequences include, but are not limited to, promoter sequence, 5′-UTR (also called leader sequence), ribosomal binding site (RBS, shine dalgarno sequence), 3′-UTR, and transcription start and stop sites.

The term “functional linkage” or “operably linked” with respect to regulatory elements, is to be understood as meaning the sequential arrangement of a regulatory element (including but not limited thereto a promoter) with a nucleic acid sequence to be expressed and, if appropriate, further regulatory elements (including but not limited thereto a terminator) in such a way that each of the regulatory elements can fulfil its intended function to allow, modify, facilitate or otherwise influence expression of said nucleic acid sequence. For example, a control sequence is placed at an appropriate position relative to the coding sequence of the polynucleotide sequence such that the control sequence directs the expression of the coding sequence of a polypeptide. Preferred arrangements are those in which the nucleic acid sequence to be expressed recombinantly is positioned behind the sequence acting as promoter, so that the two sequences are linked covalently to each other. In one embodiment, the nucleic acid sequence to be transcribed is located behind the promoter in such a way that the transcription start is identical with the desired beginning of the RNA. Functional linkage, and an expression construct, can be generated by means of customary recombination and cloning techniques as described (e.g., Sambrook, J. and Russell, D. W. Molecular cloning. A laboratory manual, 3rd ed, Cold Spring Harbor Laboratory Press, Cold Spring Harbor, N Y. 2001.); Silhavy et al. (1984) Experiments with Gene Fusions, Cold Spring Harbor Laboratory, Cold Spring Harbor (NY); Ausubel et al. (1987) Current Protocols in Molecular Biology, Greene Publishing Assoc. and Wiley Interscience; Gelvin et al. (Eds) (1990) Plant Molecular Biology Manual; Kluwer Academic Publisher, Dordrecht, The Netherlands; Plant Molecular Biology Labfax (1993) by R. D. D. Croy, published by BIOS Scientific Publications Ltd (UK) and Blackwell Scientific Publications (UK)). However, further sequences, including but not limited thereto a sequence, which acts as a linker with specific cleavage sites for restriction enzymes, or as a signal peptide, may also be positioned between the two sequences. The insertion of sequences may also lead to the expression of fusion proteins.

A “promoter” or “promoter sequence” is a nucleotide sequence located upstream of a gene on the same strand as the gene that enables that gene's transcription. Promoter is followed by the transcription start site of the gene. Promoter is recognized by RNA polymerase (together with any required transcription factors), which initiates transcription. A functional fragment or functional variant of a promoter is a nucleotide sequence which is recognizable by RNA polymerase, and capable of initiating transcription.

An “active promoter fragment”, “active promoter variant”, “functional promoter fragment” or “functional promoter variant” describes a fragment or variant of the nucleotide sequences of a promoter, which still has promoter activity.

The term “transcription start site” or “transcriptional start site” shall be understood as the location where the transcription starts at the 5′ end of a gene sequence. In prokaryotes the first nucleotide, referred to as +1 is in general an adenosine (A) or guanosine (G) nucleotide. In this context, the terms “sites” and “signal” can be used interchangeably herein.

When used herein the term “coding sequence” means a nucleotide sequence, which directly specifies the amino acid sequence of its protein product. The boundaries of the coding sequence are generally determined by an open reading frame, which usually begins with the ATG start codon or alternative start codons such as GTG, CTG or TTG and ends with a stop codon such as TAA, TAG, or TGA. The coding sequence may be a DNA, cDNA, synthetic, or recombinant nucleotide sequence. The start codon can also be named herein as “translational start signal” or “translational start site”. The stop codon can also be named herein as “translational stop signal” or “translational stop site”.

The term “expression vector” is defined herein as a linear or circular DNA molecule that comprises a polynucleotide that is operably linked to one or more control sequences that provides for the expression of the polynucleotide.

The term “host cell”, as used herein, includes any cell type that is susceptible to transformation, transfection, transduction, conjugation, and the like with a nucleic acid construct or expression vector.

The term “introduction” and variations thereof are defined herein as the transfer of a DNA into a host cell. The introduction of a DNA into a host cell can be accomplished by any method known in the art, including, the not limited to, transformation, transfection, transduction, conjugation, or DNA uptake mechanisms (e.g. competency, or induced competency) well known for Bacillus (WO08079895). Likewise, the term “incoming” DNA is referred to as the DNA transferred into a bacterial host cell.

The term “donor cell” is defined herein as a cell that is the source of DNA introduced by any means to another cell.

The term “recipient cell” is defined herein as a cell into which DNA is introduced.

The term “fermentation in industrial scale” (also called large-scale fermentation) refers to fermentation processes with fermenter volumes of greater than or equal to 20 liters.

The term “operon” is understood herein as a unit of genomic DNA, containing a single promoter, and one or more genes, all of which are transcribed from that single promoter. The genes in the operon may overlap, or may have untranslated regions (UTRs) between each other. These UTRs may optionally have additional control elements, affecting translational efficiency.

As used herein, the term “vector” refers to an extrachromosomal circular DNA. A vector may be capable of autonomously replicating in the host cell, or not. The term “plasmid” refers to an extrachromosomal circular DNA, i.e. a vector that is autonomously replicating in the host cell. Thus, a plasmid is understood as extrachromosomal vector (and shall not be stably integrated in the bacterial chromosome).

Herein, an “enzyme having esterase activity is a hydrolase enzyme that splits esters into an acid and an alcohol in a chemical reaction with water called hydrolysis.

The term “purification” or “purifying” refers to a process in which at least one component, e.g., a protein of interest, is separated from at least another component, e.g., a particulate matter of a fermentation broth, and transferred into a different compartment or phase, wherein the different compartments or phases do not necessarily need to be separated by a physical barrier. Examples of such different compartments are two compartments separated by a filtration membrane or cloth, i.e., filtrate and retentate; examples of such different phases are pellet and supernatant or cake and filtrate, respectively.

### DETAILED DESCRIPTION

Enzymes Having Lipase and/or Esterase Activity

The present invention is directed to a Bacillus cell comprising a reduced expression of one or more proteins having lipase and/or esterase activity. Preferably, the recombinant Bacillus cell comprising a non-native reduction of the YitV and/or YpmR protein expression. Most preferably, the recombinant Bacillus cell comprising a non-native reduction of the YitV and YpmR protein expression.

Preferably, the YitV protein, which expression is reduced, is selected from the group consisting of:
(a) a YitV protein having at least 70%, at least 75%, at least 80%, at least 85%, at least 90%, at least 92%, at least 95%, at least 96%, at least 97%, at least 98%, at least 99%, or even 100% identity with SEQ ID NO: 2;
(b) a YitV protein encoded by a polynucleotide having at least 70%%, at least 75%, at least 80%, at least 85%, at least 90%, at least 92%, at least 95%, at least 96%, at least 97%, at least 98%, at least 99%, or even 100% identity with SEQ ID NO: 32;
(c) a YitV protein encoded by a polynucleotide that hybridizes under high stringency conditions with (i) a polynucleotide comprising SEQ ID NO: 32 or (ii) the full-length complement of (i);
(d) a variant of the YitV protein of SEQ ID NO: 2 comprising a substitution, deletion, and/or insertion at one or more positions and having lipase and/or esterase activity;
(e) a YitV protein encoded by a polynucleotide that differs from SEQ ID NO: 32 due to the degeneracy of the genetic code.

Preferably, the YitV protein, which expression is reduced, is a YitV protein having at least 80%, at least 85%, at least 90%, at least 92%, at least 95%, at least 96%, at least 97%, at least 98%, at least 99%, or even 100% identity with SEQ ID NO: 2.

Preferably, the YpmR protein, which expression is reduced, is selected from the group consisting of:
(a) a YpmR protein having at least 70%, at least 75%, at least 80%, at least 85%, at least 90%, at least 92%, at least 95%, at least 96%, at least 97%, at least 98%, at least 99%, or even 100% identity with SEQ ID NO: 10;
(b) a YpmR protein encoded by a polynucleotide having at least 70%, at least 75%, at least 80%, at least 85%, at least 90%, at least 92%, at least 95%, at least 96%, at least 97%, at least 98%, at least 99%, or even 100% identity with SEQ ID NO: 40;
(c) a YpmR protein encoded by a polynucleotide that hybridizes under high stringency conditions with (i) a polynucleotide comprising SEQ ID NO: 40 or (ii) the full-length complement of (i);
(d) a variant of the YpmR protein of SEQ ID NO: 10 comprising a substitution, deletion, and/or insertion at one or more positions and having lipase and/or esterase activity; (e) a YpmR protein encoded by a polynucleotide that differs from SEQ ID NO: 40 due to the degeneracy of the genetic code.

Preferably, the YpmR protein, which expression is reduced, is a YpmR protein having at least 80%, at least 85%, at least 90%, at least 92%, at least 95%, at least 96%, at least 97%, at least 98%, at least 99%, or even 100% identity with SEQ ID NO: 10.

Preferably, the recombinant Bacillus cell comprising a non-native reduction of the expression of a protein having lipase and/or esterase activity, wherein the protein comprises an amino acid sequence having at least 70%, at least 75%, at least 80%, at least 85%, at least 90%, at least 92%, at least 95%, at least 96%, at least 97%, at least 98%, at least 99%, or even 100% to SEQ ID NO: 2 or 10.

In one embodiment, the present invention refers to a recombinant Bacillus cell, preferably a Bacillus licheniformis cell, comprising an inactivation of the yitV and ypmR gene.

Preferably the yitV gene encodes a YitV protein that is selected from the group consisting of:
(a) a YitV protein having at least 70% identity with SEQ ID NO: 2;
(b) a YitV protein encoded by a polynucleotide having at least 70% identity with SEQ ID NO: 32;
(c) a YitV protein encoded by a polynucleotide that hybridizes under high stringency conditions with (i) a polynucleotide comprising SEQ ID NO: 32 or (ii) the full-length complement of (i);
(d) a variant of the YitV protein of SEQ ID NO: 2 comprising a substitution, deletion, and/or insertion at one or more positions and having lipase and/or esterase activity;
(e) a YitV protein encoded by a polynucleotide that differs from SEQ ID NO: 32 due to the degeneracy of the genetic code.

Preferably, the ypmR gene encodes a YpmR protein that is selected from the group consisting of:
(a) a YpmR protein having at least 70% identity with SEQ ID NO: 10;
(b) a YpmR protein encoded by a polynucleotide having at least 70% identity with SEQ ID NO: 40;
(c) a YpmR protein encoded by a polynucleotide that hybridizes under high stringency conditions with (i) a polynucleotide comprising SEQ ID NO: 40 or (ii) the full-length complement of (i);
(d) a variant of the YpmR protein of SEQ ID NO: 10 comprising a substitution, deletion, and/or insertion at one or more positions and having lipase and/or esterase activity;
(e) a YpmR protein encoded by a polynucleotide that differs from SEQ ID NO: 40 due to the degeneracy of the genetic code.

However, also other proteins having lipase and/or esterase activity can be reduced in their expression level in the Bacillus cell. Preferably one or more of the following proteins having lipase and/or esterase activity or variants or homologues thereof is reduced in the expression in the Bacillus cell as shown in Table 1:

| Gene | | | | |
|---|---|---|---|---|
| name | Function | PRT | DNA | Accession |
| yhfR | putative phosphatase | SEQ ID 1 | SEQ ID 31 | PF14606.7 |
| yitV | putative peptidase | SEQ ID 2 | SEQ ID 32 | PF02230.17 |
| yesT | rhamnogalacturonan acety- | SEQ ID 3 | SEQ ID 33 | PF13472.7 |
| | lesterase | | | |
| yesT | rhamnogalacturonan acety- | SEQ ID 3 | SEQ ID 33 | PF00657.23 |
| | lesterase | | | |
| unknown | putative carbohydrate esterase | SEQ ID 4 | SEQ ID 34 | PF13472.7 |
| unknown | putative carbohydrate esterase | SEQ ID 4 | SEQ ID 34 | PF00657.23 |
| yesY | putative rhamnogalac- | SEQ ID 5 | SEQ ID 35 | PF13472.7 |
| | turonan acetylesterase | | | |
| yes Y | putative rhamnogalac- | SEQ ID 5 | SEQ ID 35 | PF00657.23 |
| | turonan acetylesterase | | | |
| unknown | hypothetical protein | SEQ ID 6 | SEQ ID 36 | PF01764.26 |
| fliG | flagellar motor switch protein | SEQ ID 7 | SEQ ID 37 | PF03280.15 |
| yisY | AB hydrolase superfamily | SEQ ID 8 | SEQ ID 38 | PF01764.26 |
| | protein | | | |
| yisY | AB hydrolase superfamily | SEQ ID 8 | SEQ ID 38 | PF03583.15 |
| | protein | | | |
| yisY | AB hydrolase superfamily | SEQ ID 8 | SEQ ID 38 | PF02230.17 |
| | protein | | | |
| mhqD | putative hydrolase | SEQ ID 9 | SEQ ID 39 | PF02230.17 |
| ypmR | putative esterase | SEQ ID 10 | SEQ ID 40 | PF13472.7 |
| ypmR | putative esterase | SEQ ID 10 | SEQ ID 40 | PF00657.23 |
| ypmR | putative esterase | SEQ ID 10 | SEQ ID 40 | PF14606.7 |
| lip | putative lipase | SEQ ID 11 | SEQ ID 41 | PF01674.19 |
| yqiL | hypothetical protein | SEQ ID 12 | SEQ ID 42 | PF01764.26 |
| yqeF | putative lipoprotein | SEQ ID 13 | SEQ ID 43 | PF13472.7 |
| yqeF | putative lipoprotein | SEQ ID 13 | SEQ ID 43 | PF00657.23 |
| unknown | putative lipolytic enzyme | SEQ ID 14 | SEQ ID 44 | PF01764.26 |
| unknown | putative lipolytic enzyme | SEQ ID 14 | SEQ ID 44 | PF02230.17 |
| ytaP | putative hydrolase | SEQ ID 15 | SEQ ID 45 | PF03583.15 |
| ytpA | phospholipase | SEQ ID 16 | SEQ ID 46 | PF02230.17 |
| ytmA | putative dipeptidylpeptidase | SEQ ID 17 | SEQ ID 47 | PF02230.17 |
| ytmA | putative dipeptidylpeptidase | SEQ ID 17 | SEQ ID 47 | PF03583.15 |
| ytxM | 2-succinyl-6-hydroxy-2, 4- | SEQ ID 18 | SEQ ID 48 | PF02230.17 |
| | cyclohexadiene-1-carbox- | | | |
| | ylate synthase | | | |
| yugF | alpha/beta hydrolase | SEQ ID 19 | SEQ ID 49 | PF02230.17 |
| yugF | alpha/beta hydrolase | SEQ ID 19 | SEQ ID 49 | PF01764.26 |
| unknown | dipeptidylpeptidase | SEQ ID 20 | SEQ ID 50 | PF03583.15 |
| unknown | dipeptidylpeptidase | SEQ ID 20 | SEQ ID 50 | PF02230.17 |
| yuxL | putative acylaminoacyl-pep- | SEQ ID 21 | SEQ ID 51 | PF02230.17 |
| | tidase | | | |
| unknown | phage putative hydrolase | SEQ ID 22 | SEQ ID 52 | PF01764.26 |
| | protein | | | |
| besA | trilactone hydrolase | SEQ ID 23 | SEQ ID 53 | PF02230.17 |
| unknown | rhamnogalacturonan acety- | SEQ ID 24 | SEQ ID 54 | PF13472.7 |
| | lesterase | | | |
| IchAD | lichenysin synthase | SEQ ID 25 | SEQ ID 55 | PF02230.17 |
| unknown | putative esterase | SEQ ID 26 | SEQ ID 56 | PF02230.17 |
| lipC | spore germination lipase | SEQ ID 27 | SEQ ID 57 | PF13472.7 |
| | LipC | | | |
| lipC | spore germination lipase | SEQ ID 27 | SEQ ID 57 | PF00657.23 |
| | LipC | | | |
| unknown | putative esterase/lipase | SEQ ID 28 | SEQ ID 58 | PF02230.17 |
| unknown | putative esterase/lipase | SEQ ID 28 | SEQ ID 58 | PF03583.15 |
| yrhL | putative peptidoglycan O- | SEQ ID 29 | SEQ ID 59 | PF13472.7 |
| | acetyltransferase | | | |
| yrhL | putative peptidoglycan O- | SEQ ID 29 | SEQ ID 59 | PF00657.23 |
| | acetyltransferase | | | |
| unknown | hypothetical protein | SEQ ID 30 | SEQ ID 60 | PF02230.17 |

Thus, the recombinant Bacillus cell may comprise a non-native reduction of the expression of one or more proteins having lipase and/or esterase activity, wherein the one or more proteins having lipase and/or esterase activity is selected from the group of proteins comprising an amino acid sequence having at least 70%, at least 75%, at least 80%, at least 85%, at least 90%, at least 92%, at least 95%, at least 96%, at least 97%, at least 98%, at least 99%, or even 100% to an amino acid sequence selected from the group consisting of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In one embodiment, the recombinant Bacillus cell comprises an inactivation of the yitV gene and/or YpmR gene and in addition an inactivation of one or more of the other genes listed in the Table 1.

Thus, in one embodiment the recombinant Bacillus cell comprises a non-native reduction of the expression of one or more proteins having lipase and/or esterase activity, wherein the one or more proteins having lipase and/or esterase activity is selected from the group of proteins comprising an amino acid sequence having at least 70%, at least 75%, at least 80%, at least 85%, at least 90%, at least 92%, at least 95%, at least 96%, at least 97%, at least 98%, at least 99%, or even 100% to an amino acid sequence selected from the group consisting of SEQ ID NO: 2 and/or 10, and comprises a non-native reduction of the expression of one or more proteins having lipase and/or esterase activity, wherein the one or more proteins having lipase and/or esterase activity is selected from the group of proteins comprising an amino acid sequence having at least 70%, at least 75%, at least 80%, at least 85%, at least 90%, at least 92%, at least 95%, at least 96%, at least 97%, at least 98%, at least 99%, or even 100% to an amino acid sequence selected from the group consisting of SEQ ID NO: 1, 3, 4, 5, 6, 7, 8, 9, 11, 12, 13, 14, 15, 16, 17, 18, 19, 20, 21, 22, 23, 24, 25, 26, 27, 28, 29, or 30.

### Host Cells

Various host cells can be used for the production of compounds of interest as described herein. Host cells comprising the genetic constructs described herein can be obtained by one of the methods described herein for introducing the polynucleotides into such host cells. In the methods of the present invention, the bacterial host cell is a Bacillus cell.

The Bacillus cell of the present invention has been modified to have a reduced expression of the endogenous YitV and/or YpmR protein.

The reduction of the expression of the YitV and/or YpmR protein can be achieved by various means known to the skilled person in the art. The reduction of the expression of the YitV and/or YpmR protein can be achieved by a non-native reduced transcription or non-native reduced translation.

In one embodiment, the reduction of expression of the YitV and/or YpmR protein is achieved by inactivation of the gene coding for the YitV and/or YpmR protein. Preferably, the reduction of the expression of the YitV and/or YpmR protein is achieved by a complete or partial deletion of the yitV and/or ypmR gene. Alternatively, the reduction of the expression of the YitV and/or YpmR protein is achieved by a partial deletion of the promoter region of the gene coding for the YitV and/or YpmR protein.

The inactivation of a gene as referred to herein may be achieved by any method deemed appropriate, i.e. that a gene encoding region and/or control sequence of a gene is entirely or partially modified by means such as deletion, insertion, substitution of one or more nucleotides or applied mutagenesis by chemical agents (e.g. NTG, EMS) or UV radiation followed by screening and selection resulting in functional inactivation, i.e. absence or decrease in expression and/or absence or decrease of enzyme activity of the encoded polypeptide. In simple terms, a “deletion” is defined as a change in either nucleotide or amino acid sequence in which one or more nucleotides or amino acid residues, respectively, have been removed (i.e., are absent). Thus, a deletion host cell has fewer nucleotides or amino acids than the respective parent host cell. The absence or decrease of enzymatic activity can be detected by measuring the absence or decreased specific enzyme activity of the corresponding polypeptide having enzyme activity (e.g. at least 25% decrease, at least 50% decrease, at least 75% decrease, at least 85% decrease, at least 95% decrease, or preferable 100% decrease). The inactivation can also be measured by the absence or decrease of corresponding mRNA of the host cell (e.g. at least 25% decrease, at least 50% decrease, at least 75% decrease, at least 85% decrease, at least 95% decrease, or preferable 100% decrease) with techniques known in the art. Inactivation of a particular gene of interest, or genomic region of interest can be generated by methods known in the art, for example, deletion/insertion/substitution by homologous recombination, gene silencing or applied mutagenesis

Gene silencing can be achieved by introducing into the bacterial host cell antisense expression constructs that result in antisense RNAs complementary to the mRNA of the gene or genes targeted for inactivation respectively, thereby inhibiting expression of said genes. Gene silencing might also be achieved by introducing into the bacterial host cell siRNA expression constructs. Alternatively, the expression of said genes can be inhibited by blocking transcriptional initiation or transcriptional elongation through the mechanism of CRISPR-inhibition (e.g., as described in WO18009520).

Gene inactivation can be achieved by homologous recombination, i.e. an incoming DNA molecule comprises sequences that are homologous to the 5′ and 3′ flanking sequences of the target sequence on the chromosome of the host cell (e.g. Bacillus) to be inactivated. Subsequently the sequence between said flanking sequences is replaced by the homologous sequences of the incoming DNA molecule in the process of homologous recombination, i.e. the sequence is deleted from the chromosome. Likewise “gene integration”, i.e. a DNA sequence such as a gene expression cassette with or without a selectable marker, can be integrated into the chromosome of the bacterial host cell by homologous recombination. Hence. The DNA sequence to be integrated is flanked by DNA sequences that are homologous to the 5′ and 3′ flanking sequences on the chromosome. It is understood in terms of the invention that gene integration can also combine gene integration and gene deletion in one step, i.e. a DNA sequence on the chromosome is replaced by the incoming DNA sequence for gene integration.

Homologous recombination can be achieved by two different methods known in the art: By two consecutive rounds of homologous recombination (Campbell recombination) with circular plasmid DNA, e.g. based on the well-known temperature sensitive plasmid pE194 (Nahrstedt et al., Strain development in Bacillus licheniformis: construction of biologically contained mutants deficient in sporulation and DNA repair. J Biotechnol. 2005 Sep. 29; 119(3):245-54).

The integration of the deletion plasmid containing an incoming DNA molecule comprising sequences that are homologous to the 5′ and 3′ flanking sequences of the target sequence on the chromosome is achieved by a first homologous recombination (Campbell recombination) with the first homologous region under selective conditions for the selectable marker and cultivation at the non-permissive temperature, i.e. that blocks plasmid replication. The second homologous recombination with the second homologous region is achieved by removal of selective pressure and cultivation at the permissive temperature, i.e. plasmid replication takes place, resulting in excision of the plasmid from the chromosome.

Alternatively, a non-replicative ‘suicide’ plasmid can be used forcing the integration by selection on the selectable marker. Only cells that have integrated the plasmid into the genome by homologous recombination are able to grow under the selective conditions. Plasmid removal/excision from the chromosome is achieved with a second homologous recombination which is forced by the activation of a counterselection marker present on the plasmid.

The second method of homologous recombination refers to two homologous recombination events simultaneously taking place, also known as ‘double crossing over’ or ‘double homologous recombination. The incoming DNA sequence is linear and can be obtained by PCR, linearization of plasmid DNA or preparation of chromosomal DNA which inevitable results in fragmented linear DNA. WO0308125 uses linear DNA constructs (either linearized plasmids or PCR fragments) comprising a selectable marker flanked by the 5′ and 3′ homologous regions which are used for genomic integration via double crossing over homologous recombination. It is well understood that next to the selectable marker additional DNA, such as gene expression cassettes, when flanked by said homologous region are integrated into the chromosome of the bacterial host cell.

Homologous recombination requires DNA sequences homologous to the 5′ and 3′ flanking sequences of the target sequence on the chromosome of the host cell of sufficient size, hence should contain a sufficient number of nucleic acid such as 100 to 1,500 base pairs, preferably 400 to 1,500 base pairs, and most preferably 800 to 1,500 base pairs, which have a high degree of identity to the corresponding target sequence to enhance the probability of homologous recombination (Dubnau, 1993, Genetic exchange and homologous recombination. In Bacillus subtilis and Other Gram-positive Bacteria, p. 555-584. Edited by A. I. Sonenshein, J. A. Hoch & R. Losick, Washington DC, American Society for Microbiology; Michel and Ehrlich, 1984, The EMBO Journal, vol. 3, pp. 2879-2884).

Gene inactivation by deletion/insertion/substitution can also be achieved by CRISPR/Cas9 genome editing technologies where the CRISPR cutting properties could be used to disrupt genes in almost any organism's genome with unprecedented ease (Mali P, et al (2013) Science. 339(6121):819-823; Cong L, et al (2013) Science 339(6121)). Recently it became clear that providing a template for repair, e.g. homologous regions, allowed for editing the genome with nearly any desired sequence at nearly any site, transforming CRISPR into a powerful gene editing tool (WO/2014/150624, WO/2014/204728).

CRISPR-based expression systems for application in gram positive organisms such as Bacillus species based on the single-plasmid system approach, i.e. comprising the Cas9 endonuclease, the gRNA (e.g. sgRNA or crRNA/tracrRNA), repair homology sequences (donor DNA) on one single E. coli-Bacillus shuttle vector have been successfully applied (Altenbuchner, (2016): Applied and environmental microbiology 82 (17), 5421-5427; Zhou, et al. (2019): International journal of biological macromolecules 122, 329-337).

Alternatively to “directed” methods of inactivation it is understood in the scope of the invention that whole-cell mutagenesis by applying mutagenizing conditions such as exposure of the cells to UV radiation, or chemical mutagenizing chemicals such as NTG (N-methyl-N′-nitro-N-nitrosoguanidine), EMS (ethyl-methane-sulfonate), in combination with screening and/—or selection of the desired property, e.g. reduced lipase/esterase activity is a well-known approach to achieve functional inactivation.

The introduction of DNA into a host cell, in one embodiment a Bacillus cell, may, for instance, be effected by protoplast transformation (see, e.g., Chang and Cohen, 1979, Molecular General Genetics 168: 111-115), by using competent cells (see, e.g., Young and Spizizen, 1961, Journal of Bacteriology 81: 823-829, or Dubnau and Davidoff-Abelson, 1971, Journal of Molecular Biology 56: 209-221), by electroporation (see, e.g., Shigekawa and Dower, 1988, Biotechniques 6: 742-751), or by conjugation (see, e.g., Koehler and Thorne, 1987, Journal of Bacteriology 169: 5271-5278). Specific transformation protocols are known in the art for various types of host cells (see, e.g., for E. coli protoplast transformation see Hanahan, 1983, J. Mol. Biol. 166: 557-580).

In a preferred embodiment, the Bacillus cell comprises a non-native complete or partial deletion of the gene coding for the YitV and/or YpmR protein or the Bacillus cell comprises a non-native complete or partial deletion of the promoter region of the gene coding for the YitV and/or YpmR protein resulting in the non-native reduction of the YitV and/or YpmR protein expression. Preferably, the Bacillus cell comprises a non-native complete deletion of the gene coding for the YitV and/or YpmR protein or the Bacillus cell comprises a non-native complete deletion of the promoter region of the gene coding for the YitV and/or YpmR protein resulting in the non-native reduction of the YitV and/or YpmR protein expression.

Preferably the Bacillus cell comprises a full or partial deletion of or one or more point mutations in the yitV and/or ypmR gene resulting in a reduced expression of the YitV and/or YpmR protein. Preferably, the inactivated yitV gene comprises a nucleotide sequence having at least 70%, at least 75%, at least 80%, at least 85%, at least 90%, at least 92%, at least 95%, at least 96%, at least 97%, at least 98%, at least 99%, or even 100% identity with SEQ ID NO: 32.

Preferably, the inactivated ypmR gene comprises a nucleotide sequence having at least 70%, at least 75%, at least 80%, at least 85%, at least 90%, at least 92%, at least 95%, at least 96%, at least 97%, at least 98%, at least 99%, or even 100% identity with SEQ ID NO: 40.

Preferably, the expression of the YitV protein is reduced by an inactivation of the yitV gene, wherein the yitV gene comprises a nucleotide sequence having at least 80%, at least 85%, at least 90%, at least 92%, at least 95%, at least 96%, at least 97%, at least 98%, at least 99%, or even 100% identity with SEQ ID NO: 32.

Preferably, the expression of the YpmR protein is reduced by an inactivation of the ypmR gene, wherein the ypmR gene comprises a nucleotide sequence having at least 80%, at least 85%, at least 90%, at least 92%, at least 95%, at least 96%, at least 97%, at least 98%, at least 99%, or even 100% identity with SEQ ID NO: 40.

However, also other genes coding for enzymes with lipase and/or esterase activity could be reduced in their expression in the Bacillus cell, preferably in addition to the yitV and/or ypmR gene. For instance, one or more of the proteins shown in Table 1 can be reduced in its expression, preferably by full or partial deletion of or by point mutations in one or more genes comprising a polynucleotide having at least 70%%, at least 75%, at least 80%, at least 85%, at least 90%, at least 92%, at least 95%, at least 96%, at least 97%, at least 98%, at least 99%, or even 100% identity with SEQ ID NO: 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60.

Bacillus cells useful in the practice of the present invention include, but are not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus methylotrophicus, Bacillus cereus Bacillus paralicheniformis, Bacillus subtilis, and Bacillus thuringiensis cells. In one embodiment, the bacterial host cell is a Bacillus amyloliquefaciens, Bacillus lentus, Bacillus licheniformis, Bacillus pumilus, Bacillus stearothermophilus or Bacillus subtilis cell. In another embodiment, the bacterial host cell is a Bacillus licheniformis cell or a Bacillus subtilis cell, in a specifically preferred embodiment a Bacillus licheniformis cell. In one embodiment, the Bacillus cell is not a Bacillus subtilis cell.

For industrial fermentation processes, the bacterial host cell may be genetically modified to meet the needs of highest product purity and regulatory requirements. It is therefore in scope of the invention to use Bacillus production hosts that may additionally contain modifications, e.g., deletions or disruptions, of other genes that may be detrimental to the production, recovery or application of a polypeptide of interest. In one embodiment, a bacterial host cell is a protease-deficient cell. The bacterial host cell, e.g., Bacillus cell, preferably comprises a disruption or deletion of extracellular protease genes including but not limited to aprE, mpr, vpr, bpr, and/or epr. Further preferably the bacterial host cell does not produce spores. Further preferably the bacterial host cell, e.g., a Bacillus cell, comprises a disruption or deletion of spol/AC, sigE, and/or sigG. Further, preferably the bacterial host cell, e.g., Bacillus cell, comprises a disruption or deletion of one of the genes involved in the biosynthesis of surfactin, e.g., srfA, srfB, srfC, and/or srfD, see, for example, U.S. Pat. No. 5,958,728. It is also preferred that the bacterial host cell comprises a disruption or deletion of one of the genes involved in the biosynthesis of polyglutamic acid. Other genes, including but not limited to the amyE gene, which are detrimental to the production, recovery or application of a polypeptide of interest may also be disrupted or deleted.

In one embodiment, the Bacillus cell comprises a selectable marker. The selectable marker can be antibiotic resistance markers such as ampicillin, kanamycin, erythromycin, chloramphenicol or tetracycline, or an auxotrophic resistance marker.

Optionally the Bacillus cell might comprise a counterselection markers as described herein. In a preferred embodiment, the counterselection polypeptide is a polypeptide which involved in the pyrimidine metabolism. Thus, the counterselection polypeptide, such as oroP, pyrE, pyrF, upp, uses flourated analogons of intermediates in the pyrmidine metabolism, such as, 5-fluoro-orotate or 5-fluoro-uridine. Alternatively, toxins of toxin-anti-toxin systems (TA) such as the mazEF, ccdAB could be used as functional counterselection polypeptides in Bacillus (see Dong, H., Zhang, D. Current development in genetic engineering strategies of Bacillus species. Microb Cell Fact 13, 63 (2014)). In an even more preferred embodiment, the couterselection polypeptide is a cytosine deaminase, such as provided by the codBA system (Kostner D, Rachinger M, Liebl W, Ehrenreich A. Markerless deletion of putative alanine dehydrogenase genes in Bacillus licheniformis using a codBA-based counterselection technique. Microbiology. 2017; 163(11):1532-1539). Preferably, the counterselection agent is 5-fluoro-cytosine.

The Bacillus cell of the present invention preferably produces a compound of interest, preferably a protein, as described herein. Preferably, the Bacillus cell comprises a gene encoding for a protein of interest, preferably a heterologous protein of interest. Preferably the Bacillus cell is a Bacillus licheniformis cell.

### Methods of the Invention

The present invention is also directed to various methods using the improved Bacillus cell of the present invention as described herein.

### Production of a Compound of Interest

The recombinant Bacillus cells created by the methods described herein are particularly useful as host cells for the production of a compound of interest. Therefore, the present invention is also directed to a method of cultivating a recombinant Bacillus cells obtained by any of the methods described herein. In a particular embodiment, the present invention is further directed to methods of producing a compound of interest comprising: (a) cultivating the recombinant cell under conditions conducive for production of the compound of interest; and (b) optionally recovering the compound of interest from the fermentation broth.

The compound of interest may or may not be further purified from the fermentation broth. Thus, in one embodiment, the present invention refers to a fermentation broth comprising a compound of interest obtained by a fermentation process as described herein.

In one embodiment, the present invention is directed to a method of producing a compound of interest comprising the steps of
a) cultivating a Bacillus cell in the fermentation medium described herein for a time and under conditions suitable for the Bacillus cell to produce the compound of interest; and
b) optionally, purifying the compound of interest from the fermentation broth.

The compound of interest can be produced by a Bacillus cell expressing one or more polynucleotides native or heterologous to the cell. Preferably the one or more polynucleotides native or heterologous to the cell codes for a protein of interest.

Thus, preferred is a method for producing a compound of interest, preferably a polypeptide of interest, comprising the steps of
a) providing a modified Bacillus host cell comprising an inactivation of the yitV and/or ypmR gene,
b) introducing into said modified Bacillus host cell an expression cassette for a compound of interest, preferably a polypeptide of interest,
c) cultivating the host cell under conditions which allow for the expression of the compound of interest, and
d) optionally isolating the compound of interest from the cultivation medium.

Particularly preferred is a method for producing a compound of interest, preferably a polypeptide of interest, preferably an enzyme, comprising the steps of
a) providing a modified Bacillus host cell comprising an inactivation of the yitV and ypmR gene,
b) introducing into said modified Bacillus host cell an expression cassette for a compound of interest, preferably a polypeptide of interest,
c) cultivating the host cell under conditions which allow for the expression of the compound of interest, and
d) optionally isolating the compound of interest from the cultivation medium.

In a particular embodiment, the present invention is further directed to methods of producing a native or foreign polypeptide comprising: (a) cultivating the recombinant cell under conditions conducive for production of the polypeptide; and (b) optionally recovering the polypeptide.

Thus, preferred is a method for producing a polypeptide of interest, preferably an enzyme, comprising the steps of
a) providing a modified Bacillus host cell comprising an inactivation of the yitV and/or ypmR gene, preferably an inactivation of the yitV and the ypmR gene,
b) introducing into said modified Bacillus host cell an expression cassette for a compound of interest, preferably a polypeptide of interest,
c) cultivating the host cell under conditions which allow for the expression of the compound of interest, and
d) optionally isolating the compound of interest from the cultivation medium.

Thus, the present invention is also directed to a method for producing a heterologous protein of interest in a bacterial cell comprising the step of cultivating the bacterial host cell obtained by any of the methods described herein for a time and under conditions sufficient to produce the heterologous protein. The bacterial host cells are cultivated in a nutrient medium suitable for production of a polypeptide of interest using methods known in the art. For example, the cell may be cultivated by shake flask cultivation, small-scale or largescale fermentation (including continuous, batch, fed-batch, or solid state fermentations) in laboratory or industrial fermenters performed in a suitable medium and under conditions allowing the polypeptide of interest to be expressed and/or isolated. In one embodiment, the cultivation of the bacterial host cell is by fermentation in industrial scale, e.g. WO2020169563A1 The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). The protein of interest can accumulate in the cell or can be secreted outside of the cell. The secreted polypeptide of interest can be recovered directly from the medium. The polypeptide of interest may be detected using methods known in the art that are specific for the polypeptide. These detection methods may include use of specific antibodies, high performance liquid chromatography, capillary chromatography, formation of an enzyme product, disappearance of an enzyme substrate, or SDS-PAGE. For example, an enzyme assay may be used to determine the activity of the enzyme. Procedures for determining enzyme activity are known in the art for many enzymes (see, for example, D. Schomburg and M. Salzmann (eds.), Enzyme Handbook, Springer-Verlag, New York, 1990).

The resulting protein of interest may be isolated by methods known in the art. For example, a protein of interest may be isolated from the fermentation broth by conventional procedures including, but not limited to, centrifugation, filtration, extraction, spray-drying, evaporation, or precipitation. Depending on the expression construct used, the protein of interest can be secreted into the fermentation broth or can remain inside the host cell. In case of the latter, the protein of interest can be recovered from the fermentation broth by applying a step where the cells are lysed. The isolated polypeptide may then be further purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989). The purified polypeptide may then be concentrated by procedures known in the art including, but not limited to, ultrafiltration and evaporation, in particular, thin film evaporation. In another embodiment, the protein of interest is not purified from the fermentation broth. In a specific embodiment, the protein of interest is not secreted in the fermentation broth and not recovered from the fermentation broth.

### Compound of Interest, Preferably Protein of Interest

The host cell of the present invention shall further comprise an expression cassette for the production of a compound of interest, preferably a protein of interest.

Compounds of interest maybe polymers, preferably hyaluronic acidy, preferably as described in (WO2005098016), or polyglutamic acid, preferably as described in EP2196534, or maybe vitamins, preferably vitamin B5, preferably as described in WO2010018169, or riboflavin, preferably as described in WO2017036903, or may be polypeptides, preferably enzymes.

Preferably, the compound of interest, preferably protein of interest, may be secreted by the Bacillus host cell or may be produced intracellularly.

In one embodiment, the compound of interest is a protein of interest. Preferably, the protein of interest is encoded by a gene introduced into the Bacillus cell by means of recombinant DNA technology. Preferably, the protein of interest is heterologous to the Bacillus cell. Preferably, the protein of interest is an enzyme. The enzyme may be, but is not limited to, a detergent enzyme and an enzyme suitable for human and/or animal nutrition. In one preferred embodiment, the enzyme is an enzyme suitable for the use in human nutrition. In one embodiment, the enzyme is classified as an oxidoreductase (EC 1), a transferase (EC 2), a hydrolase (EC 3), a lyase (EC 4), an isomerase (EC 5), or a Ligase (EC 6) (EC-numbering according to Enzyme Nomenclature, Recommendations (1992) of the Nomenclature Committee of the International Union of Biochemistry and Molecular Biology including its supplements published 1993-1999). In one embodiment, the protein of interest is a protein conferring resistance to antibiotics to a host cell.

In another embodiment, the enzyme is a hydrolase (EC 3), in one embodiment a glycosidase (EC 3.2) or a peptidase (EC 3.4). In one embodiment, enzymes selected from the group consisting of an amylase (in particular an alpha-amylase (EC 3.2.1.1)), a cellulase (EC 3.2.1.4), a lactase (EC 3.2.1.108), a mannanase (EC 3.2.1.25), a lipase (EC3.1.1.3), a phytase (EC 3.1.3.8), a nuclease (EC 3.1.11 to EC 3.1.31), and a protease. In one embodiment, the enzyme is selected from the group consisting of oxidoreductase, transferase, hydrolase, lyase, isomerase, ligase, aminopeptidase, amylase, asparaginase, carbohydrase, carboxypeptidase, catalase, cellulase, chitinase, cutinase, cyclodextrin glycosyltransferase, deoxyribonuclease, esterase, alpha-galactosidase, betagalactosidase, glucoamylase, alpha-glucosidase, beta-glucosidase, hyaluronic acid synthase, invertase, laccase, lipase, mannosidase, mutanase, oxidase, a pectinolytic enzyme, peroxidase, phytase, polyphenoloxidase, protease, ribonuclease, transglutaminase, and orxylanase. In particular an enzyme selected from the group consisting of amylase, protease, mannanase, lactase, and cellulase, preferably protease, amylase, lactase and mannanse, in a specific embodiment amylase or protease, in one embodiment, a serine protease (EC 3.4.21). In another embodiment the enzyme is a subtilisin protease.

For producing a protein of interest, the Bacillus cell can comprise an expression construct comprising one or more control sequences required for expression of the polynucleotide. Such control sequences include, but are not limited to, promoter sequence, 5′-UTR (also called leader sequence), ribosomal binding site (RBS, shine dalgarno sequence), 3′-UTR, and transcription start and stop sites. In one embodiment, the control sequences may be provided with linkers for the purpose of introducing specific restriction sites facilitating ligation of the control sequences with the polynucleotide to be expressed. The expression of the protein of interest maybe plasmid-based expression or the gene coding for the protein of interest may be integrated into the genome of the Bacillus cell.

The nucleic acid construct comprises a suitable transcription start and terminator sequence. Any transcription start or terminator that is functional in the host cell of choice may be used in the present invention.

In one embodiment the nucleic acid construct comprises a suitable UTR (untranslated region) sequence. In one embodiment, the nucleic acid construct described herein comprises a 5′UTR and/or a 3′UTR sequence. In one embodiment, the one or more control sequence of the nucleic acid construct comprises a 5′UTR, also referred to as leader sequence. In another one embodiment, the one or more control sequence of the nucleic acid construct comprises a 5′UTR sequence comprising a ribosome-binding site also referred to as a shine-dalgarno sequence. Any leader sequence that is functional in the host cell of choice may be used in the present invention. The UTR can be natural or artificial.

In a preferred embodiment, the replication of a plasmid shall be independent of the replication of the chromosome of the bacterial host cell. For autonomous replication, the expression vector may further comprise an origin of replication enabling the vector to replicate autonomously in the host cell in question. Bacterial origins of replication include but are not limited to the origins of replication of plasmids pBR322, pUC19, pSC101, pACYC177, and pACYC184 permitting replication in E. coli (Sambrook, J. and Russell, D. W. Molecular cloning. A laboratory manual, 3rd ed, Cold Spring Harbor Laboratory Press, Cold Spring Harbor, N Y. 2001; Cohen, S. N., Chang, A. C. Y., Boyer, H. W., & Helling, R. B. (1973). Construction of Biologically Functional Bacterial Plasmids In Vitro. Proceedings of the National Academy of Sciences of the United States of America, 70(11), 3240-3244), and pUB110, pC194, pE194, pTB19, pAMβ1, and pTA1060 permitting replication in Bacillus (Janniere, L., Bruand, C., and Ehrlich, S. D. (1990). Structurally stable Bacillus subtilis cloning vectors. Gene 87, 53-6; Ehrlich, S. D., Bruand, C., Sozhamannan, S., Dabert, P., Gros, M. F., Janniere, L., and Gruss, A. (1991). Plasmid replication and structural stability in Bacillus subtilis. Res. Microbiol. 142, 869-873), and pE194 (Dempsey, L. A. and Dubnau, D. A. (1989). Localization of the replication origin of plasmid pE194. J. Bacteriol. 171, 2866-2869). The origin of replication may be one having a mutation to make its function temperature-sensitive in the host cell (see, e.g., Ehrlich, 1978, Proceedings of the National Academy of Sciences USA 75:1433-1436).

The copy number of a plasmid is defined as the average number of plasmids per bacterial cell or per chromosome under normal growth conditions. Moreover, there are different types of replication origins that result in different copy numbers in the bacterial host. The plasmid replicon pBS72 (accession number AY102630.1) and the plasmids pTB19 and derivatives pTB51, pTB52 confer low copy number with 6 copies and 1 to 8 copies, respectively, within Bacillus cells whereas plasmids pE194 (accession number V01278.1) and pUB110 (accession number M19465.1)/pBC16 (accession number U32369.1) confer low-medium copy number with 14-20 and medium copy number with 30-50 copies per cell, respectively. Plasmid pE194 was analyzed in more detail (Villafane, et al (1987): J. Bacteriol. 169(10), 4822-4829) and several pE194—cop mutants described having high copy numbers within Bacillus ranging from 85 copies to 202 copies. Moreover, plasmid pE194 is temperature sensitive with stable copy number up to 37° C., however abolished replication above 43° C. In addition, it exists a pE194 variant referred to as pE194ts with two point mutations within the cop-repF region (nt 1235 ad nt 1431) leading to a more drastic temperature sensitivity—stable copy number up to 32° C., however only 1 to 2 copies per cell at 37° C.

In one embodiment, the vectors contain one or more selectable markers that permit easy selection of transformed cells. A selectable marker is a gene encoding a product, which provides for biocide resistance, resistance to heavy metals, prototrophy to auxotrophs, and the like. Bacterial selectable markers include but are not limited to the dal genes from Bacillus subtilis or Bacillus licheniformis, or markers that confer antibiotic resistance such as ampicillin, kanamycin, erythromycin, chloramphenicol or tetracycline resistance. Furthermore, selection may be accomplished by co-transformation, e.g., as described in WO91/09129, where the selectable marker is on a separate vector.

A promoter for expressing the protein of interest or the selectable marker can be an “inducer-dependent promoter” or an “inducer-independent promoter” comprising constitutive promoters or promoters which are under the control of other cellular regulating factors.

The person skilled in the art is capable to select suitable promoters for expressing the third alanine racemase and the polypeptide of interest. For example, the polynucleotide encoding the polypeptide of interest is, preferably, operably linked to an “inducer-dependent promoter” or an “inducer-independent promoter”. Further, the polynucleotide encoding the third alanine racemase is, preferably, operably linked to an “inducer-independent promoter”, such as a constitutive promoter.

An “inducer dependent promoter” is understood herein as a promoter that is increased in its activity to enable transcription of the gene to which the promoter is operably linked upon addition of an “inducer molecule” to the fermentation medium. Thus, for an inducer-dependent promoter, the presence of the inducer molecule triggers via signal transduction an increase in expression of the gene operably linked to the promoter. The gene expression prior activation by the presence of the inducer molecule does not need to be absent, but can also be present at a low level of basal gene expression that is increased after addition of the inducer molecule. The “inducer molecule” is a molecule which presence in the fermentation medium is capable of affecting an increase in expression of a gene by increasing the activity of an inducer-dependent promoter operably linked to the gene. Preferably, the inducer molecule is a carbohydrate or an analog thereof. In one embodiment, the inducer molecule is a secondary carbon source of the Bacillus cell. In the presence of a mixture of carbohydrates cells selectively take up the carbon source that provide them with the most energy and growth advantage (primary carbon source). Simultaneously, they repress the various functions involved in the catabolism and uptake of the less preferred carbon sources (secondary carbon source). Typically, a primary carbon source for Bacillus is glucose and various other sugars and sugar derivates being used by Bacillus as secondary carbon sources. Secondary carbon sources include e.g. mannose or lactose without being restricted to these.

Examples of inducer dependent promoters are given in the table below by reference to the respective operon:

| Operon | Regulator a) | Type b) | Inducer | Organism |
|---|---|---|---|---|
| sacPA | SacT | AT | sucrose | B.subtilis |
| sacB | SacY | AT | sucrose | B.subtilis |
| bgl PH | LicT | AT | β-glucosides | B.subtilis |
| licBCAH | LicR | A | oligo-β-gluco- | B.subtilis |
| | | | sides | |
| levDEFG sacL | LevR | A | fructose | B.subtilis |
| mtIAD | MtIR | A | mannitol | B.subtilis |
| manPA-yjdF | ManR | A | mannose | B.subtilis |
| manR | ManR | A | mannose | B.subtilis |
| bgIFB bgIG | BgIG | AT | β-glucosides | E.coli |
| lacTEGF | LacT | AT | lactose | L.casei |
| lacZYA | lacl | R | Allolactose; | E.coli |
| | | | IPTG (Isopropyl | |
| | | | β-D-1-thiogalac- | |
| | | | topyranoside) | |
| araBAD | araC | AR | L-arabinose | E.coli |
| xylAB | XyIR | R | Xylose | B.subtilis |

a: transcriptional regulator protein
b: A: activator
AT: antiterminator
R: repressor
AR: activator/repressor

In contrast thereto, the activity of promoters that do not depend on the presence of an inducer molecule (herein called ‘inducer-independent promoters’) are either constitutively active or can be increased regardless of the presence of an inducer molecule that is added to the fermentation medium.

Constitutive promoters are independent of other cellular regulating factors and transcription initiation is dependent on sigma factor A (sigA). The sigA-dependent promoters comprise the sigma factor A specific recognition sites ‘-35’-region and ‘-10’-region.

Preferably, the ‘inducer-independent promoter’ sequence is selected from the group consisting of constitutive promoters not limited to promoters Pveg, PlepA, PserA, PymdA, Pfba and derivatives thereof with different strength of gene expression (Guiziou et al, (2016): Nucleic Acids Res. 44(15), 7495-7508), the aprE promoter, the bacteriophage SPO1 promoters P4, P5, P15 (WO15118126), the cryIIIA promoter from Bacillus thuringiensis (WO9425612), the amyQ promoter from Bacillus amyloliquefaciens, the amyL promoter and promoter variants from Bacillus licheniformis (U.S. Pat. No. 5,698,415) and combinations thereof, or active fragments or variants thereof, preferably an aprE promoter sequence.

Further optionally the expression construct comprises a 5′UTR. This is a transcribed but not translated region downstream of the −1 promoter position. Such untranslated region for example should contain a ribosome binding site to facilitate translation in those cases where the target gene codes for a peptide or polypeptide.

With respect to the 5′UTR the invention in particular teaches to combine the promoter of the present invention with a 5UTR comprising one or more stabilising elements. This way the mRNAs synthesized from the promoter region may be processed to generate mRNA transcript with a stabilizer sequence at the 5′ end of the transcript. Preferably such a stabilizer sequence at the Send of the mRNA transcripts increases their half-life as described by Hue et al, 1995, Journal of Bacteriology 177: 3465-3471. Suitable mRNA stabilizing elements are those described in
WO08148575, preferably SEQ ID NO. 1 to 5 of WO08140615, or fragments of these sequences which maintain the mRNA stabilizing function, and in
WO08140615, preferably Bacillus thuringiensis CryIIIA mRNA stabilising sequence or bacteriophage SP82 mRNA stabilising sequence, more preferably a mRNA stabilising sequence according to SEQ ID NO. 4 or 5 of WO08140615, more preferably a modified mRNA stabilising sequence according to SEQ ID NO. 6 of WO08140615, or fragments of these sequences which maintain the mRNA stabilizing function.

Preferred mRNA stabilizing elements are selected from the group consisting of aprE, grpE, cotG, SP82, RSBgsiB, CryIIIA mRNA stabilizing elements, or according to fragments of these sequences which maintain the mRNA stabilizing function. A preferred mRNA stabilizing element is the grpE mRNA stabilizing element (corresponding to SEQ ID NO. 2 of WO08148575).

The 5UTR also preferably comprises a modified rib leader sequence located downstream of the promoter and upstream of an ribosome binding site (RBS). In the context of the present invention a rib leader is herewith defined as the leader sequence upstream of the riboflavin biosynthetic genes (rib operon) in a Bacillus cell, more preferably in a Bacillus subtilis cell. In Bacillus subtilis, the rib operon, comprising the genes involved in riboflavin biosynthesis, include ribG (ribD), ribB (ribE), ribA, and ribH genes. Transcription of the riboflavin operon from the rib promoter (Prib) in B. subtilis is controlled by a riboswitch involving an untranslated regulatory leader region (the rib leader) of almost 300 nucleotides located in the 5-region of the rib operon between the transcription start and the translation start codon of the first gene in the operon, ribG. Suitable rib leader sequences are described in WO2015/1181296, in particular pages 23-25, incorporated herein by reference.

In a preferred embodiment, the present invention is directed to a method of producing a protein of interest comprising the steps of
a) cultivating a Bacillus licheniformis cell comprising a reduced expression of the YitV and/or YpmR protein in the fermentation medium described herein for a time and under conditions suitable for the Bacillus cell to produce the protein of interest; and
b) optionally, purifying the protein of interest from the fermentation broth.

In a preferred embodiment, the present invention is directed to a method of producing a protein of interest comprising the steps of
a) cultivating a Bacillus licheniformis cell comprising a deletion of the yitV and/or ypmR gene in the fermentation medium described herein for a time and under conditions suitable for the Bacillus cell to produce the protein of interest; and
b) optionally, purifying the protein of interest from the fermentation broth.

In a particularly preferred embodiment, the present invention is directed to a method of producing an enzyme comprising the steps of
a) cultivating a Bacillus licheniformis cell comprising a deletion of the yitV and of the ypmR gene in the fermentation medium described herein for a time and under conditions suitable for the Bacillus cell to produce the enzyme; and
b) optionally, purifying the enzyme from the fermentation broth.

Method for reducing the formation of malodour or off-flavour In another embodiment, the present invention is directed to a method for reducing the formation of malodour or off-flavour in a composition comprising a compound of interest obtained by fermentation of a Bacillus cell comprising the fermentation of a Bacillus cell as described herein capable of producing a compound of interest, purifying the compound of interest from the fermentation broth and providing a composition comprising the purified compound of interest.

In a preferred embodiment, the present invention is directed to a method for reducing the formation of malodour or off-flavour in a composition comprising a compound of interest obtained by fermentation of a Bacillus cell comprising the steps of
a) cultivating a Bacillus cell comprising reduced expression of the YitV and/or YpmR protein in a fermentation medium described herein for a time and under conditions suitable for the Bacillus cell to produce the protein of interest; and
b) purifying the protein of interest from the fermentation broth;
c) providing a composition comprising the protein of interest of step (b).

In a preferred embodiment, the present invention is directed to a method for reducing the formation of malodour or off-flavour in a composition comprising a compound of interest obtained by fermentation of a Bacillus licheniformis cell comprising the steps of
a) cultivating a Bacillus licheniformis cell comprising reduced expression of the YitV and/or YpmR protein in a fermentation medium described herein for a time and under conditions suitable for the Bacillus cell to produce the protein of interest; and
b) purifying the protein of interest from the fermentation broth;
c) providing a composition comprising the protein of interest of step (b).

A further embodiment of the invention is a method for reducing the lipase and/or esterase activity in a Bacillus cell comprising the inactivation of the yitV and/or ypmR gene in the Bacillus cell, preferably the inactivation of the yitV and ypmR gene in the Bacillus cell, preferably in a Bacillus licheniformis cell.

An additional embodiment of the present invention is a method for reducing the lipase and/or esterase activity of a compound of interest, preferably a protein of interest, preferably an enzyme, produced by a Bacillus cell comprising the steps of
a) providing a modified Bacillus host cell comprising an inactivation of the yitV and/or ypmR gene,
b) introducing into said modified Bacillus host cell an expression cassette for a compound of interest, preferably a polypeptide of interest,
c) cultivating the host cell under conditions which allow for the expression of the compound of interest, and
d) optionally isolating the compound of interest from the cultivation medium.

An further embodiment of the present invention is a method for reducing the formation of malodour or off-flavor in a composition comprising a compound of interest, preferably a polypeptide of interest, preferably an enzyme, produced by a Bacillus cell, preferably a Bacillus licheniformis cell, comprising the steps of
a) providing a modified Bacillus host cell comprising an inactivation of the yitV and/or ypmR gene,
b) introducing into said modified Bacillus host cell an expression cassette for a compound of interest, preferably a polypeptide of interest,
c) cultivating the host cell under conditions which allow for the expression of the compound of interest, and
d) optionally isolating the compound of interest from the cultivation medium.

Preferred is a method for reducing the formation of malodour or off-flavor in a composition comprising a compound of interest, preferably a polypeptide of interest, preferably an enzyme, produced by a Bacillus cell, preferably Bacillus licheniformis cell, comprising the steps of a) providing a modified Bacillus host cell comprising an inactivation of the yitV and ypmR gene,
b) introducing into said modified Bacillus host cell an expression cassette for a compound of interest, preferably a polypeptide of interest,
c) cultivating the host cell under conditions which allow for the expression of the compound of interest, and
d) optionally isolating the compound of interest from the cultivation medium.

### Composition Comprising the Compound of Interest

In another embodiment, the present invention is directed to a composition comprising the purified compound of interest, preferably a protein of interest, obtained from a fermentation broth of the Bacillus cells comprising a reduced expression no of the YitV and/or YpmR protein. Compared to a composition obtained with a compound of interest obtained from a fermentation broth of Bacillus cells not according to the invention, the composition comprises a reduced level of YitV and/or YpmR protein, preferably a reduced level of YitV and YpmR protein.

In a preferred embodiment, the composition comprising the compound of interest obtained is obtained by a method comprising the steps of
a) cultivating a Bacillus cell comprising reduced expression of the YitV and/or YpmR protein as described herein in a fermentation medium for a time and under conditions suitable for the Bacillus cell to produce the protein of interest; and
b) purifying the protein of interest from the fermentation broth;
c) providing a composition comprising the protein of interest of step (b).

In a preferred embodiment, the composition comprising the compound of interest obtained is obtained by a method comprising the steps of
a) cultivating a Bacillus licheniformis cell comprising reduced expression of the YitV and/or YpmR protein as described herein in a fermentation medium for a time and under conditions suitable for the Bacillus cell to produce the protein of interest; and
b) purifying the protein of interest from the fermentation broth;
c) providing a composition comprising the protein of interest of step (b).

Preferred is a composition comprising a compound of interest, preferably a protein of interest, preferably an enzyme, with reduced contamination with lipase and/or esterase activity obtained by a method comprising the steps of
a) providing a modified Bacillus host cell comprising an inactivation of the yitV and/or ypmR gene,
b) introducing into said modified Bacillus host cell an expression cassette for a compound of interest, preferably a polypeptide of interest,
c) cultivating the host cell under conditions which allow for the expression of the compound of interest, and
d) optionally isolating the compound of interest from the cultivation medium.

Particularly preferred is a composition comprising a compound of interest, preferably a protein of interest, with reduced contamination with lipase and/or esterase activity obtained by a method comprising the steps of
a) providing a modified Bacillus host cell comprising an inactivation of the yitV and ypmR gene,
b) introducing into said modified Bacillus host cell an expression cassette for a compound of interest, preferably a polypeptide of interest,
c) cultivating the host cell under conditions which allow for the expression of the compound of interest, and
d) optionally isolating the compound of interest from the cultivation medium.

Further preferred is a composition comprising a compound of interest, preferably a protein of interest, with reduced contamination with YitV and/or YpmR protein obtained by a method comprising the steps of
a) providing a modified Bacillus host cell comprising an inactivation of the yitV and/or ypmR gene,
b) introducing into said modified Bacillus host cell an expression cassette for a compound of interest, preferably a polypeptide of interest,
c) cultivating the host cell under conditions which allow for the expression of the compound of interest, and
d) optionally isolating the compound of interest from the cultivation medium.

Further preferred is a composition comprising a compound of interest, preferably a protein of interest, with reduced contamination with YitV and YpmR protein obtained by a method comprising the steps of
a) providing a modified Bacillus host cell comprising an inactivation of the yitV and ypmR gene,
b) introducing into said modified Bacillus host cell an expression cassette for a compound of interest, preferably a polypeptide of interest,
c) cultivating the host cell under conditions which allow for the expression of the compound of interest, and
d) optionally isolating the compound of interest from the cultivation medium.

Particularly preferred is a composition comprising a protein of interest, preferably an enzyme, with reduced contamination with YitV and YpmR protein obtained by a method comprising the steps of
a) providing a modified Bacillus host cell, preferably a Bacillus licheniformis host cell, comprising an inactivation of the yitV and ypmR gene,
b) introducing into said modified Bacillus host cell an expression cassette for a compound of interest, preferably a polypeptide of interest,
c) cultivating the host cell under conditions which allow for the expression of the compound of interest, and
d) optionally isolating the compound of interest from the cultivation medium.

In one embodiment, the composition comprising the compound of interest is for laundry detergents, dish washing detergents, and cleaning products for homes, industry, vehicle care, baking, human or animal feed, pulp and paper processing, starch processing, and ethanol production. In a preferred embodiment the comprising the compound of interest is a detergent composition comprising one or more detergents. In a further preferred embodiment, the composition comprising the compound of interest is a food or feed composition, preferably a dairy composition.

### EXAMPLES

The following examples only serve to illustrate the invention. The numerous possible variations that are obvious to a person skilled in the art also fall within the scope of the invention.

Unless otherwise stated the following experiments have been performed by applying standard equipment, methods, chemicals, and biochemicals as used in genetic engineering and fermentative production of chemical compounds by cultivation of microorganisms. See also Sambrook et al. (Sambrook, J. and Russell, D. W. Molecular cloning. A laboratory manual, 3rd ed, Cold Spring Harbor Laboratory Press, Cold Spring Harbor, N Y. 2001) and Chmiel et al. (Bioprocesstechnik 1. Einführung in die Bioverfahrenstechnik, Gustav Fischer Verlag, Stuttgart, 1991).

Electrocompetent Bacillus licheniformis Cells and Electroporation

Transformation of DNA into a Bacillus licheniformis strain as described in U.S. Pat. No. 5,352,604 is performed via electroporation. Preparation of electrocompetent Bacillus licheniformis cells and transformation of DNA is performed as essentially described by Brigidi et al (Brigidi, P., Mateuzzi, D. (1991). Biotechnol. Techniques 5, 5) with the following modification: Upon transformation of DNA, cells are recovered in 1 ml LBSPG buffer and incubated for 60 min at 37° C. (Vehmaanpers J., 1989, FEMS Microbio. Lett., 61: 165-170) following plating on selective LB-agar plates.

In order to overcome the Bacillus licheniformis specific restriction modification system of Bacillus licheniformis strains, plasmid DNA is isolated from Ec #098 cells as described below.

### Plasmid Isolation

Plasmid DNA was isolated from Bacillus and E. coli cells by standard molecular biology methods described in (Sambrook, J. and Russell, D. W. Molecular cloning. A laboratory manual, 3rd ed, Cold Spring Harbor Laboratory Press, Cold Spring Harbor, N Y. 2001) or the alkaline lysis method (Birnboim, H. C., Doly, J. (1979). Nucleic Acids Res 7(6): 1513-1523). Bacillus cells were in comparison to E. coli treated with 10 mg/ml lysozyme for 30 min at 37 C prior to cell lysis.

### Molecular Biology Methods and Techniques

Standard methods in molecular biology not limited to cultivation of Bacillus and E. coli microorganisms, electroporation of DNA, isolation of genomic and plasmid DNA, PCR reactions, cloning technologies were performed as essentially described by Sambrook and Russell. (Sambrook, J. and Russell, D. W. Molecular cloning. A laboratory manual, 3rd ed, Cold Spring Harbor Laboratory Press, Cold Spring Harbor, N Y. 2001.)

### Strains

E. coli Strain Ec #098

E. coli strain Ec #098 is an E. coli INV110 strain (Life technologies) carrying the DNA-methyltransferase encoding expression plasmid pMDS003 WO2019016051.

Generation of Bacillus licheniformis Gene k.o Strains

For gene deletion in a Bacillus licheniformis strain as described in U.S. Pat. No. 5,352,604 and derivatives thereof deletion plasmids were transformed into E. coli strain Ec #098 made competent according to the method of Chung (Chung, C. T., Niemela, S. L., and Miller, R. H. (1989). One-step preparation of competent Escherichia coli: transformation and storage of bacterial cells in the same solution. Proc. Natl. Acad. Sci. U.S.A 86, 2172-2175), following selection on LB-agar plates containing 100 μg/ml ampicillin and 30 μg/ml chloramphenicol at 37° C. Plasmid DNA was isolated from individual clones and analyzed for correctness by PCR analysis. The isolated plasmid DNA carries the DNA methylation pattern of Bacillus licheniformis as described in WO2019016051 and is protected from degradation upon transfer into Bacillus licheniformis.

aprE Gene Deletion Strain Bli #002

Electrocompetent Bacillus licheniformis cells as described in U.S. Pat. No. 5,352,604 were prepared as described above and transformed with 1 μg of pDe1003 aprE gene deletion plasmid isolated from E. coli Ec #098 following plating on LB-agar plates containing 5 μg/ml erythromycin at 30° C.

The gene deletion procedure was performed as described in the following: Plasmid carrying Bacillus licheniformis cells were grown on LB-agar plates with 5 μg/ml erythromycin at 45° C. forcing integration of the deletion plasmid via Campbell recombination into the chromosome with one of the homology regions of pDe1003 homologous to the sequences 5′ or 3′ of the aprE gene. Clones were picked and cultivated in LB-media without selection pressure at 45° C. for 6 hours, following plating on LB-agar plates with 5 μg/ml erythromycin at 30° C. Individual clones were picked and analyzed by colony-PCR with oligonucleotides SEQ ID 066 and SEQ ID 067 for successful deletion of the aprE gene. Putative deletion positive individual clones were picked and taken through two consecutive overnight incubation in LB media without antibiotics at 45° C. to cure the plasmid and plated on LB-agar plates for overnight incubation at 30° C. Single clones were again restreaked on LB-agar plates with 5 μg/ml erythromycin and analyzed by colony PCR for successful deletion of the aprE gene. A single erythromycin-sensitive clone with the correct deleted aprE gene was isolated and designated Bli #002

amyB Gene Deletion Strain Bli #003

Electrocompetent Bacillus licheniformis Bli #002 cells were prepared as described above and transformed with 1 μg of pDe1004 amyB gene deletion plasmid isolated from E. coli Ec #098 following plating on LB-agar plates containing 5 μg/ml erythromycin at 30° C.

The gene deletion procedure was performed as described for the aprE gene.

The deletion of the amyB gene was analyzed by PCR with oligonucleotides SEQ ID 069 and SEQ ID 070. The resulting Bacillus licheniformis strain with a deleted aprE and deleted amyB gene is designated Bli #003.

sigF Gene Deletion Strain Bli #004

Electrocompetent Bacillus licheniformis Bli #003 cells were prepared as described above and transformed with 1 μg of pDel005 sigF gene deletion plasmid isolated from E. coli Ec #098 following plating on LB-agar plates containing 5 μg/ml erythromycin at 30° C.

The gene deletion procedure was performed as described for the aprE gene.

The deletion of the sigF gene was analyzed by PCR with oligonucleotides SEQ ID 071 and SEQ ID 072 The resulting Bacillus licheniformis strain with a deleted aprE, a deleted amyB gene and a deleted sigF gene is designated Bli #004. Bacillus licheniformis strain Bli #004 is no longer able to sporulate as described (Fleming et al., Appl Environ Microbiol. 1995 November; 61(11):3775-80).

### Poly-Gamma Glutamate Synthesis Genes Deletion Strain Bli #008

Electrocompetent Bacillus licheniformis Bli #004 cells were prepared as described above and transformed with 1 μg of pDel007 pga gene deletion plasmid isolated from E. coli Ec #098 following plating on LB-agar plates containing 5 μg/ml erythromycin at 30° C.

The gene deletion procedure was performed as described for the deletion of the aprE gene.

The deletion of the pga genes was analyzed by PCR with oligonucleotides SEQ ID 075 and SEQ ID 076 The resulting Bacillus licheniformis strain with a deleted aprE, a deleted amyB gene, a deleted sigF gene and a deleted pga gene cluster is designated Bli #008.

### Plasmids

pEC194RS—Bacillus Temperature Sensitive Deletion Plasmid.

The plasmid pE194 (Villafane, et al (1987): J. Bacteriol. 169(10), 4822-4829) is PCR-amplified with oligonucleotides SEQ ID 061 and SEQ ID 062 with flanking PvuII sites, digested with restriction endonuclease PvuII and ligated into vector pCE1 digested with restriction enzyme SmaI. pCE1 is a pUC18 derivative, where the BsaI site within the ampicillin resistance gene has been removed by a silent mutation. The ligation mixture was transformed into E. coli DH10B cells (Life technologies). Transformants were spread and incubated overnight at 37 C on LB-agar plates containing 100 μg/ml ampicillin. Plasmid DNA was isolated from individual clones and analyzed for correctness by restriction digest. The resulting plasmid is named pEC194S.

The type-Il-assembly mRFP cassette is PCR-amplified from plasmid pBSd141R (accession number: KY995200) (Radeck, J., Meyer, D., Lautenschlager, N., and Mascher, T. 2017. Bacillus SEVA siblings: A Golden Gate-based toolbox to create personalized integrative vectors for Bacillus subtilis. Sci. Rep. 7:14134) with oligonucleotides SEQ ID 063 and SEQ ID 064, comprising additional nucleotides for the restriction site BamHI. The PCR fragment and pEC194S were restricted with restriction enzyme BamHI following ligation and transformation into E. coli DH10B cells (Life technologies). Transformants were spread and incubated overnight at 37 C on LB-agar plates containing 100 μg/ml ampicillin. Plasmid DNA was isolated from individual clones and analyzed for correctness by restriction digest. The resulting plasmid pEC194RS carries the mRFP cassette with the open reading frame opposite to the reading frame of the erythromycin resistance gene.

pDeI003—aprE Gene Deletion Plasmid

The gene deletion plasmid for the aprE gene of Bacillus licheniformis was constructed with plasmid pEC194RS and the gene synthesis construct SEQ ID 065 comprising the genomic regions 5′ and 3′ of the aprE gene flanked by BsaI sites compatible to pEC194RS. The type-Il-assembly with restriction endonuclease BsaI was performed as described (Radeck et al., 2017) and the reaction mixture subsequently transformed into E. coli DH10B cells (Life technologies). Transformants were spread and incubated overnight at 37 C on LB-agar plates containing 100 μg/ml ampicillin. Plasmid DNA was isolated from individual clones and analyzed for correctness by restriction digest. The resulting aprE deletion plasmid is named pDel003.

pDeI004—amyB Gene Deletion Plasmid

The gene deletion plasmid for the amyB gene of Bacillus licheniformis was constructed as described for pDel003, however the gene synthesis construct SEQ ID 068 comprising the genomic regions 5′ and 3′ of the amyB gene flanked by BsaI sites compatible to pEC194RS was used. The resulting amyB deletion plasmid is named pDel004.

pDel005—sigF Gene Deletion Plasmid

The gene deletion plasmid for the sigF gene (spoIIAC gene) of Bacillus licheniformis was constructed as described for pDel003, however the gene synthesis construct SEQ ID 070 comprising the genomic regions 5′ and 3′ of the sigF gene flanked by BsaI sites compatible to pEC194RS was used. The resulting sigF deletion plasmid is named pDel005.

pDel007—Poly-Gamma-Glutamate Synthesis Genes Deletion Plasmid

The deletion plasmid for deletion of the genes involved in poly-gamma-glutamate (pga) production, namely ywsC (pgsB), ywtA (pgsC), ywtB (pgsA), ywtC (pgsE) of Bacillus licheniformis was constructed as described for pDel003, however the gene synthesis construct SEQ ID 074 comprising the genomic regions 5′ and 3′ flanking the ywsC, ywtA (pgsC), ywtB (pgsA), ywtC (pgsE) genes flanked by BsaI sites compatible to pEC194RS was used. The resulting pga deletion plasmid is named pDel007.

### Example 1: Sequence Based Identification of Genes Belonging to Enzymes with Putative Lipase/Esterase Activity

Putative lipases/esterases were identified using hidden markov models (HMMs) from the Pfam database (version 33.1, https:dpfam.xfam.org/; Sara El-Gebali, Robert D Finn; The Pfam protein families database in 2019, Nucleic Acids Research, Volume 47, Issue D1, 8 Jan. 2019, Pages 0427-0432). The following HMMs were searched in the proteome of Bacillus licheniformis DSM13: PF00657.23, PF01674.19, PF01764.26, PF02230.17, PF03280.15, PF03583.15 PF13472.7, PF14606.7 using HMMER 3.1 (http://hmmer.org/) (J. Mistry, M. Punta; Challenges in Homology Search: HMMER3 and Convergent Evolution of Coiled-Coil Regions, Nucleic Acids Research, 41:el21, 2013). Specifically, hmmsearch was run with default parameters and all resulting hits considered.

The identified proteins and their corresponding genes with gene name annotation and function are listed in Table 1.

TABLE 1

| Gene | | | | |
|---|---|---|---|---|
| name | Function | PRT | DNA | Accession |
| yhfR | putative phosphatase | SEQ ID 1 | SEQ ID 31 | PF14606.7 |
| yitV | putative peptidase | SEQ ID 2 | SEQ ID 32 | PF02230.17 |
| yesT | rhamnogalacturonan acetylesterase | SEQ ID 3 | SEQ ID 33 | PF13472.7 |
| yesT | rhamnogalacturonan acetylesterase | SEQ ID 3 | SEQ ID 33 | PF00657.23 |
| unknown | putative carbohydrate esterase | SEQ ID 4 | SEQ ID 34 | PF13472.7 |
| unknown | putative carbohydrate esterase | SEQ ID 4 | SEQ ID 34 | PF00657.23 |
| yesY | putative rhamnogalacturonan | SEQ ID 5 | SEQ ID 35 | PF13472.7 |
| | acetylesterase | | | |
| yesY | putative rhamnogalacturonan | SEQ ID 5 | SEQ ID 35 | PF00657.23 |
| | acetylesterase | | | |
| unknown | hypothetical protein | SEQ ID 6 | SEQ ID 36 | PF01764.26 |
| fliG | flagellar motor switch protein | SEQ ID 7 | SEQ ID 37 | PF03280.15 |
| yisY | AB hydrolase superfamily protein | SEQ ID 8 | SEQ ID 38 | PF01764.26 |
| yisY | AB hydrolase superfamily protein | SEQ ID 8 | SEQ ID 38 | PF03583.15 |
| yisY | AB hydrolase superfamily protein | SEQ ID 8 | SEQ ID 38 | PF02230.17 |
| mhqD | putative hydrolase | SEQ ID 9 | SEQ ID 39 | PF02230.17 |
| ypmR | putative esterase | SEQ ID 10 | SEQ ID 40 | PF13472.7 |
| ypmR | putative esterase | SEQ ID 10 | SEQ ID 40 | PF00657.23 |
| ypmR | putative esterase | SEQ ID 10 | SEQ ID 40 | PF14606.7 |
| lip | putative lipase | SEQ ID 11 | SEQ ID 41 | PF01674.19 |
| yqiL | hypothetical protein | SEQ ID 12 | SEQ ID 42 | PF01764.26 |
| yqeF | putative lipoprotein | SEQ ID 13 | SEQ ID 43 | PF13472.7 |
| yqeF | putative lipoprotein | SEQ ID 13 | SEQ ID 43 | PF00657.23 |
| unknown | putative lipolytic enzyme | SEQ ID 14 | SEQ ID 44 | PF01764.26 |
| unknown | putative lipolytic enzyme | SEQ ID 14 | SEQ ID 44 | PF02230.17 |
| ytaP | putative hydrolase | SEQ ID 15 | SEQ ID 45 | PF03583.15 |
| ytpA | phospholipase | SEQ ID 16 | SEQ ID 46 | PF02230.17 |
| ytmA | putative dipeptidylpeptidase | SEQ ID 17 | SEQ ID 47 | PF02230.17 |
| ytmA | putative dipeptidylpeptidase | SEQ ID 17 | SEQ ID 47 | PF03583.15 |
| ytxM | 2-succinyl-6-hydroxy-2, 4-cyclo- | SEQ ID 18 | SEQ ID 48 | PF02230.17 |
| | hexadiene-1-carboxylate synthase | | | |
| yugF | alpha/beta hydrolase | SEQ ID 19 | SEQ ID 49 | PF02230.17 |
| yugF | alpha/beta hydrolase | SEQ ID 19 | SEQ ID 49 | PF01764.26 |
| unknown | dipeptidylpeptidase | SEQ ID 20 | SEQ ID 50 | PF03583.15 |
| unknown | dipeptidylpeptidase | SEQ ID 20 | SEQ ID 50 | PF02230.17 |
| yuxL | putative acylaminoacyl-peptidase | SEQ ID 21 | SEQ ID 51 | PF02230.17 |
| unknown | phage putative hydrolase protein | SEQ ID 22 | SEQ ID 52 | PF01764.26 |
| besA | trilactone hydrolase | SEQ ID 23 | SEQ ID 53 | PF02230.17 |
| unknown | rhamnogalacturonan acetylesterase | SEQ ID 24 | SEQ ID 54 | PF13472.7 |
| IchAD | lichenysin synthase | SEQ ID 25 | SEQ ID 55 | PF02230.17 |
| unknown | putative esterase | SEQ ID 26 | SEQ ID 56 | PF02230.17 |
| lipC | spore germination lipase LipC | SEQ ID 27 | SEQ ID 57 | PF13472.7 |
| lipC | spore germination lipase LipC | SEQ ID 27 | SEQ ID 57 | PF00657.23 |
| unknown | putative esterase/lipase | SEQ ID 28 | SEQ ID 58 | PF02230.17 |
| unknown | putative esterase/lipase | SEQ ID 28 | SEQ ID 58 | PF03583.15 |
| yrhL | putative peptidoglycan O-acetyltransferase | SEQ ID 29 | SEQ ID 59 | PF13472.7 |
| yrhL | putative peptidoglycan O-acetyltransferase | SEQ ID 29 | SEQ ID 59 | PF00657.23 |
| unknown | hypothetical protein | SEQ ID 30 | SEQ ID 60 | PF02230.17 |

### Example 2: Identification of Lipase/Esterase Activity by Fractionation of Bacillus Licheniformis Fermentation Supernatant

In order to characterize the esterase or lipase activity present in Bacillus licheniformis cultivations a Bacillus licheniformis (DaprE DamyB DsigF Dpga) was cultivated in a fed-batch fermentation as described in WO2020169563A1. The biomass was separated using a dead-end filtration and pretreatment of the fermentation broth with flocculant. The fermentation broth was pretreated using pDADMAC flocculation solution before the filtration step: 40 g Magnafloc® LT7996 (˜20% w/v pDADMAC content) per 1 L of fermentation broth was diluted to the respective volume using deionized water to make up the flocculation solution. This solution was subsequently mixed 1:1 with the fermentation broth under constant stirring. After 15 min the pretreated broth was subjected to a three-step dead-end filtration: In a first step the biomass was separated using a dead-end filtration pressure nutsche unit on 200 mL scale using a high porosity perlite filter aid and a diatomite precoat: Body feed: 40 g/kg Harbolite 900; Precoat: 0.05 g/cm2 Dicalite BF. Subsequent filtration steps using depth filters K250 and EKS (Pall) yielded clear supernatant for subsequent chromatographic purification to isolate the lipolytic activity.

For anion exchange chromatography 10 mL sterile filtrated material was adjusted to 5 mS using 20 mM MOPS pH7.0 and loaded on a 6 mL Resource Q column (GE Healthcare). The column was washed with buffer A (20 mM MOPS, pH 7 supplemented with 5 mM MgCl2) until the baseline was achieved. Elution of bound material was performed using a linear gradient to buffer B (20 mM MOPS, pH 7 supplemented with 1 M NaCl and 5 mM MgCl2) over 40 column volumes with fractions being collected overtime. Esterase/Lipase activity was determined using pNP-butyrate as substrate. Herefore, the different fractions were mixed in a 1:1 ratio with 2 mM pNP-butyrate (in 10% DMSO, 100 mM MOPS buffer pH7, 100 mM NaCl, 0.045% Brij L23). The esterase/lipase activity was measured by following the absorbance increase over time at 405 nm. Using the activity data the lipase/esterase containing fractions identified and are used for LC-MS/MS analysis. The derived sample is called S1_IEX.

Orthogonally a hydrophobic interaction chromatography was performed using a HiPrep Butyl FF column (GE Healthcare). For chromatography 30 mL sterile filtrated material was adjusted to 25% saturation of ammonium sulfate and pH 7.0 using 20 mM MOPS. The material was loaded on a 20 mL HiPrep Butyl FF column (GE Healthcare). After loading the column, buffer A (20 mM MOPS, pH 7 supplemented with 5 mM MgCl2 and 25% saturation of Ammonium sulfate) was loaded until the baseline was achieved. Elution of bound material was performed using a linear gradient to buffer B (20 mM MOPS, pH 7 supplemented with 5 mM MgCl2) over 20 column volumes with fractions being collected over time. Esterase/Lipase activity was determined using pNP-butyrate as substrate. Herefore, the different fractions were mixed in a 1:1 ratio with 2 mM pNP-butyrate (in 10% DMSO, 100 mM MOPS buffer pH7, 100 mM NaCl, 0.045% Brij L23). The esterase/lipase activity was measured by following the absorbance increase over time at 405 nm. Using the activity data the lipase/esterase containing fractions are identified and are used for LCMS/MS analysis. The derived sample is called S2_HIC.

For LC-MS/MS the second sample set was processed with a S-Trap micro approach to desalt and digest the samples with trypsin. The generated peptides were analyzed via nanoLC-HRMS/MS. The acquired data was searched with the software MaxQuant against the Bacillus licheniformis proteome of DSM13 (strain ATCC 14580/DSM 13/JCM 2505/NBRC 12200/NCIMB 9375/NRRL NRS-1264/Gibson 46) with carbamidomethylation of cysteine as fixed and methionine oxidation and acetylated N-Term as variable modifications.

For the identification of the proteins only unique peptides were used and to reduce the number of false positive assigned spectra with an FDR cut-off of 0.01 was used. The protein list for the S1_IEX and S2_HIC samples was filtered for proteins that were identified by more than one peptide.

The protein list for the each of the samples was filtered for proteins that were identified by more than two peptides and an andromeda score better than 10. As evaluation the tables resulting from LC-MS/MS were compared to the table derived from example 1 in order to identify proteins being present in both analysis. In table 2 two proteins from the putative lipases/esterases (see Table 1) are depicted present in the fractions enriched for lipolytic activity using both purifications (ion exchange chromatography derived sample S1_IEX and hydrophobic interaction chromatography S2_HIC).

TABLE 2

| | | Sequence | Mol. weight | | |
|---|---|---|---|---|---|
| Name | Peptides | coverage [%] | [kDa] | gene | SEQ ID |
| YitV | 23 | 66,3 | 29,778 | yitV | 2 |
| YpmR | 6 | 29,6 | 28,469 | ypmR | 10 |

### Example 3: Gene Knockout of Genes Encoding for Lipases/Esterases in Bacillus licheniformis

The genes yitV (SEQ ID 032) and ypmR (SEQ ID040) were deleted the in Bacillus licheniformis strain Bli #008.

Therefore, the yitV gene deletion plasmid was constructed as described above by cloning of the 5′ and 3′ flanking homologous regions into the Bacillus/E. coli shuttle plasmid pEC194RS. The 5′ homologous region and the 3′ homologous region were PCR-amplified with oligonucleotides SEQ ID 077 and SEQ ID 078, and oligonucleotides SEQ ID 079 and SEQ ID 080 respectively. The outermost oligonucleotides were flanked by BsaI sites compatible to pEC194RS. The type-Il-assembly with restriction endonuclease BsaI was performed as described (Radeck et al., 2017; Sci. Rep. 7: 14134) and the reaction mixture was subsequently transformed into E. coli DH10B cells (Life technologies). Transformants were spread and incubated overnight at 37 C on LB-agar plates containing 100 μg/ml ampicillin. Plasmid DNA was isolated from individual clones and was analyzed for correctness by restriction digest. The resulting yitV gene deletion plasmid is named pDe1043 and was used for subsequent deletion in Bacillus licheniformis Bli #008.

The ypmR gene deletion plasmid was constructed as described for the yitV gene deletion plasmid, but the 5′ homologous region and the 3′ homologous region were PCR-amplified with oligonucleotides SEQ ID 081 and SEQ ID 082, and oligonucleotides SEQ ID 083 and SEQ ID 084 respectively.

The resulting ypmR gene deletion plasmid was used for subsequent deletion in Bacillus licheniformis Bli #008.

yitV Gene Deletion Strain Bli #080

Electrocompetent Bacillus licheniformis Bli #008 cells were prepared as described above and transformed with 1 μg of pDe1043 yitV gene deletion plasmid isolated from E. coli Ec #098 followed by plating on LB-agar plates containing 5 μg/ml erythromycin and incubation at 30° C. The gene deletion procedure was performed as described for the aprE gene and successful gene deletion was monitored by colony-PCR.

ypmR Gene Deletion Strain Bli #081

Electrocompetent Bacillus licheniformis Bli #008 cells were prepared as described above and transformed with 1 μg of pDe1044 ypmR gene deletion plasmid isolated from E. coli Ec #098 followed by plating on LB-agar plates containing 5 μg/ml erythromycin and incubation at 30° C.

The gene deletion procedure was performed as described for the aprE gene and successful gene deletion was monitored by colony-PCR.

The combinatorial knockout strain comprising inactivated genes yitV (SEQ ID 032) and ypmR (SEQ ID 040) was constructed as described in the following.

yitV and ypmR Gene Deletion Strain Bli #082

Electrocompetent Bacillus licheniformis Bli #081 cells with deleted yitV gene were prepared as described above and transformed with 1 μg of pDe1044 ypmR gene deletion plasmid isolated from E. coli Ec #098 followed by plating on LB-agar plates containing 5 μg/ml erythromycin and incubation at 30° C.

The gene deletion procedure was performed as described for the aprE gene and successful gene deletion was monitored by colony-PCR.

### Example 4 Lipase Activity of Strains Having the Lipases Deleted

In order to characterize the strains according the invention cultivations of Bacillus licheniformis strains Bli #008(DaprE DamyB DsigF Dpga), Bli #080 (DaprE DamyB DsigF Dpga, DyitV), Bli #081 (DaprE DamyB DsigF Dpga, DypmR), and Bli #082 (DaprE DamyB DsigF Dpga, DyitV, DypmR) were tested and compared to the Bacillus licheniformis strain Bli #008 lacking the deletion(s) of yitV and/or ypmR. Herefore, the strains were cultivated as described in Example 2. After harvesting the fermenter, the broth was treated and chromatographic fractionation was performed as described in Example 2.

The esterase/lipase activity in the YitV and YpmR containing fractions of the reference strain Bli #008 and the corresponding fractions of the deletion strains Bli #080, Bli #081, and Bli #082 were determined using pNP-butyrate as substrate. Herefore, the different samples were mixed in a 1:1 ratio with 2 mM pNP-butyrate (in 10% DMSO, 100 mM MOPS buffer pH7, 100 mM NaCl, 0.045% Brij L23). The esterase/lipase activity was measured by following the absorbance increase over time at 405 nm. See below the activity for the respective strains (one unit is defined as the release of 1 μmol pNP in 1 min:

| | | Lipase activity rel. |
|---|---|---|
| Strain | Genotype | to control strain |
| Bli#008 (control strain) | DaprE DamyB DsigF Dpga | 100% |
| Bli#082 | DaprE DamyB DsigF Dpga | <4% |
| | DyitV DypmR | |
| Bli#080 | DaprE DamyB DsigF Dpga | 22% |
| | DyitV | |
| Bli#081 | DaprE DamyB DsigF Dpga | 36% |
| | DypmR | |

It can be seen that the deletion of YitV and/or YpmR reduces the lipolytic activity compared to the wildtype.

1. A recombinant Bacillus cell comprising an inactivation of the yitV and ypmR gene.

2. The recombinant Bacillus cell of claim 1, wherein the Bacillus cell produces a protein of interest.

3. The recombinant Bacillus cell of claim 1, comprising a gene encoding for the protein of interest.

4. The recombinant Bacillus of claim 1, wherein the Bacillus cell is selected from the group including consisting of Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus methylotrophicus, Bacillus cereus Bacillus paralicheniformis, Bacillus subtilis, and Bacillus thuringiensis cells.

5. The recombinant Bacillus cell of claim 1, wherein the yitV gene encodes a YitV protein that is selected from the group consisting of:
(a) a YitV protein having at least 70% identity with SEQ ID NO: 2;
(b) a YitV protein encoded by a polynucleotide having at least 70% identity with SEQ ID NO: 32;
(c) a YitV protein encoded by a polynucleotide that hybridizes under high stringency conditions with (i) a polynucleotide comprising SEQ ID NO: 32 or (ii) the full-length complement of (i);
(d) a variant of the YitV protein of SEQ ID NO: 2 comprising a substitution, deletion, and/or insertion at one or more positions and having lipase and/or esterase activity;
(e) a YitV protein encoded by a polynucleotide that differs from SEQ ID NO: 32 due to the degeneracy of the genetic code.

6. The recombinant Bacillus cell of claim 1, wherein the ypmR gene encodes a YpmR protein that is selected from the group consisting of:
(a) a YpmR protein having at least 70% identity with SEQ ID NO: 10;
(b) a YpmR protein encoded by a polynucleotide having at least 70% identity with SEQ ID NO: 40;
(c) a YpmR protein encoded by a polynucleotide that hybridizes under high stringency conditions with (i) a polynucleotide comprising SEQ ID NO: 40 or (ii) the full-length complement of (i);
(d) a variant of the YpmR protein of SEQ ID NO: 10 comprising a substitution, deletion, and/or insertion at one or more positions and having lipase and/or esterase activity;
(e) a YpmR protein encoded by a polynucleotide that differs from SEQ ID NO: 40 due to the degeneracy of the genetic code.

7. The recombinant Bacillus cell of claim 2, wherein the protein of interest is an enzyme.

8. A method for producing a compound of interest, comprising the steps of
a) providing a modified Bacillus host cell comprising an inactivation of the yitV and ypmR gene,
b) introducing into said modified Bacillus host cell an expression cassette for a compound of interest,
c) cultivating the host cell under conditions which allow for the expression of the compound of interest, and
d) optionally isolating the compound of interest from the cultivation medium.

9. A composition comprising a compound of interest with reduced contamination with lipase and/or esterase activity obtained by the method of claim 8.

10. Food or feed or detergent composition, comprising the composition of claim 9.

11. A method for reducing the lipase and/or esterase activity in a Bacillus cell comprising inactivating the yitV and/or ypmR gene in the Bacillus cell.

12. A method for reducing the lipase and/or esterase activity of a compound of interest produced by a Bacillus cell comprising the steps of
a) providing a modified Bacillus host cell comprising an inactivation of the yitV and/or ypmR gene,
b) introducing into said modified Bacillus host cell an expression cassette for a compound of interest,
c) cultivating the host cell under conditions which allow for the expression of the compound of interest, and
d) optionally isolating the compound of interest from the cultivation medium.

13. A method for reducing the formation of malodour or off-flavor in a composition comprising a compound of interest produced by a Bacillus cell comprising the steps of
a) providing a modified Bacillus host cell comprising an inactivation of the yitV and/or ypmR gene,
b) introducing into said modified Bacillus host cell an expression cassette for a compound of interest,
c) cultivating the host cell under conditions which allow for the expression of the compound of interest, and
d) optionally isolating the compound of interest from the cultivation medium.

14. The recombinant Bacillus cell of claim 7, wherein the protein of interest is an enzyme selected from the group consisting of amylase, alpha-amylase, glucoamylase, pullulanase, protease, lipase, cutinase, acyl transferase, cellulase, endoglucanase, glucosidase, cellubiohydrolase, lactase, xylanase, xyloglucantransferase, xylosidase, mannanase, phytase, phosphatase, xylose isomerase, glucoase isomerase, acetolactate decarboxylase, pectinase, pectin methylesterase, polygalacturonidase, lyase, pectate lyase, arabinase, arabinofuranosidase, galactanase, laccase, peroxidase and an asparaginase


SEQUENCE LISTING (84 sequences)

SEQ ID NO: 1 - protein, 190 aa, Bacillus licheniformis
  putative phosphatase (yhfR)
MTAICLIRHGETDWNALGKLQGRTDIPLNETGKKQAKETGAFLKGSDWDVIITSPLRRAKETAEIINQYLGLEIIEMEDFIERNYGDAEGMPFEERMRLYPDKEYPNQESKEALAERLMAGVQKVSERYPDKKVLIVAHGAAIHALLSKISNGDINLENTKLVNACLSNIKFHENKWHVKDYNLSHHLSD

SEQ ID NO: 2 - protein, 255 aa, Bacillus licheniformis
  putative peptidase (yitV)
MIIIEKQHISNIPLLHIVKEEKKEKALPLVFFIHGFTSAKEHNLHFAYLLAEKGMRVILPEALYHGERNEQLTQEELAPRFWEIVTNEIKELEVLKNHFERENMIEKGRIGAAGTSMGGIVTLGALTQYEWITTAVSLMGSPAYVEFFDQQLAFMREKKIELPITEEQIDQQREELKRFDLSLQPDKLNMRPLLFWHGKQDGTVPFALTRRFYESIIPLYEARPDLLHFIEDERAGHKVSREGLLKTVEWFDAHL

SEQ ID NO: 3 - protein, 238 aa, Bacillus licheniformis
  rhamnogalacturonan acetylesterase (yesT)
MKRTDKKPKVTVYLAGDSTVADCPPHEAPMAGWGQMLPLFFSDEAAVVNMAKGGASSNSFIDEGRLDDITERLAPGDYVLIQFGHNDQKPYGTDPYTTYQEHLVRYVEAVREKQATPVLITSAERRRFDQNGKPADTLGDFPKAMKALADSFDVPLIDLWSKTKALYESLGIEGSKRLFVHFQPHEHPHYPNGIEDNTHFSEAGARQVARLVTEGIRELGLPLSAYLLCERSDFRAGV

SEQ ID NO: 4 - protein, 421 aa, Bacillus licheniformis
  putative carbohydrate esterase (unknown)
MPKEKKWGRSRPEQREAQPKKRSPFRWKTGASIWSEGASAKGVCHRNFEVYQFDFGDGPLASGFIPVGAASAYTPLKGYGFDDAAKVSCTDRGTADPLRSDFCIPDGTAFTVDLPDGQYQVQIITGDARAKTNMDVRANGMLQLYSFGAEAGGYNEKTFPIHISGGRLSIQALTARTMPRLNALVITKTAYHVTDDNTTVFLAGDSTVSDYEPGYAPMAGWGQFLGGYFSDEIVIDNQAKRARSAKSFVDERSLATILNRMKRGDYLLIQFGINDAGQDPARHTDPYTTFQEYLRTYINKAREKGGRHVLVTSQSKRTYDEKGVFYNSIGEYPNAMRQLGREMNVPVIDLNKKSIEYYNEIGVEATKNVFMFLEPGESPNYPEGIQDRVHFHEFGANQLARLVTGEISDLNIYPLKHSLKK

SEQ ID NO: 5 - protein, 214 aa, Bacillus licheniformis
  putative rhamnogalacturonan acetylesterase (yesY)
MTISIYLAGDSTVQHYKADSPQGGWGEFLQSYLSGNVQVINRAIGGRSSKTFVEEGRLASILDVIKQDDWLFVQMGHNDASKDKPERYTEPYTTYKQYLKMYVDGARQKGASPLLITPVARLHYKDGVFLNDFPDYCIAMKQVASEENVRLIDLMDKSLQHFAEKGYSEVFKYFMISENVNDYTHFTKKGAREMAKLVSEGIRELGLPFVKETV

SEQ ID NO: 6 - protein, 371 aa, Bacillus licheniformis
  hypothetical protein (unknown)
MYLNKKSDIPKLTDKEYYFLSQNTYSTDKMKEAFKERTPIESKSNKAFFVDKIKRDSDTGLDAYVFVQAKKKDGKWVKPNAPENVVVAFAGTNPKEQFFQDVIDADGGNVVMGLDPKKKSQYIIEKDAKDTSKTIGKYNATPSQDAMLSTGKYKLITKTSQIGQADDLVREVKQKYKGTSTVISTTGHSLGGAEAEYSAVNNDIYAVAFNNPSVVKLHSEEKQKEIRSGKYDSSVKAIVNPDDMTGSGWWNEYERHAGRTIYTKDPSTSRVERQIRLDPKYSGGIFGTVFNVAVDYIATTAMGMPDTHGLNKGNFTFDENGNVQNIEGDELVYDKNLKAMLPPEVASGSGAIKVTPEVAKQLAQKVNACGR

SEQ ID NO: 7 - protein, 338 aa, Bacillus licheniformis
  flagellar motor switch protein (fliG)
MAKMKQDRLTGKQKAAILMISLGLDVSASVYKHLTDEEIERLTLEISNVRAVSPEKKDEIIQEFHETAIAQEYISQGGIHYAKQVLDKALGEDKAASIINRLTSSLQVRPFDFARKADPEQILNFIQHEHPQTIALILSYLDPVQSGQILSELNQDVQAEVARRIAVMDRTSPEIINEVEQILEQKLSSTFTQDYTQTGGIEAVVEVLNGVDRGTEKTILDALEIQDPELADEIKKRMFVFEDIVTLDNRAIQRVIRDVENDDLLLSLKVASEEVKDIVFSNMSQRMAETFKEEMEFMGPVRLRDVEEAQSRIVSVIRRLEEAGEIVIARGGGDDIIV

SEQ ID NO: 8 - protein, 286 aa, Bacillus licheniformis
  AB hydrolase superfamily protein (yisY)
MFSPRRHSPIYILGVSYGKDMIGGDGMGYFIEAEPSVNLYVEDINPQSGKAIVFLHGWPLSHLQFEYQFDDLSRKGYRCIGIDWRGFGRSDKPATGYNYNRLADDIRAVVEALQLNNFTLAGHSTGGAIALRYMSRYRGAGVSKLVLIDAAAPVGFTEETAAKLLQQASNDRPKMMREVTDTFFFQYITQPFSDCFFQLGLQAAGWSTKAVIEMLRDEKLYDDPEKVAAPALIIHGIQDKVIPFAQAKELSRKIRNSYLVPFQYSGHGPFWEERKMFNRILTQFIG

SEQ ID NO: 9 - protein, 201 aa, Bacillus licheniformis
  putative hydrolase (mhqD)
MNYIYEKGDSDLTLLLLHGTGGNEHDLLPIGKMIDPDANLLGVRGNVLENGMPRFFRRLREGVFDEKDLIERTDELKTFLDEAAKELHFDRSRVVAIGYSNGANIAGSLLFRHEHVLKGAILHHPMVPFRNRKLPDMTGLPVFIGAGKTDPLCPPQESSELAGYFTNAGAKVLLHWEGGGHQLTHGEIEKAKIWYEKHIKA

SEQ ID NO: 10 - protein, 250 aa, Bacillus licheniformis
  putative esterase (ypmR)
MNIRFITVMMALVCLLSACTEWNAGVEKTSVSPKRDIVIAAVGDSLTEGVGDQEKKGYVGMVADELESRSDVKSVTVKNYAVKGSRTDQLLERLKDKEVQEGLKDADYILFTIGGNDLMKVVRQNFAHLTLTPFRAEQKLFEKRFSNILAEIREQNASAELIYVSMYNPFKFTLSELREVDQVVDEWNEGAEKRLKKVSNTKMADIADIFEEYSDEKKIAEDEFHPNQYGYSLIAKRVYEQIKNEDLPAE

SEQ ID NO: 11 - protein, 204 aa, Bacillus licheniformis
  putative lipase (lip)
MRRHSFLSILLICMLSVVSVFSFRPSAASAASHNPVVMVHGIGGADYNFIGIKSYLQSQGWTSSELYAINFIDKTGNNINNAPRLSEYIKRVLNQTGASKVDIVAHSMGGANTLYYIKNLDGADKVGHVVTLGGANRLVTNTAPQNDKISYTSIYSTSDYIVLNSLSKLDGANNVQISGVSHVGLLFSSKVNALIKDGLTASGK

SEQ ID NO: 12 - protein, 253 aa, Bacillus licheniformis
  hypothetical protein (yqiL)
MTITERIYTVEGCMFYTKQRKGKKNATIIFEAGYGFSSTTWQPILSDIDEDLGLFAYDRAGLGKSGPSTSKRTADQMVKELKALLAAADVKPPYLFVAHSFGAVIARLFASRYSDEVIGMVLLDPACEDQEEKVLPLLPDTLKEDYVKQFSIEGSHEDFKQSLDMLKQEKKHLGSVPLLVLSAGKRTEAAKQAHQEWLKLHSRLLSLSHQSGWIQAKNSSHLIHHDEPHIVQLAIYDVWYAAQQQTYAYQAAN

SEQ ID NO: 13 - protein, 243 aa, Bacillus licheniformis
  putative lipoprotein (yqeF)
MKKSLVMVLVFLLLLSGCGEDDPRKVVAFGDSNTRGSNWQFRDYPKAEKWVNLLQTSLQGQYKINNAGIGGETTEDARFRFRKDVLDKQPAYLFIMFGTNDAAILTKGLPRVSKKRFKENLYYFVKESRRRGIEPVLMTCLPIVEGSGDNIFYYARYRASSFEKYGGARKWHDSYNDVTRETARELDVPLIDNWKNIVKKAGGATDEKLIHSRFIDPSGNHLTPKGARIIFEGINKSKIIKTS

SEQ ID NO: 14 - protein, 275 aa, Bacillus licheniformis
  putative lipolytic enzyme (unknown)
MRGFIKWAGLAILCIGLFACTSNRNAENVHYAKDDRQTLDIYTPQSNEGEKHPVLIYLHGGGWTSGDKSRAASKPAFFTDNGYVFVSVNYRLHPDVQYDEMADDAAKAVKWVMDHADEYQIDPSKINVMGHSAGGHLAGLIAADSTYLNRVGLTPAALNSIVVLDGPLDMKRFIEAIPSYKEVFGTDEKVWTKASPLSYINNSKLPPAYLVTRWEDPAVYKFAETANKAKATEFVYQVNSLSHSDLNKMFGSPDAPAEAQNLTKAVMAFLEKENK

SEQ ID NO: 15 - protein, 295 aa, Bacillus licheniformis
  putative hydrolase (ytaP)
MRSQLFKLLGDLPGKKSIDASTLKIEEKSSFILETLLLDLNGIEAVPAYFAKPKQMNGKRPAVLYCHSHGGRYERGKEELVEGAEYLQAPPFAEELTALGFSVLAIDHWAFGDRRGKTESEIFKEMLWTGRVMWGMMVFDSIRAIDYLKMREDIDESRIASLGMSMGGLMAWWTAALDERVSVCVDICAQVDSETLIKTQNLDRHNLYSYVPGLVKHFSTAQIESLIAPRPHLSLVGTADKLTPVQGIDIIEEKLSDVYIEKGAGERYEVFRSHSGHLETAGMRHKAVGFLKTWL

SEQ ID NO: 16 - protein, 259 aa, Bacillus licheniformis
  phospholipase (ytpA)
MWCMEAERPVATIVVIHGACEHHGRYKWLSEMWRSSGFNVVMGDLPGQGTSTRERGHIRSFQEYIDEVDKWVARAKAFELPVFMLGHSMGGLIAIEWFKQQQSGIAGLILSSPCLGLQLKPNKFLDLISKGLNVLAPSMRFESGITPDKATRNKEVIEMDINDSLYITKVSVRWYQEMLKALKSAMEPTDAFLNIPLFVMQAGTDWLVDKKMVVKWFNQLASHNKTYREWDGLYHEIFNEPEREDVFKAARAFAEQYIT

SEQ ID NO: 17 - protein, 266 aa, Bacillus licheniformis
  putative dipeptidylpeptidase (ytmA)
MWIEKRRFPSPNKEVRLFSVTYLSQGLKVKGLLAEPAAPGNYDGFLYLRGGIKNVGMVRPGRIVQFASQGFVVLAPFYRGNQGGEGDEDFAGNDREDAFAAFRLLQKHELVKDGRVHIFGFSRGGIMGILTAIEMKAAAASFVSWGGVSDMVMTYRERKDLRRMMKRVIGGTPEKVPEAYEWRTPFDELEKIEAPVLLIHGANDENVSIEHARRLERELLRRNKSVDTWYFSQYTHYFPPRENRRIVRELSQWMKSRPWTSYGKLK

SEQ ID NO: 18 - protein, 273 aa, Bacillus licheniformis
  2-succinyl-6-hydroxy-2, 4-cyclohexadiene-1-carboxylate synthase (ytxM)
MAVLKLTLKDGVSYKIEDNGLSAEKTAVFLHGFTGSAATWDGIDGYFQGMRLIKLNLLGHGGTDSPSDSRRYTTEKQAADLIEIFDRLNVKQAYLIGYSMGGRLALSLAMIHPERVSGLVLESSSPGLDSPEERKARREQDSRLSRRILEEGIKSFVDYWEGIPLFASQLSMDPSKRKKVREERLNNNPIGLRGSLIGMGTGSQPSWWNKLSGINYPVLLIAGSLDQKFVAINESMAKRIPAARLEIVEHTGHAVHVEEPDFFGRIVSEFILK

SEQ ID NO: 19 - protein, 273 aa, Bacillus licheniformis
  alpha/beta hydrolase (yugF)
MLLTKPSIETIQGVNIYYEHYENPGKTSLVLIHGFLSSSFCYRKIIPLLKDEFNLVAVDLPPFGQSEKSSTFVYTYQNMARVVIELIERLKIKDAVLVGHSMGGQISLYAIKERPELFKKVVLLCSSGYLKRSPRSLIFGSHIPYFYLYIKRWLSKQGVLKNLMNVVYDSSLIDQEMINGYLKPFLDDQIFRALARLIRHREGDLSPDDLKKIETPSLLIWGEEDRVVPVQIGKRLHQDLPNSIFYSLQETGHLVPEENPDYVSDKIANFILT

SEQ ID NO: 20 - protein, 298 aa, Bacillus licheniformis
  dipeptidylpeptidase (unknown)
MTNHIKEINWEQNTNSYIQLIPNIEYTKVEDTSLTLHLLVYRNPMDALFNRKGNQETYPLIIYLQGCGWGWTKQDTSAFIPQLVPFVEQGYVVASVQYRGSGEAVFPAQLHDVKTAVRFLKANAARYNIDPDRVGVWGDSSGGHLALLLGLTEGIEEFEGPDEYRHVSSKVDAVADWFGPVDLLSMSKYPSIFDHDSPNSPESKLIGGAVQENRVQAKQASPISYVHREAPPILIMHGDQDDVVPYQQSVQLFEALIKEGHDALMYKINGAGHNGFTQAHTLDIVKSFFRKHLKPGKA

SEQ ID NO: 21 - protein, 653 aa, Bacillus licheniformis
  putative acylaminoacyl-peptidase  (yuxL)
MKQLITEKDLIKLVSITDPQYSPDGAKIAYVQTKVNEKQDSYDSHIMIYDREKQASVQWTFGKGRNQHPRWSPDGKYLAFTSNREETAQIYVISAAGGEARKVTDIPYDVSQPEWSPDGKSLLCSVKLTKEESVDDEKKTEIEDHEPLEVDSLSYKADGQGFKRGKYTQLVLVQVETGEMKQLTDLERDHFSHAFSPCGDQIAFCANQTDMRVNDVYLMNLSTGDLKRLTGQNGIFSSLSFSPDGKHLAFIGNEKEYQNATLDKAWLYDIETGKLTCLTEMLDVHLSDAVAGDSLVGGVLPKPAWTKDGNGFYVIGSDQGSTGIYYISIEGLAYPVRLEKEHVNGFSLHPDESGFAASIALPVRPSELYHLQLGEEKAERLTDANQAFTEEHIISEPEELQFQAADGLTIHGWLIKPAQYEKGNTYPLILEVHGGPHAMYANAYFHEFQVLAAKGSAVVYVNPRGSHGYGQDFVNRVRGDYGGGDFKDVMAAVDHVLEHYDFVDQERLGITGGSYGGFMTNWAVGHTKRFKAAVTQRSISNWISFYGVSDIGYFFTDWQLGADLFEDPGKLWEHSPLKYADKVETPLLILHGERDDRCPIEQAEQLFTALKKMGKEVKLVRFPNASHDLSRSGHPKQRIRRLEYIAGWFEAYL

SEQ ID NO: 22 - protein, 479 aa, Bacillus licheniformis
  phage putative hydrolase protein (unknown)
MSKKIKKSNLTDETYYRISQRSYNYDYLRKKLKNKEYIRINSSVSGATYWYVDKIKTDEDTGLDAAVLSQAENKNGKWVKSDHPKNVVVAFAGTDPGKDPLSDVEQADINHIVLGNDPKDKTQYVVKKDAKDMSKTFGRYIGSMEQTAMLESGDYKLITKTSQIDQADQLVREVKQKYKGTSTVISTTGHSLGGAEAEYSAVNNDIYAVAFNSPSIVHLHSDEKQKEINNGDYNSYVKSIINPDDMVGAGWWDEFDRHNGTTIYTKDPSIATANREERLDGNKLQQVGRNLLYFANTLIFQNPDTHGINKSNFSFDENGNVQNIEGDELVYDKNLKAMLPPEVASGSGAIKVTPEVAKQLAQKVNAIIDDLRTMKREAENAYQEHDAEINDLKHDTYRQVGHGLYDKLTLEDVNNTLKNLAQSFDKKGNPLFYDVHAEKAYIASLKNTISDLEDISGYLAQIAKDFKSKDKMLANWLKL

SEQ ID NO: 23 - protein, 282 aa, Bacillus licheniformis
  trilactone hydrolase (besA)
MAQEKLQQSVNAAECIPVAIPGTEQRCMRSLDGSRTYRIFIAKPFGEQPPAGYPVIYLLDANSVFGTMVEALRMQSRRPDKTGVGTAVIVGIGYETDAPFDRGRYYDFTLPESGAELPMRPDGADWPEPGGAKAFLSFIEQQLKPEIEQEFTIDVKRQAIFGHSLGGLFVLQTLFTKPDLFQTYIAGSPSIHWHPSFFDKEHDFVSKLQEADGDVKVLLGAGELEKTHKSGMNDNAKRLADRLAALESQGVRAEYHEFKGEGHISVLPVLISRALRFAFKPD

SEQ ID NO: 24 - protein, 220 aa, Bacillus licheniformis
  rhamnogalacturonan acetylesterase (unknown)
MMRDIQLFLAGDSTVCDYDENRAPRAGWGQKIGEFLSAGVLIRNEAASGRSSKSFIEEGRLKRIITQIKSGDYLFVQFGHNDQKPDDRYTEPYTTYKQHLTRYIDEARLKKANPVLITPVQRRSFASDGRFKNTHGDYPDAMIQLAEELRVPLIDLAFKSRQLLESLGPEASKKLYLWLEPGEHPNYPDGARDDTHFCDFGAKEIAKLVAQEAKEAGIPL

SEQ ID NO: 25 - protein, 246 aa, Bacillus licheniformis
  lichenysin synthase (lchAD)
MNEIIKQFGAPASGMQLICFPFAGGYSASFRPLHEHLKTDFDMLAIEPPGHGTNRMALVDNLEKLADLYIAALKPRLSSQFVLFGHSMGGMVVYRLAQKLEQEGIFPAAAVISAIQPPHIQRQKVSHQKDDAFLDHLIQLGGIPKELAEHREMMNFFLPSFRADYRALETFCHTDHTPLRSPVHILNGDRDEKCMRDALGWKKWANTIDFHYFKGGHMYLLSETEQVAAKLKTIAAASRSTAVPTM

SEQ ID NO: 26 - protein, 271 aa, Bacillus licheniformis
  putative esterase (unknown)
MILVQNGVMNMIDGTLIKDTCFGRELLIYLPPSYGSGENRYPVLYVHDLGDLFDPAYSNSLRSIEKLFSSQEVPELLLVGVAPKDRESEYTPFPKRHLLPNKREYGGKGNEYAAFLANRLKPHIDRHYRTNPSREQTGIIGKSFGGLISVYTAFLASETFGKIGSLSGSFWFEGITEFMKNNRFENKHLRIYMDVGSTEGTDRDNLQKEMIPRTKEAFSLLTESAASEKELKFVIDEGGSHRLERFAKRFPEAVKWLFGSCHNSARNGRSF

SEQ ID NO: 27 - protein, 212 aa, Bacillus licheniformis
  spore germination lipase LipC (lipC)
MTLQYTALGDSLTVGVGAGLFEPGFVQRYKRKMEEDLNEEVSLIVFAKSGLETSEILAMLNEPFIMEQVKKADVITITGCGNDLLQSLEIYEKEKDEHVFLEASSHCQKNYSGMLEKIREIKGEKDTRYLVRLLNLYNPFPSIELADKWISGFNRHLKQLESAPQIKVIDTYAVFKGREKEYLSIDRVHPSSRGYEAMSEKLRAAGYGRLEG

SEQ ID NO: 28 - protein, 236 aa, Bacillus licheniformis
  putative esterase/lipase (unknown)
MIGCLCIHGFTGAPYELEPLASHLKQTADWQVEMITLPGHGEMPVLKGVCFQEWIACAEVELIRLLKTCDTVFLIGFSMGGMIAGYLAGKYPVSRLVLLSAAAQYISPGQLIQDLKNVIKDSLQGKLEDNLLYKRYKQKLTSTPVSSAFQFRKLVKMTKPALKNLRIPVLIVQGECDAIVPVSSAYYLYETIPSEEKELCLLPESKHHVCLEENSSILFQTVEAFLKKTPIESIKK

SEQ ID NO: 29 - protein, 626 aa, Bacillus licheniformis
  putative peptidoglycan O-acetyltransferase (yrhL)
MRDQTQQHRYIPGLDGLRAFAVLAVIAYHLNMSWADGGFIGVDIFFVLSGYLITSIILPAYGNDISLTFRDFWVRRIRRLLPAATLMIFATVIWVALFNRELMHTVRGDAISSLLYVSNWWFIFHDLSYFDSFGSPSPLKNLWSLAIEEQFYFIWPIILLLGIYIFKKRERFAAAVVLCALCSAIWMSILYVPGGDPSRVYYGTDTRAFELLIGCALALVWPMKRLSGNRLPNSLKHALHGTELAAFALLMMCIYFVDEFSPFLYHGGMLIISIIAAILIACVSHPSSFLGFALSVKPLRWIGKRSYGIYLWHYPVIVLSTPVQEIGNPVYWHVAMKVAVTFILAELSYRFIEKPIRKDGFKPFVSRVFLDKITEWKTASVPSKISIGLMFAALLVFAGGLSGLADEKKQPQQAYSKSYEETSASADQPADKSEERQDDKKAQTESGNESKTGNGEESSDTKDEQSAEKEPDRIEKEVLAVGDSVMLDIASSLRRKIPGITIDGQVGRQVREALQLTSTYASFNHPDKAVVIELGTNGYFTDGQLDALLDAFSKADVYLVNTRVPRQWESKVNESLRKHADSRQNVTLVDWHAEALQHPEYFTADGVHLMPKGAEALASLIVKAMK

SEQ ID NO: 30 - protein, 303 aa, Bacillus licheniformis
  hypothetical protein (unknown)
MWIGILLMVSGIGISIFAVSLIVSYLFVLPNRSTYEETFFSGLKHGEIIDDVFESHDKEELFLTAGDGCRLHALLFPVQKSRKAVIISHGIKWSLFGGYKYVELFQSIGYNVLLCDSRCHGLSGGSHVSYGFYEKDDLARWADELENRFGKDLWIGVLGESLGAAAAIELMKQDRRIKFCIADSCFSDLTELCRFQLKAAVKLSVPFLIPLASGLIKRRHGWSLADISPVSNLEQSDTPLLIIHGTKDQLVPPEMAKSLYERKKGFKKLYWIEGAGHVGGFRHNPEEYLNKIKDFILKIEPAN

SEQ ID NO: 31 - DNA, 573 bp, Bacillus licheniformis
  putative phosphatase (yhfR)
atgactgcgatttgtttgatccggcacggcgagacggactggaatgctttaggaaaactgcagggaagaacagatatccctttaaatgaaaccggtaaaaaacaagcaaaagaaaccggggcgtttttgaaagggtccgactgggacgtcatcattacaagccctttaagaagagcgaaagaaaccgctgaaatcatcaatcaatacctcggacttgaaatcatcgaaatggaggatttcatcgagaggaattacggagatgcagaaggaatgccattcgaagaaagaatgaggctctatcctgataaagaatatccgaatcaggaatcaaaagaagcgcttgccgaacggctgatggccggcgttcaaaaggtcagtgagcggtatccggacaaaaaagtgctgatcgtcgcacacggagccgcgatacacgcgcttctgtcaaaaatctcaaacggcgacatcaacctggaaaacacgaagctcgtcaatgcctgcctaagcaacatcaaatttcatgaaaataaatggcatgtgaaggattacaacctttcacaccatttgtcagattaa

SEQ ID NO: 32 - DNA, 768 bp, Bacillus licheniformis
  putative peptidase (yitV)
gtgatcataatcgaaaagcagcatatttcaaacattcctcttttgcatattgtaaaagaggagaaaaaagagaaagctcttcctcttgttttcttcatacatggttttacaagcgcaaaagagcataaccttcacttcgcatacctgcttgccgaaaaaggaatgcgcgtgattctgcccgaggctctctatcacggggaaaggaatgaacagctcacccaggaagagctcgccccgagattttgggagatcgtcacgaatgaaatcaaagagctggaagtgctgaaaaaccactttgaacgggaaaacatgattgaaaagggccgcatcggcgccgccggaacatcgatgggcggcattgtcacgctcggcgctttgactcaatacgaatggattacaactgccgtcagtttaatgggcagtcccgcctatgtagaattttttgatcagcagctcgcatttatgcgtgaaaagaaaattgaacttcctatcacagaagaacaaatcgatcagcagcgcgaagaactgaagcgttttgacttgagtctgcagccggacaaattgaacatgaggccgcttttattttggcacggcaaacaggacggcaccgttccatttgctttaacgcgccgcttctatgaatcgattattccactgtatgaagctcggcctgatttgctgcattttatcgaagatgagcgggccggacataaagtatcgagggaaggccttttgaaaacagtcgaatggtttgacgcgcacttgtaa

SEQ ID NO: 33 - DNA, 717 bp, Bacillus licheniformis
  rhamnogalacturonan acetylesterase (yesT)
atgaaacgaacagacaaaaaaccgaaagtgacagtatatttggccggggactcgaccgtcgcagactgtcccccgcatgaagcgccgatggccggctgggggcagatgcttccgctgtttttctcagatgaggctgcggtcgtgaatatggcaaaaggcggggccagttcgaacagcttcattgatgaaggccggctcgatgacattacggagcggcttgcgcctggtgattatgtgttgatccagttcggccacaacgatcaaaaaccgtacggaacagacccttatacaacctatcaggagcatcttgtccgctatgtggaagcggttcgcgaaaaacaagcgacaccggttttgatcacttccgcagagcgcagacgttttgatcaaaacggaaagcccgccgacacactcggggattttccgaaagcgatgaaggcgctcgctgacagcttcgacgttccgctgatcgatttatggtccaaaacaaaagcgctctatgaatctctcggaatcgaaggttcaaaacggctgtttgtccactttcagcctcatgagcaccctcattatccaaatggaatcgaagacaatacccatttttccgaagcaggggcccgtcaagtcgccaggctggtgacagaaggaatcagagagcttggtctgcctctatcggcttatctgctttgtgaaaggagcgatttccgtgccggagtgtaa

SEQ ID NO: 34 - DNA, 1266 bp, Bacillus licheniformis
  putative carbohydrate esterase (unknown)
atgccgaaggagaaaaaatggggacgatcccggccggagcaaagggaagctcagccgaaaaagcgttcaccgtttcggtggaagacgggcgcctcgatctggagtgaaggggcctcagccaaaggtgtatgccatcgaaatttcgaggtgtatcagttcgatttcggtgacgggcctcttgcttcgggttttattccggtaggcgctgcttcggcctatacccctctaaaaggctacggatttgacgacgcggcaaaggtgtcctgcacggaccgaggtacggccgacccgcttcgttcagacttctgcattccggatgggacggcgtttactgtcgatttgcctgacggtcaatatcaagtccaaatcataaccggagatgcgcgggcaaagacgaacatggacgtacgggcaaacggcatgctccagctgtattcgtttggagcggaagcgggcggatacaacgaaaaaacgtttccgattcatatttccggcggccgtctttcgatccaagccttaacggcgagaacaatgccgcgccttaatgcgttggtcattacgaagacggcttatcatgtgacggatgacaacacgacggtctttctggcgggcgattccacggtctccgattatgagcccggctatgcgccgatggcaggctgggggcaatttttaggcggctatttctcggatgaaatcgtcatcgacaaccaggcgaaacgcgcgagaagcgcgaaaagttttgtggatgaacggtcgctggctacgattttgaaccggatgaaacggggcgactatttgttgattcagttcggcattaatgacgcagggcaagatcctgcccgccatacagatccgtatacgacatttcaggaatacttgagaacatatattaacaaagcccgcgaaaaaggaggacgtcatgtgctcgtcacttctcaaagcaaacgtacctatgatgaaaagggcgttttctacaacagcatcggagaatatccgaacgcaatgagacagctgggacgggaaatgaacgttccggtgatcgatttaaacaaaaaaagcattgaatattacaacgagatcggcgttgaagcgactaaaaacgtatttatgtttctggaaccgggagaaagcccgaattatccggaaggcatccaggacagggtgcattttcacgagttcggcgccaaccaattggcaaggctcgtcacaggagaaatctccgatttgaatatatatccgctgaaacactcgctcaagaaataa

SEQ ID NO: 35 - DNA, 645 bp, Bacillus licheniformis
  putative rhamnogalacturonan acetylesterase (yesY)
atgacaatcagcatctatttggccggcgattcgaccgtgcagcattataaggctgattcgcctcagggaggctggggcgaatttctccagtcttatttatccggaaatgtacaggtgatcaaccgggcaatcgggggccggagttcaaaaacgttcgttgaagaaggaagacttgcatctattttggatgtgatcaaacaggatgattggctgttcgttcagatggggcacaatgatgcgtcgaaagacaaaccagaacgctatacagagccgtatacgacgtataaacaatacttgaaaatgtacgttgacggcgcgcgtcaaaaaggggcatcgccgcttctcatcacacctgtcgcaaggctccattacaaagacggcgtctttttaaacgatttcccggactactgcattgcgatgaagcaggtcgcaagcgaagaaaacgtccgcttgatcgatttaatggacaaaagccttcagcattttgcggaaaaaggatacagcgaagtatttaaatattttatgatctcggaaaatgtcaacgactatacacattttacgaaaaaaggggcccgggaaatggcgaagctcgtctcggagggcattcgagagcttgggctgccgtttgtcaaagagacggtatga

SEQ ID NO: 36 - DNA, 1116 bp, Bacillus licheniformis
  hypothetical protein (unknown)
atgtacctgaataaaaagagcgatattcctaaattaacagataaagagtattattttcttagccaaaacacatatagtaccgataaaatgaaagaagcattcaaggaaagaacacctatagagagtaaatcaaataaggctttttttgttgataaaattaaaagggactctgacactggacttgatgcgtatgtttttgttcaagcaaagaaaaaggacggtaaatgggttaaacctaacgcaccagaaaacgtcgtcgtggccttcgctggaacaaatcctaaagaacagttttttcaagatgttattgatgcagacggcggtaacgttgttatgggtcttgatccgaaaaagaaatcgcaatacataatcgaaaaagatgcgaaggatacatcaaaaacaataggaaaatacaacgcaaccccctcacaagacgcgatgcttagcaccggtaaatacaaattaattacgaaaacgtcccaaatcgggcaagctgatgatctggtacgggaagttaaacaaaagtataaaggcacctcgacggttatttcaaccaccggacactctcttggcggcgcagaggctgaatacagcgctgtgaacaatgatatatacgcagtcgcattcaacaatccttccgtcgttaaattgcattctgaagaaaagcaaaaggagatccgaagcggcaaatatgactcgtctgtaaaagcgattgtcaatccggatgatatgactggttccggctggtggaatgagtacgaacgacacgcaggcagaacaatatataccaaagacccctctacttcgcgtgtggaacgacaaatacgactagatccgaagtatagcggcggtatatttggcacagtgtttaatgtcgctgtagactacattgctaccacagcaatgggaatgccggatacacacggcctcaacaaaggcaactttaccttcgacgaaaacggcaacgttcaaaatatagagggcgatgaactcgtttatgacaaaaatttaaaagcgatgcttccaccggaagttgcatccggaagtggcgcaattaaagtaacgcccgaagttgccaagcagctcgcgcaaaaagtaaatgcttgcggacgatga

SEQ ID NO: 37 - DNA, 1017 bp, Bacillus licheniformis
  flagellar motor switch protein (fliG)
atggcaaaaatgaaacaagacaggcttacaggcaaacaaaaagcggccattctcatgatatccttagggcttgatgtgtcagcttccgtttacaagcatttaacagatgaagagatcgaaaggctgacgcttgaaatatcaaatgtcagagctgtatcacctgagaaaaaagatgagattattcaggaatttcatgaaacggctattgctcaagaatatatttctcaaggcggcattcattacgccaagcaggtgcttgacaaagcgctcggcgaagacaaagccgccagcattatcaacaggctgacatcgtctcttcaagtcagaccgttcgattttgccagaaaagcggatcccgagcagattctgaactttatccagcatgagcacccgcagacgatcgcattgattctgtcttatctcgatcccgtgcaatcgggtcagatattgtcagagctgaatcaggatgtgcaggcagaggttgcaagaaggatcgctgttatggacaggacgtcgccggaaatcatcaatgaagtcgaacagattctggagcaaaagctatcgtccacattcactcaggactacacgcagaccggcggtatcgaagccgtagtcgaagtgttgaacggagttgacagaggtacggagaaaacgatccttgacgcccttgaaattcaggatcctgagcttgccgacgagattaaaaagcggatgttcgtctttgaagatatcgttacgcttgataaccgcgctattcaaagggttatccgcgatgtggaaaacgacgatctgcttctttctctcaaagtggccagcgaagaggtcaaagacatcgtcttcagcaacatgtcgcagcgtatggctgaaacatttaaagaggaaatggaattcatgggccctgtgcgtctgcgtgacgtagaagaagctcaatcgagaatcgtaagcgtcatccgaagacttgaagaagcaggtgagatcgtaatcgccagaggcggaggagatgatatcattgtctaa

SEQ ID NO: 38 - DNA, 861 bp, Bacillus licheniformis
  AB hydrolase superfamily protein (yisY)
atgttcagccctcggagacattcacctatttacatcttgggagtatcttatggtaaagacatgatcgggggcgacggcatgggatattttatcgaggcagaaccaagcgttaatctgtacgttgaagacatcaatcctcaaagcggcaaagccattgtatttttgcacggctggccattaagccatctccagtttgaatatcaatttgacgatctttcgcgaaagggataccgctgtatcggcattgactggcgtggattcgggcgttcggataagccggcgacgggctataattacaaccgtttggctgatgatatccgcgctgtcgttgaggcgcttcaattaaacaacttcacgcttgccggtcattcaaccggcggcgccatagcactccgctatatgtcccgatataggggcgctggggtctcaaagctggttttgatcgatgcggcggcgcctgtgggatttactgaagaaacggcagccaaattgcttcagcaagcgtctaacgatcgtcccaagatgatgcgcgaagtgaccgacacgtttttctttcaatatattacacaaccgttctcagactgtttttttcaattgggattgcaggcggcaggctggtcgacaaaagcggtcatcgaaatgctgagagatgagaaactgtatgatgatccggaaaaagtcgctgctccagccttaatcattcacggtattcaagacaaggtgattccgtttgcacaagcaaaggagctcagccgcaaaatcagaaattcatatcttgttccgtttcaatacagcggacacggtccattctgggaggaacgcaaaatgttcaatcggattttaacgcaatttatcggttga

SEQ ID NO: 39 - DNA, 606 bp, Bacillus licheniformis
  putative hydrolase (mhqD)
atgaattatatatatgaaaaaggggactctgatctcaccctgttgttattgcatggaacagggggaaacgaacacgatcttctgccgatcggcaagatgattgatcccgatgcaaatttgctcggggtcaggggaaatgtgctggaaaacggaatgccccgttttttcaggcggctgagagaaggggtgtttgacgaaaaagatttgatcgaacggacggatgaattaaaaacgtttctagatgaagcagccaaggaacttcactttgacagaagccgggtggtcgcaatcggctattcaaacggcgccaatatcgcgggaagtctgctgttccgtcatgaacatgtactgaaaggggcgattttgcatcatccgatggttccgtttagaaatcgcaagctccctgatatgacaggtctgcccgtgtttatcggggccggcaaaacagatccgctctgtccgccgcaggaatcaagcgagcttgccggctatttcacaaacgccggcgccaaagtgctccttcactgggaaggcggaggccatcagctgacacacggcgaaattgaaaaggcgaaaatctggtatgaaaagcatataaaagcataa

SEQ ID NO: 40 - DNA, 753 bp, Bacillus licheniformis
  putative esterase (ypmR)
ttgaacatacgttttattacagtcatgatggccctcgtctgtctgctttcagcctgtacggagtggaatgcgggtgttgaaaaaacatcggtctcccctaagcgcgatatcgtgattgcagcggtgggcgattctctgacagagggggtaggcgatcaagaaaagaaaggctatgtcggcatggtggccgatgagcttgaaagccggagcgatgtgaagtccgtaacggtcaaaaattatgccgttaaaggctcccggactgatcagctgcttgaaagattaaaagacaaagaagtacaagagggcctgaaagatgcagactacattttgtttacaatcgggggcaacgacctgatgaaagtcgtccgccaaaacttcgcgcacttaacgctcacacctttccgtgctgaacaaaaactgtttgaaaagcggttttcgaatattttagcggaaatcagagagcaaaacgccagcgctgaattgatttatgtcagcatgtataatccgtttaagtttacgctgtctgaactgcgggaagttgaccaagtcgtagacgaatggaatgaaggcgcggaaaaaaggctgaagaaggtctccaacacgaaaatggcggatatcgctgatatttttgaggaatacagcgatgaaaagaagattgcagaagacgagtttcatcctaatcaatacgggtattctttaatagcgaaacgcgtttacgaacaaattaaaaacgaagacctcccagcagaataa

SEQ ID NO: 41 - DNA, 615 bp, Bacillus licheniformis
  putative lipase (lip)
atgcgtcgtcattcatttttatctattctattgatttgcatgctgtctgttgtgtccgtattttcgttccggccttcagcagcttccgccgcttcccacaatccggtcgtcatggtccacggcatcggcggagccgattataacttcatcggcattaaatcgtatttacaatctcaaggctggacaagcagtgagctttacgccatcaactttatcgataaaacgggaaataatataaacaatgctccgagattatccgaatacatcaagcgtgtgctgaatcaaacaggagcatcaaaagtcgatattgtcgcccacagcatgggcggggccaatacgctctattatattaaaaatctggatggtgcggataaagtcggacatgtcgtcacccttgggggcgctaatcgtctcgttacaaacacggcgcctcagaatgacaaaatctcatacacttcgatttacagcacaagcgactatatcgtcttaaacagcctctccaaacttgatggtgcaaacaatgtgcaaatctcaggcgtaagccatgtcggtcttttgttcagcagcaaagtaaatgccttgattaaagacgggctgaccgccagcgggaaataa

SEQ ID NO: 42 - DNA, 762 bp, Bacillus licheniformis
  hypothetical protein (yqiL)
atgaccataacggaaagaatatatactgttgaagggtgcatgttttatacaaagcagagaaaaggaaaaaagaacgcaacgattatttttgaagcgggctacggtttttcttcaacgacttggcagcccattttatccgatatagacgaagatctcggcttgtttgcatacgaccgtgcaggccttggcaaaagcgggccgagcaccagcaaacgaacagcggatcagatggtgaaagaactaaaagcgctgcttgcagcagcagacgtcaaaccgccctacctgtttgtcgcccattcgttcggcgccgtcattgcaaggctgtttgcaagccggtacagcgatgaagtcatcggcatggtgctgcttgatcccgcttgtgaagatcaggaggaaaaagtgctgccgctacttccggacacattaaaagaagattatgtcaaacagttttcgatcgaagggtcccatgaggactttaaacaaagcctggacatgctgaagcaggaaaagaaacacctcggttcggttcctttgctcgtcctctcggcgggaaaaaggacggaggcggcgaagcaagctcatcaagaatggctgaagcttcacagcagactgctgtctctatcgcatcaaagcggctggattcaggcgaaaaacagctcgcatttgatccatcatgatgagccgcacatcgtccagcttgcgatctatgatgtgtggtatgcggcacagcagcaaacatacgcctatcaagccgccaattaa

SEQ ID NO: 43 - DNA, 732 bp, Bacillus licheniformis
  putative lipoprotein (yqeF)
atgaaaaaaagcttggtgatggttctcgtttttctgcttctgctttcaggctgcggagaggacgatcccagaaaagtcgtagcatttggagacagcaatacacgcggctcaaattggcaatttcgcgattatccaaaagcggaaaagtgggtcaatttacttcagacttcccttcaagggcagtacaaaataaataatgcaggaataggcggagaaacgacggaagacgccaggtttcggttcaggaaggatgtgctggacaagcagccggcatatctttttatcatgttcggaacaaatgatgcagcaattcttacgaaagggctgccgcgcgtatccaaaaaaaggtttaaagaaaacctttattattttgttaaagaaagccgccgccgcgggatagagccggttttaatgacgtgcctgccgatcgtcgaaggaagcggggacaacatcttttattatgcaagataccgggcttcttcgtttgaaaaatacggtggagcgcgtaaatggcacgattcctataatgacgtcacaagggaaacggcgcgggaacttgatgttccgcttatcgacaactggaaaaacatcgtgaaaaaagcgggcggagctacagatgagaagctgatccattcgaggtttatcgacccatcgggaaatcacttaaccccaaaaggcgcgcgaattatttttgaaggaattaacaaaagcaaaatcataaaaacgtcttga

SEQ ID NO: 44 - DNA, 828 bp, Bacillus licheniformis
  putative lipolytic enzyme (unknown)
ttgagaggattcatcaaatgggcaggattggcgattctgtgtattggcttgtttgcgtgtacttctaacaggaatgccgaaaatgttcattatgcaaaagacgacagacaaacattggatatttatacaccgcagtcaaacgagggcgaaaaacatcccgttctcatctaccttcacggcggaggatggacaagcggcgacaaaagcagggctgcttcgaagccggcattttttacggataacggctatgtttttgtatctgtcaactatcggcttcatcccgatgttcagtatgatgaaatggctgatgatgcggccaaggccgtaaaatgggtgatggatcatgcggacgagtatcaaatcgatccgtcaaaaatcaacgtcatgggacattcggcagggggacacttggccggattaattgccgcagattcaacatatttaaatcgtgtcggcctcacgccggccgcactcaattcgattgttgttttagatggccccttggatatgaaacgattcatcgaagcgatcccgtcttataaagaagtgtttggcacagacgaaaaggtgtggacaaaagcatcgccgctctcatacataaacaactcaaaattgccgccggcctatcttgtgacccgctgggaagaccctgccgtttacaagttcgcggaaaccgcaaacaaagcgaaggcaacggaatttgtgtatcaagtcaacagcttatctcacagtgatttaaacaagatgttcggctctccagatgcaccggcagaagcgcagaatctgacaaaagcggttatggcgtttttagagaaggaaaataaatag

SEQ ID NO: 45 - DNA, 888 bp, Bacillus licheniformis
  putative hydrolase (ytaP)
atgcgcagtcaacttttcaaactgctcggcgatttgccggggaaaaaaagcattgatgcgagcactcttaaaatcgaagaaaagtcatcttttattctcgaaacgcttttgcttgatttaaacggaattgaagcggtgccggcctattttgcaaaaccgaaacagatgaacggcaagcggccggccgtgctttactgtcattcacacggagggcgctacgaaaggggaaaggaggagctggttgaaggagcggagtacttgcaagcgccgccgttcgccgaggaattgacggctttgggattctccgtgcttgcgattgatcattgggctttcggagacaggcggggcaaaacggagagtgagatatttaaagaaatgctttggacagggcgggtgatgtggggaatgatggttttcgacagcatcagagccatcgattatctgaaaatgagggaggacatagacgaaagccgcatcgcttcactcggcatgtcgatgggaggtctgatggcctggtggaccgctgcattggatgagcgggtcagcgtatgcgttgatatatgcgctcaggtggacagcgaaacattaatcaagacgcagaatttggaccgccacaatctctattcttatgttccagggctggtcaaacacttttcaaccgctcaaatcgaaagcttgatcgctccaaggcctcatttaagcctggtcggaaccgcagataaactgacgccggttcaagggattgacattattgaagaaaagctgtccgatgtgtatatagaaaaaggcgcaggtgaacgctatgaagtgtttcgttctcattcagggcacctcgagacagccggaatgaggcataaagcggtcggtttcttaaaaacatggctgtaa

SEQ ID NO: 46 - DNA, 780 bp, Bacillus licheniformis
  phospholipase (ytpA)
atgtggtgcatggaagcggaaagacctgttgctacaatcgtcgtcattcacggtgcatgtgagcatcacgggcggtataaatggctgtccgaaatgtggcgttcatccggctttaatgtcgtgatgggggatttgcccggccagggcacttcgacaagagaaagaggacatatacgctcttttcaagaatatattgatgaagtggacaaatgggtcgccagggcgaaagcatttgaactgcctgtgtttatgctcgggcacagcatgggcggcctgattgcgatcgaatggtttaagcagcagcaaagcggcattgccggtctcattttatcatccccttgtctcgggctgcaattaaagccgaacaaatttctggacttgatttccaaggggctcaacgttttggcgccgtctatgcggtttgaatccgggatcacgcctgataaagcgacgagaaacaaagaagtcatcgaaatggacatcaacgattcattatatatcacaaaagtttctgtcagatggtatcaagaaatgttaaaagcgttgaaaagcgcgatggagccgacggacgcctttctcaacatcccgctctttgtcatgcaggcgggcaccgactggcttgtcgacaaaaaaatggtcgtcaagtggtttaaccagcttgcttcccacaataagacgtacagggaatgggatggcttgtaccacgaaattttcaatgagcctgaacgggaagacgtttttaaagcggccagagcgtttgcggagcagtatataacataa

SEQ ID NO: 47 - DNA, 801 bp, Bacillus licheniformis
  putative dipeptidylpeptidase (ytmA)
atgtggatagaaaaaagaaggtttccatcgccgaacaaagaggtgcgtctgttttccgtcacatacctttcacaaggactcaaggttaaggggcttttggccgaaccggccgctcccggaaattatgacgggtttttgtatttgcgcggcggaattaaaaacgtcgggatggtcagaccgggccgaatcgtccagttcgcttcacagggctttgtcgttcttgctccgttttacaggggcaatcaaggaggagaaggcgatgaggattttgcaggtaatgaccgggaagatgcttttgccgcttttcgtcttcttcagaagcacgaactggtaaaggacggacgcgttcacatttttggcttttcccgcggcggaatcatgggcatcttaacggcaatcgaaatgaaagcagcggccgcttcgtttgtgtcttggggcggtgtcagcgacatggtcatgacctatcgcgaacgaaaagatttgcggcggatgatgaagcgggtcatcggcgggacgcctgaaaaggttcccgaagcgtatgaatggaggacgccttttgacgagctggagaagattgaagcgcccgtgcttttgatccacggagcaaacgatgaaaatgtctccatcgagcatgcgcggcgtcttgagcgtgaacttctgcgaaggaacaagtctgtggatacgtggtatttcagccaatatacccactattttcctccgcgcgaaaacaggcggattgttagagaattatcccaatggatgaaaagccgtccttggacaagttatggtaaactaaagtaa

SEQ ID NO: 48 - DNA, 822 bp, Bacillus licheniformis
  2-succinyl-6-hydroxy-2, 4-cyclohexadiene-1-carboxylate synthase (ytxM)
gtggccgttttgaaattgacactgaaagacggcgtttcctataaaattgaagacaatggcctgtctgcagaaaaaacggcggtgtttttgcatggctttacaggaagcgcggccacatgggacgggatagacggctattttcaggggatgcgtctgatcaagctgaatctgctcggacacggcggaaccgactcgccatcggacagccggcgctacacaacagagaagcaggcagcggatctcatcgaaatttttgatcgtttaaatgtaaaacaggcttacctcatcggttattcaatgggcggccggcttgccttatccttggcgatgattcaccccgagcgtgtgtccggtcttgtgctggagagcagttctccggggcttgattcgcctgaagaaagaaaagcgcgcagggagcaggacagccggctcagcaggcggatacttgaagaaggaatcaagtctttcgtagattattgggaaggcattccgctgtttgcatcccagctttcaatggacccttcaaaaagaaaaaaagtgcgcgaagaacgtctgaacaacaatccgatcggccttcgaggcagcttaatcggaatggggaccggatcacagccgtcttggtggaacaaactgagcgggatcaattatccggtgcttctcatcgcgggaagcctggatcagaaatttgtcgccatcaatgaaagcatggcaaagcggattccagccgcccgcctcgagattgttgaacataccggtcatgcggttcatgtggaagagcccgatttttttggtagaatagtaagtgagtttattttaaagtag

SEQ ID NO: 49 - DNA, 822 bp, Bacillus licheniformis
  alpha/beta hydrolase (yugF)
atgctgttaacaaaaccttctatcgagaccattcaaggggtcaatatatattatgaacattacgagaatccaggcaagacatcgctcgttttaatacacggttttctttcttcctcgttttgctacagaaaaatcattcctcttctgaaggatgagtttaatttagtcgccgttgacctgcctcccttcggccaatcagaaaaatccagcacctttgtgtatacctatcaaaacatggccagggtggtcatcgaactgatcgaaaggctgaagattaaagacgccgtgcttgtcggtcattcgatgggcggccagatttcactttatgcgataaaggaaaggcctgaactgttcaaaaaagtcgtcctgctgtgcagctcaggctatttaaagcgttcgccccgctcgcttattttcgggagccatattccttatttttatttgtacatcaagcgctggctgtcaaaacagggcgtcctgaaaaacctcatgaatgttgtctacgacagctccctgatcgatcaggaaatgatcaacggctacctgaaaccgtttctcgatgatcaaattttcagggcgctcgcacgtcttatcagacaccgggaaggcgatttatcaccggatgatttgaagaaaatcgagacgccttccctcttgatctggggcgaagaggaccgcgtcgtcccggtacagatcggaaaaaggcttcatcaggatctgcccaattccattttctactccctgcaagaaaccggacacctcgtccctgaagaaaatccggattatgtatcagacaagattgccaatttcattttgacatga

SEQ ID NO: 50 - DNA, 897 bp, Bacillus licheniformis
  dipeptidylpeptidase (unknown)
atgacgaatcatataaaagaaatcaactgggagcaaaacaccaactcttacattcagctcataccgaatattgagtacacaaaagtggaagacacttctttgacattacatttgctggtgtaccgcaatccaatggatgcgttatttaacaggaaaggcaatcaggaaacatacccgctgatcatttatttgcaagggtgcggatggggctggacaaaacaggatacatccgcctttatcccccaactggttccatttgtcgagcaaggatatgtcgtggcaagcgttcaatacagaggcagcggagaggcggtgtttccggctcagcttcatgatgtgaaaacagcagtccgctttttaaaagcaaatgccgcccgctataacatcgatcctgatagagtcggagtctggggggattcttcaggcggccatttggctcttttgcttggattgaccgaaggaattgaagaatttgaaggcccggacgaataccgtcatgtatcgagcaaagtcgatgcggttgcggactggttcggtccggtcgaccttctgtccatgagcaagtatccgtccatcttcgatcatgattcacccaattcaccggaatccaagctgatcggcggagctgttcaagaaaacagagtacaggcgaaacaagcgagtccgataagctatgttcacagggaagcgcctcccattctgattatgcacggggatcaggacgatgtcgtcccttatcaacaaagcgtccaattgtttgaagccttaataaaagaaggccatgatgcccttatgtataaaataaacggggccggacataacggttttacccaggctcataccctggacatcgtcaaatcgttcttccgcaaacatttaaagcctggaaaagcctga

SEQ ID NO: 51 - DNA, 1962 bp, Bacillus licheniformis
  putative acylaminoacyl-peptidase  (yuxL)
atgaagcagctgataaccgaaaaagacctcatcaagcttgtatccattaccgatccccagtattcgcccgatggtgcaaaaatcgcctacgtccaaacaaaagtaaacgaaaaacaggactcatatgattcgcatatcatgatctacgaccgcgaaaaacaagcttcggtgcaatggacgtttgggaaaggcagaaaccagcatccccgctggtcgccggacggcaaataccttgcatttacgtccaatcgcgaggaaaccgcacaaatttacgtgataagcgccgccgggggagaagcgagaaaagtgaccgatattccctatgatgtatcacagcctgaatggtcgccggacggcaagtcattgctttgttcagtgaagctgacaaaagaggaaagcgtcgacgatgagaagaaaaccgagattgaagaccacgaaccgctggaagtcgattctttgtcatataaagcagacggccaagggtttaaaaggggaaagtatacgcagcttgtcctcgttcaagtggaaaccggggaaatgaaacagctgaccgatcttgaacgcgaccatttcagccatgcattttcaccgtgcggagatcagattgcattttgcgccaatcaaacggatatgcgtgtgaacgatgtctacttgatgaacctttcgacaggtgatttgaagcgcctcacaggtcaaaacggcattttttcttcactatcgttttctcctgacggcaaacaccttgcatttatcggaaacgagaaagaatatcaaaacgccacgctcgataaagcatggctgtatgatattgaaacaggaaaactaacgtgtctgacggaaatgcttgacgtccatctcagcgatgcggtggcaggagacagccttgtcggaggcgttcttccgaaaccggcctggaccaaagacggaaacggcttttatgtcatcggatctgaccagggttctacaggcatttactacatatcaatcgaaggtcttgcttatccggtccggttggaaaaagagcatgtgaacggattcagcctgcatccggacgaaagtggctttgcggcatccatagccctgcccgtccggccgagcgaactttaccatctccaattgggtgaagaaaaagcggagcgactaacagatgccaaccaagcgtttacggaagaacatatcatatcggaaccggaggagcttcaatttcaggcagcggacggtctgacaattcacggatggctgataaagcccgcccaatatgaaaaaggaaacacgtatccgctcattctggaagtgcacggcggacctcacgccatgtatgcgaatgcttattttcatgaatttcaagtgctggccgcaaagggaagcgccgtcgtatatgttaacccgcggggaagccacggctacggacaggattttgtcaaccgggtgaggggcgactacggcggtggagattttaaggatgtaatggcggctgttgaccacgtgcttgaacattatgatttcgtcgatcaagaaaggctcggcatcaccggaggaagctacgggggctttatgacaaactgggctgtcggccacacgaagcgcttcaaggccgcagtgacacagaggtccatttccaactggatcagtttttacggggtaagcgacatcggctattttttcacagactggcagctcggtgcagacctttttgaagatccaggcaaactgtgggagcattcgccgctcaaatacgcggacaaggtggagacccctcttctcattctgcatggcgaacgggacgacaggtgcccgattgaacaggcggaacagctgttcacggcgctgaaaaaaatggggaaagaagtaaaactcgtcagatttccgaacgcgtcacacgatttatcaagaagcgggcatccgaagcagcggatcaggcggcttgagtatattgcaggctggtttgaagcttatctataa

SEQ ID NO: 52 - DNA, 1440 bp, Bacillus licheniformis
  phage putative hydrolase protein (unknown)
ttgagtaagaagattaagaagtccaatcttacggatgagacttattatcgtataagccaacgttcttacaactacgattatctcagaaaaaaattaaaaaacaaagagtacatacggataaattcatctgtttccggagccacctactggtatgttgataaaattaaaacagacgaagacaccgggttagatgcggccgttctatcccaggccgaaaataaaaacggcaagtgggtgaaatccgaccaccccaaaaacgtcgttgtagcttttgcgggaactgatccggggaaggacccgttaagtgacgtagagcaagcagacattaatcatatcgtcttaggaaacgatccgaaagataaaacacaatatgtcgtcaagaaagatgcgaaagatatgtctaagacgttcggtagatatattggttcgatggaacaaactgccatgcttgagtccggagattataaattaattacaaaaacatcgcaaatcgatcaagccgatcaacttgtgcgggaagttaaacaaaagtataaaggcacctcgacagttatttcaaccaccggacactcgctcggcggcgcagaggctgaatacagcgcggtcaacaatgatatctatgccgtcgcgtttaatagcccctcaatcgttcacctgcattctgatgaaaaacagaaagaaattaataacggagattataactcttatgtaaaatctatcattaatccggatgatatggtcggtgccggttggtgggacgaattcgatcgccacaatgggactaccatctacacaaaagacccttctattgcaacggctaatcgcgaagaaagacttgacggcaataaactccaacaggttggaagaaaccttctgtatttcgctaatacactaatctttcaaaatccggatacacatgggataaataagtcgaatttctctttcgacgaaaacggcaacgttcaaaatatagagggcgatgaactcgtttatgacaaaaatttaaaagcgatgctcccaccggaagttgcctccggaagtggcgcaattaaagtaacacccgaagttgccaagcagctcgcgcaaaaagtaaatgcgatcatcgatgacttgcggacgatgaaacgggaagccgaaaacgcttatcaggaacacgacgccgagatcaacgacctgaagcacgatacctaccgtcaggtcggccacggtttatacgacaagctgacactcgaggatgtaaacaatacgctgaaaaacctggcgcagtcgtttgacaaaaaaggcaatccgctattttacgatgttcatgccgaaaaagcgtacatcgcctcattaaaaaatacgatttcagatttagaagacatcagcgggtacctggcgcagattgcaaaagattttaaatcgaaagataaaatgctggcaaattggcttaaactttag

SEQ ID NO: 53 - DNA, 849 bp, Bacillus licheniformis
  trilactone hydrolase (besA)
atggctcaagaaaaacttcagcagtctgtcaatgctgctgaatgcatcccggttgcgataccggggacagaacagcggtgcatgcgttcactggatgggagccgcacatatcggatttttattgcgaaaccatttggggaacagccgcccgcaggatatcccgtgatttatctgcttgatgccaattccgtgtttggaacgatggtggaggcgttgcggatgcagtcgcgccgtcccgataaaaccggtgtagggacggctgttattgtcggcatcggttacgagaccgacgctccctttgaccgggggcggtattatgattttactctgccggaatcgggcgctgagctgcccatgaggcccgatggagcagactggccggaaccaggcggggcgaaagcttttctgtccttcatcgaacagcagttaaagccggagatcgaacaggaattcacgattgatgtgaaaaggcaggcgattttcggtcattccctaggcggactttttgtcctgcagaccctgtttacaaaaccggatctatttcagacatatatcgccggcagtccgtccatccattggcatccgtcattttttgataaagagcatgactttgtttcgaagttgcaggaagctgatggcgatgtaaaggtattgcttggggccggagaactcgaaaagacgcataaaagcgggatgaacgacaatgcaaaaaggctggccgaccgcttggccgctttggaaagtcagggggttcgcgcggagtaccatgaatttaaaggggaaggtcatatatcggttctgcctgtgttaatcagcagagcgctgcggtttgccttcaagcctgattga

SEQ ID NO: 54 - DNA, 663 bp, Bacillus licheniformis
  rhamnogalacturonan acetylesterase (unknown)
atgatgcgagacattcagttgtttttggccggggactcaacggtttgcgattatgatgagaatagggcgccgcgtgcaggctgggggcagaaaatcggcgagtttttaagcgcaggtgttttgatccgcaatgaagcggcatcaggaagaagctcaaaaagctttattgaagaaggcaggctgaagcgaatcataacacagatcaaaagcggcgattatttgtttgtccagttcggtcataacgaccaaaaaccggatgatcgctatacagagccatacacaacttacaagcagcatttgacccggtatatagatgaggcacggttaaagaaagccaatcctgttctgattacaccggtccagagaagatcatttgcaagcgatggccgctttaaaaatacccatggcgactatcctgatgcgatgatacagcttgcggaagagcttcgggtgccgcttatcgatctcgccttcaaaagccgtcaattgcttgaatcgctaggaccggaagcttccaaaaagctgtatttatggcttgaaccgggggaacaccccaactatccggacggagccagggatgacacccacttttgcgacttcggggcaaaagaaatcgctaagcttgttgcacaggaagcaaaagaagcgggaattcctttataa

SEQ ID NO: 55 - DNA, 741 bp, Bacillus licheniformis
  lichenysin synthase (lchAD)
atgaacgagatcattaagcagtttggagcgccggcaagcggtatgcagttgatctgtttcccgtttgccgggggatattcggcatcattcagaccgcttcacgaacatttaaaaacagattttgatatgctggcgatcgaaccgcctggacacggcacaaatcgaatggcgcttgtcgacaatttggaaaagctcgccgatttgtatatagcggcgcttaagccgcggctttcctctcaattcgtgctgttcggccacagtatgggcggcatggttgtttacaggctggcgcaaaagcttgaacaagaagggatctttccggcagccgctgtgatatcagcgatacagccgccccatattcagcggcaaaaggtgtctcaccaaaaggatgacgcatttctcgaccatctcattcagcttggcggaatacccaaagaactggcagagcacagggaaatgatgaactttttcctgccttcgttcagagcggactacagagcgcttgagacgttctgccataccgatcatacgccgcttagatcgcctgtgcatattctgaacggagaccgcgatgagaaatgcatgagggatgcgcttggctggaaaaagtgggcgaatacgatcgatttccactatttcaaaggcggacatatgtatcttttatcagaaacggaacaagtagccgccaagttgaaaacgattgccgcggccagccgcagtacggctgttccgacaatgtaa

SEQ ID NO: 56 - DNA, 816 bp, Bacillus licheniformis
  putative esterase (unknown)
atgatacttgtgcagaatggagtaatgaacatgatagacggaacattgataaaagatacatgcttcgggagggagcttttgatctatcttcctccttcatacgggagcggtgaaaaccgctatcccgttctatacgtccatgacctgggagatttattcgatccggcatacagcaacagtctgcgctcaatcgaaaagctgttttcgagtcaggaagtgccggaattgctgctggtcggagtggctccaaaagacagggaaagcgaatacacgccttttcccaagcggcatttgctgccgaataaacgggagtacggcgggaagggcaacgaatatgccgcttttttggccaatcggctcaagcctcatatcgaccggcactaccgaacaaatccgagccgtgaacaaacaggaatcatcggaaagtcgttcggagggttgatctcggtttacacagcttttctagcatcggagacctttggaaaaatcggatctttgtcaggttctttctggtttgaaggaataaccgagtttatgaagaataaccgctttgagaataagcatctgcgcatttatatggatgtcggaagcacggaagggacagacagggataatctgcagaaagagatgataccgaggacaaaggaagccttcagcctgttaacagaaagcgccgcctccgaaaaagaactgaaatttgtcattgatgaaggtggctcacatcggcttgagcggtttgcgaagcgttttccggaagccgttaaatggctgtttggctcctgtcacaacagcgcacggaatggcaggagtttctga

SEQ ID NO: 57 - DNA, 639 bp, Bacillus licheniformis
  spore germination lipase LipC (lipC)
atgacgcttcaatatacggctctcggcgactctttaaccgtcggggtcggtgcaggtttgtttgagccgggatttgtacagcgctataaaaggaaaatggaagaggatttgaacgaagaagtttctttgattgtatttgcgaaatccggtcttgagacgtcggaaattttagcgatgctgaacgagcctttcatcatggaacaggtcaaaaaagcggacgtcatcacgataacgggctgcggaaatgatcttctgcaatcgctggagatatatgaaaaagaaaaggatgaacacgtctttttggaagcttcctcccactgtcagaaaaactactcaggcatgcttgagaagatcagagaaataaagggagaaaaagatacgcgttatctcgtccggcttctcaatttatacaacccatttccgtcaatcgaattagcggacaaatggatttccggctttaacaggcacttaaagcagctagagtccgctccgcaaatcaaggtgatcgatacttatgctgtctttaaaggacgcgaaaaagaatatttgtcaattgaccgtgtgcatccaagcagcagagggtatgaagcgatgtcggaaaagctgagagcggcgggctacgggcggctggagggctga

SEQ ID NO: 58 - DNA, 711 bp, Bacillus licheniformis
  putative esterase/lipase (unknown)
atgataggatgtttatgcattcacggatttaccggagcgccgtatgaattggaaccgctggcttctcatttaaaacaaacggcagattggcaagttgaaatgatcacgctccccgggcacggtgagatgccggttttaaaaggcgtctgttttcaggaatggatcgcttgtgctgaagtggagctcatccgcttgttgaaaacgtgtgatacggtatttttaatcggattttcaatgggcgggatgattgccggctatttagccggaaaatatccggtcagccgccttgtcctgttgagtgcggccgcccagtacatcagcccgggacagctgatccaggatttgaagaatgtgatcaaagattcactgcaaggcaagctagaggataatctgctttacaaaaggtacaaacaaaagctgacaagcactccggtgtcttctgcatttcagtttcgcaagctcgtcaagatgacaaagcctgcattgaaaaacctccggatccctgtgctcattgttcagggggagtgtgatgccatcgtgccggtgtcaagcgcgtattacttgtatgaaaccattccttccgaagaaaaagagctgtgcctcttgccggaatccaagcaccatgtctgccttgaagaaaactcctctatcctatttcaaaccgttgaggcgttcctcaaaaaaacgccgattgaatctataaaaaaataa

SEQ ID NO: 59 - DNA, 1881 bp, Bacillus licheniformis
  putative peptidoglycan O-acetyltransferase (yrhL)
atgcgagatcagacacaacaacaccgttacataccggggttagacggccttcgggcttttgccgttttggcggttatcgcttaccacctcaacatgagctgggccgacggaggcttcattggggttgatatcttttttgttttatcaggatatttgattacatcgattatcctgccggcgtatggaaatgacatttccctcacattccgcgatttttgggtgcgccggatacggcggctgcttccagcggcgacgctcatgattttcgcaaccgtcatatgggtcgctttgtttaacagagagctgatgcataccgtacggggagatgcgatctcctctcttttgtatgtcagcaattggtggtttatctttcacgacctgtcatattttgatagtttcggatcgccgtctcctttaaaaaacttgtggtcactggcgattgaagaacagttttattttatctggccgatcattttgctgttgggcatctacatatttaaaaagcgggagaggtttgcagcggcggttgtgctgtgtgcgctttgttcagccatatggatgagcatcctgtacgtcccgggcggcgatccgagccgtgtatattacgggacggatacacgcgctttcgagctgttgatcggatgcgcattagcccttgtgtggccgatgaaaaggctgtctgggaacaggctccccaacagcctcaagcatgcattgcacggaactgagcttgccgctttcgcgctgcttatgatgtgcatttattttgtcgatgaattcagcccgtttctctatcacggcggaatgcttataatcagcataatcgcagctattttgattgcatgcgtcagtcatccgagcagcttcctgggctttgccctttccgtgaagccgctgcgctggatcgggaaaaggtcgtacggcatttatctttggcactatcccgttatcgtgctgagcacgcctgttcaggagattggaaatccggtgtactggcatgttgcgatgaaggtcgccgttacatttattctggcggaactttcgtatcgctttattgagaagccgatccgaaaagacggttttaaaccgtttgtcagccgggtttttctggataagatcacggaatggaaaacggcttccgttccaagcaaaatatcaatcggcctgatgtttgccgcgctcctcgtttttgcagggggtctgtcgggattggccgacgaaaagaaacagccgcagcaggcctattcaaaatcttatgaggaaacaagcgcctcagcggatcagccggctgacaaaagtgaagaacggcaggacgataagaaagcacaaactgagagcggaaatgagagtaaaaccggaaacggggaggaatcgtcagatacgaaagatgagcagtctgcggaaaaagagccggatcgcatcgaaaaagaagtattggctgtcggggattcggtcatgctggacatcgcttcaagcctgcgccggaagattccgggaatcacaatcgacggacaggttggaaggcaagtgagggaagcgcttcaattgacatcaacctacgcctcctttaatcatccggacaaagccgtcgtcattgaattgggaacaaacggctattttacggacggccaattggacgctttgcttgatgctttctcaaaagcggatgtctatctcgtgaatacgcgcgtcccccgacaatgggagagcaaggtgaatgaatcgcttcggaagcatgcggacagccgtcaaaatgtgacattggtcgattggcacgctgaagcactgcaacatcccgaatatttcaccgccgacggcgtccacctgatgccgaaaggtgctgaagccttggcatcgcttatcgtcaaagcgatgaaataa

SEQ ID NO: 60 - DNA, 912 bp, Bacillus licheniformis
  hypothetical protein (unknown)
gtgtggatcggaattctgttgatggtctctgggattgggatcagtattttcgccgtcagtctgattgtcagctatttgtttgttttgccgaaccggagcacatatgaagaaacttttttcagcggactcaaacacggcgagatcatcgatgacgtgtttgagtcccacgacaaagaagagctgtttttgacagcaggggatggatgccggctgcatgcgctgctatttcccgttcaaaagagcaggaaagccgtcatcatctcccacggcatcaagtggtcgctgtttggcgggtataaatacgtcgagctgtttcagtccatcggctacaatgtcctgctttgcgacagcaggtgccacgggttgagcggcggttcacacgtttcctacgggttttacgaaaaggatgatttggcgaggtgggctgacgagcttgagaacaggttcggcaaagatctttggatcggggtgcttggcgaatcgctcggagcggcggctgccatcgagctgatgaaacaggatcggagaatcaagttttgtattgccgattcctgcttcagcgatttaacagagctatgccgctttcagctgaaagccgccgtcaaattgtccgtgccgtttctcatcccgttggcaagcgggctgatcaagcggcgccacggctggagccttgctgatatatcgccggtcagcaaccttgagcagtcagacacgccgctgttgatcatccacggcacaaaggatcagcttgttccgccggaaatggccaaaagcctgtatgagaggaaaaagggattcaagaaactgtactggattgaaggcgccggacatgtcggcggatttcgccacaatcctgaggaatacttaaataaaatcaaagactttatcttgaaaatcgagccggccaattag

SEQ ID NO: 61 - DNA, 32 bp, Artificial Sequence
  Oligonucleotide PCR-amplification of pE194
tatatacagctggattcacaaaaaataggcac

SEQ ID NO: 62 - DNA, 33 bp, Artificial Sequence
  Oligonucleotide PCR-amplification of pE194
tatatacagctggattatgtcttttgcgcagtc

SEQ ID NO: 63 - DNA, 30 bp, Artificial Sequence
  Oligonucleotide PCR-amplification of functional mRFP cassette
tatatggatccgtaatcagggtatcgaggc

SEQ ID NO: 64 - DNA, 31 bp, Artificial Sequence
  Oligonucleotide PCR-amplification of functional mRFP cassette
tatatggatccctcattaggcgggctactaa

SEQ ID NO: 65 - DNA, 1027 bp, Artificial Sequence
  Fusion of 5- and 3-prime regions of aprE gene with flanking BsaI sites (homology regions)
ggtctcgacccgaagttcttttttaacatataggtaaaacaatacgaaaaaaggcgccaagtattgaagaattgcagcagccgcggcatttcccttttcgattgaagcaaaaaacgtatattgaacagtaagcattccaaaaatggaaaatactaaaatcgaacaaatatctgtttttttcttccatatctgacacacatgttgaaaaccgtttttcattgaaacatataacaagagaatgactcccgatgccagaagcctgacagagacaagcgagccggcttcaaccgctcccctttcaaatatgtactgtgcagcgcttcccgataatccccacaatgaagcccctgcaagcaccatcaatacgcctttcacatgagctgatttcatatctttcacccgtttctgtatgcgatatattgcatattttaatagatgatcgacaaggccgcaacctccttcggcaaaaaatgatctcataaaataaatgaatagtattttcataaaatgagctcaataacatattctaacaaatagcatatagaaaaagctagtgtttttagcactagctttttcttcattctgatgaaggttgttcaatattttgaatccgttccatgatcgtcggatggccgtatttaaaaatcttgacgagaaacggcgggtttgcctcgctcagcccggcttttgagagctcttgaaacgtcgaaaccgctgcatcgctgttttgcgtcagttcaatcgcatactggtcagcagctttttcctgatgcctcgaaactgcgttcgtaaatggagacgacgcgaaagagatgacccccatcagcatcagaagaagcggaagtgcggctagatcggattttcctgcaatatgaaggcttcttccatagcggccgatgatccgcttgtacagcttgtcgatcacataaaagacagcaagggataaaagcagatacccgccaagtcctatgtaaacatgcttcatcacatagtgccccatttcgtgcgccatgatgctcaggagacc

SEQ ID NO: 66 - DNA, 21 bp, Artificial Sequence
  Oligonucleotide PCR-amplification of aprE genomic region
ccggttgtcattgatccttta

SEQ ID NO: 67 - DNA, 21 bp, Artificial Sequence
  Oligonucleotide PCR-amplification of aprE genomic region
atcctcctgcaaaaaccgtat

SEQ ID NO: 68 - DNA, 1022 bp, Artificial Sequence
  Fusion of 5- and 3-prime regions of amyB gene with flanking BsaI sites (homology regions)
ggtctcgacccacaaggctgtcaaacgaaaaagcgtatcaggagattaacgacacgcaagaaatgatcgaaaaaatcagcggacacctgcctgtacacttgcgtcctccatacggcgggatcaatgattccgtccgctcgctttccaatctgaaggtttcattgtgggatgttgatccggaagattggaagtacaaaaataagcaaaagattgtcaatcatgtcatgagccatgcgggagacggaaaaatcgtcttaatgcacgatatttatgcaacgtccgcagatgctgctgaagagattattaaaaagctgaaagcaaaaggctatcaattggtaactgtatctcagcttgaagaagtgaagaagcagagaggctattgaataaatgagtagaaagcgccatatcggcgcttttcttttggaagaaaatatagggaaaatggtatttgttaaaaattcggaatatttatacaatatcatatgtttcacattgaaaggggaggagaatctagaagagcagagaggacggatttcctgaaggaaatccgtttttttattttgcccgtcttataaatttctttgattacattttataattaattttaacaaagtgtcatcagccctcaggaaggacttgctgacagtttgaatcgcataggtaaggcggggatgaaatggcaacgttatctgatgtagcaaagaaagcaaatgtgtcgaaaatgacggtatcgcgggtgatcaatcatcctgagactgtgacggatgaattgaaaaagcttgttcattccgcaatgaaggagctcaattatataccgaactatgcagcaagagcgctcgttcaaaacagaacacaggtcgtcaagctgctcatactggaagaaatggatacaacagaaccttattatatgaatctgttaacgggaatcagccgcgagctggaccgtcatcattatgctttgcagcttgtcacaaggaaatctctcaatatcggccagtgcgacggcattatctcaggagacc

SEQ ID NO: 69 - DNA, 22 bp, Artificial Sequence
  Oligonucleotide PCR-amplification of amyB genomic region
agaagcatgaagggcatgcgac

SEQ ID NO: 70 - DNA, 21 bp, Artificial Sequence
  Oligonucleotide PCR-amplification of amyB genomic region
caacaacaggctgtctgacgg

SEQ ID NO: 71 - DNA, 1022 bp, Artificial Sequence
  Fusion of 5- and 3-prime regions of sigF gene with flanking BsaI sites (homology regions)
ggtctcgacccaaaatcattcgccttgagcaatcagagcagcgtgcacttgaaacgttgggggtggcgtcatgaaaaatgaaatgaacattcagtttacagcgctcagccaaaatgaatcgtttgcacgggtgacagtcgctgcttttatcgctcagcttgacccgacgatggatgaactgaccgaaattaaaacggtcgtatccgaagcggtcacaaacgcgatcattcacggttatgaaaactcagggcagggaaacgtatatatttccgtcactctcgaggaccatattgtctatttaacgatccgcgacgaaggagtcggcatccctgatcttgaagaagcgcgccagcccctgttcacgacaaagcctgaactcgagcggtcgggaatgggctttacgatcatggaaaatttcatggatgatatttcgatcgactcctcacctgagatgggaaccacaatacacttaacaaagcacttatcaaaaagcaaagcgctttgcaattaatgaggctgctcatgttgcaggcagcctcggatgtccgatgaaaaaccggacgctcttgggagcgttccggttttttttgtgtggtaatttatggtcttttgcgcctttctgaatgataaatgggatgtacttcatactacaactataaccatcatataggaagtgacccagatggaacgtcaagtttttatcagactccgccaccggcttgaggcagatccggatgaattgattttgctcggccatatcgcacaggtagcgggtgaccgcggatacaaagaaaagcttgaacggctgcctatttatcaggtcagcaaagcggatcaaagcatggtggtgctggacgtgatgaaggtgattgaagctgtacataaatcgtttcctgaccttgatgtccaaaccgtcggcggttctgaaaccatcgtggagatccaatatccgaaaaggggtctgtcgcccgtgcttttcatcgccgtctggctgctcttgttcgtcggtgcctcaggagacc

SEQ ID NO: 72 - DNA, 19 bp, Artificial Sequence
  Oligonucleotide PCR-amplification of sigF genomic region
agcagctcggcggagaaat

SEQ ID NO: 73 - DNA, 21 bp, Artificial Sequence
  Oligonucleotide PCR-amplification of sigF genomic region
accttgcccgtcaccatttcg

SEQ ID NO: 74 - DNA, 1363 bp, Artificial Sequence
  Fusion of 5- and 3-prime regions of pga genes with flanking BsaI sites (homology regions)
ggtctcgacccgaacactgaaattttagaccgggctgggatatcgagacatatataggggcgtttaatggcgaatacagtaacaatgagaatagtaagaaaaattaaaaatgttaaagtttgatgaattatcattgaaaaaaattaatggctttttaaatcctaggattttaacctaaaatctgaagaaataaggtggatcgaacgactcacaaaatatttggatttgtcaatgaatcccgctttatgctaaaagagattttcattttttgatagatggtctgattgtcataggacggatttgttttgaagagggaacattggtgactttttaacctgttcgaaaagagcgaaaatactaaaagaaaagagacatcccggctgacagcccatttaaaggggattgcggccgggggaaaaaagagatcctgaatccatccttcaacctttcatctgaaatagggagaaaagtacaaaaatcataatgtcgaattttgaaagcgcatacttaaaacgctgacaaaaatctgataggaattaagaactttcgatttccaaaaatatcaataaaaagataggcattaatgactcgggcgaggtgatctttgtcacggaaaatttcgtcgtcttctgttacataatgccgattgtgatttcatagtgaaccctgatcccggttataaaagacctgtgaaaagcggccggtttgaaagggaaacacgacaattttcttaaccggtcagtgtataaagttttatagaaaatcaggaggatatatacatggttttggggttcatgtttattgtattcttttgaagggaataaaaactgacaaatttcgactgaagcaaaatttgaaaatgcatcaccttaccaattcgggatgggaaccgcacctcatgttcatgacctctttagaatatttcccttcatctttttaatccgcgcttaggtgaaaaagctgatcatgctgtgctgagcgtttcttctcgctatgacgctgctgtacatgcaaaaaaagtcctttaaatatcccagttgaatgacgatgaaagaggaaagaagaggaggaacagatcaattgataaaaaaagcggcaaacaaaaagttggttttgttttgtggaattgcggtgctttggatgtctttatttttaacgaatcataatgatgtacgcgccgatacgatcggcgagaaaatagcggaaactgccagacagcttgagggtgcgaaatacagctacggcggagagaagccgaaaacggggtttgactcgtcaggctttgtgcaatatgtgtttcaatcgctcgatattacgcttccgagaacggtaaaggaacaatcgactcttggctcaggagacc

SEQ ID NO: 75 - DNA, 20 bp, Artificial Sequence
  Oligonucleotide PCR-amplification of pga genomic region
aaagccttctcctctctatt

SEQ ID NO: 76 - DNA, 20 bp, Artificial Sequence
  Oligonucleotide PCR-amplification of pga genomic region
ttcttgaaaaagacaaggtc

SEQ ID NO: 77 - DNA, 37 bp, Artificial Sequence
  Oligonucleotide PCR-amplification of yitV 5-prime homologous region
agagaaggtctcaacccttcaaaccggagaatgtgcc

SEQ ID NO: 78 - DNA, 33 bp, Artificial Sequence
  Oligonucleotide PCR-amplification of yitV 5-prime homologous region
ttctctggtctcggtttgacgcgcacttgtaag

SEQ ID NO: 79 - DNA, 42 bp, Artificial Sequence
  Oligonucleotide PCR-amplification of yitV 3-prime homologous region
agagaaggtctcaaaacccatcatcataagtttaacatgtag

SEQ ID NO: 80 - DNA, 38 bp, Artificial Sequence
  Oligonucleotide PCR-amplification of yitV 3-prime homologous region
cctctctggtctcatgagctgcccatcgtaaagacatc

SEQ ID NO: 81 - DNA, 35 bp, Artificial Sequence
  Oligonucleotide PCR-amplification of ypmR 5-prime homologous region
agagaaggtctcaaccccgtttttaagcggcatct

SEQ ID NO: 82 - DNA, 32 bp, Artificial Sequence
  Oligonucleotide PCR-amplification of ypmR 5-prime homologous region
ttctctggtctcgaaacgaagacctcccagca

SEQ ID NO: 83 - DNA, 41 bp, Artificial Sequence
  Oligonucleotide PCR-amplification of ypmR 3-prime homologous region
agagaaggtctcagtttgtgtaccatgttttgattcttttg

SEQ ID NO: 84 - DNA, 40 bp, Artificial Sequence
  Oligonucleotide PCR-amplification of ypmR 3-prime homologous region
cctctctggtctcatgagggtggctgactttatatttacg